(19) United States
(12) Patent Application Publication
(10) Pub. No.: US 2026/0078436 A1
(43) Pub. Date: Mar. 19, 2026

(54) COMPOSITIONS AND METHODS FOR STRAND-DISPLACEMENT AMPLIFICATION OF TARGET NUCLEIC ACIDS

(71) Applicant: 10x Genomics, Inc., Pleasanton, CA (US)

(72) Inventors: Peter Smibert, Danville, CA (US); Marco Serra, Oakland, CA (US)

(21) Appl. No.: 19/327,731

(22) Filed: Sep. 12, 2025

Related U.S. Application Data
(60) Provisional application No. 63/694,072, filed on Sep. 12, 2024.
(60) Provisional application No. 63/738,508, filed on Dec. 23, 2024.

(51) Int. Cl. C12Q 1/6855

(52) U.S. Cl. CPC C12Q 1/6855

(57) ABSTRACT

Methods for strand-displacement amplification of a target nucleic acid have been developed. Compositions for use according to the methods are also provided. The methods typically include steps of removing, hybridizing and extending a selectively removable or digestible primer with a strand-displacing DNA polymerase once or more than once to provide a multiplicity of copies of the target nucleic acid. Methods for strand-displacement amplification of single or double-stranded target DNA molecules are provided. In some forms, when the methods include amplification of a double-stranded nucleic acid, the methods employ one or more adapter handles that perform the function of providing a removable region on a single strand of the ds target. Compositions and kits of oligonucleotide primers and double-stranded nucleic acid adapters including selectively removable regions are also provided for use in the described methods.

### CROSS-REFERENCE TO RELATED APPLICATIONS

This application claims the benefit of and priority to U.S. Provisional Application No. 63/694,072, filed Sep. 12, 2024, and U.S. Provisional Application No. 63/738,508, filed Dec. 23, 2024, which are hereby incorporated herein by reference in their entireties.

### FIELD OF THE DISCLOSURE

The present disclosure is generally in the field of molecular profiling of analytes present in a biological sample, specifically enhanced compositions, methods, and systems for amplification of target nucleic acids.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling and cross-talk with other cells in the tissue.

Spatial heterogeneity has been previously studied using techniques that only provide data for a small handful of analytes in the context of an intact tissue or a portion of a tissue, or provide substantial analyte data for dissociated tissue (i.e., single cells), but fail to provide information regarding the position of the single cell in a parent biological sample (e.g., tissue sample).

Current methods for spatial profiling of analytes include preparation of a library of nucleic acid probes including a sequence corresponding to an RNA from within a biological sample, together with a barcode that imparts spatial information regarding the location of the RNA within the sample. Typically, the library is generated from a plurality of nucleic acid probes conjugated to a substrate. While library preparation and applications may be enhanced by amplification of target nucleic acids, the low-fidelity of classical polymerase-chain rection (PCR) methods may introduce undesirable variant nucleic acid molecules that are amplified together with the original target sequence, resulting in a mixture of different sequences that can reduce accuracy and efficiency of spatial profiling. In addition, the necessity to vary the temperature during “thermocycling” that is required throughout PCR can lead to undesirable nucleic acid interactions, as well as prolonged sample preparation times.

Therefore, there is a need for enhanced methods for amplification of target nucleic acids for spatial analysis of analytes (e.g., nucleic acids and/or proteins) in biological samples.

There is also a need for methods to amplify target nucleic acids that reduce the number and/or extent of variations in temperature and/or other reaction conditions.

There is also a need for methods to accurately and reliably amplify target nucleic acids conjugated to a substrate.

### SUMMARY

Compositions and methods for strand-displacement amplification of a target nucleic acid have been developed.

In some forms, methods for amplification of a target DNA molecule include the steps of (a) hybridizing a first primer to a first primer binding site in a target DNA molecule, the first primer including: (i) a first region, including a DNA sequence that is substantially complementary to the first primer binding site of the target DNA molecule; and (ii) a second hybrid region including one or more of RNA nucleotides, DNA nucleotides including a unique target sequence, or inosine nucleotides, whereby the target DNA molecule includes single stranded (ss) DNA including the first primer binding site; (b) extending the first primer from its 3′ end using the target DNA molecule as a template and extending 3′ end of the target DNA molecule using the first primer as a template, to form an extended hybrid double-stranded (ds) DNA molecule; (c) removing the second hybrid region from the extended hybrid dsDNA molecule to form a truncated dsDNA molecule including a region of ssDNA; (d) hybridizing a second primer to the region of ssDNA of the truncated dsDNA molecule to form a hybridized second molecule including a hybridized second primer, whereby the second primer includes the second hybrid region including one or more of RNA nucleotides, DNA nucleotides including a unique target sequence, or inosine nucleotides; and (c) extending the hybridized second primer of the hybridized second molecule using a strand-displacing DNA polymerase to provide an extended hybrid double-stranded (ds) DNA molecule and a displaced single-stranded (ss) DNA molecule. In some forms, the first region is located at the 3′ end of the first primer, and/or the first primer binding site is located at the 3′ end of the target DNA molecule. In some forms, the second hybrid region of the first primer forms a single stranded 5′ overhang when the first primer hybridizes to the target DNA molecule. In some forms, the first primer includes in 5′ to 3′ orientation: the second hybrid region and the first region.

In certain forms, the methods amplify a target DNA molecule derived from a template-switching workflow. For example, in some forms, the methods include the steps of: (a) forming a target ssDNA molecule from a template-switching workflow, including attaching a template switching oligonucleotide to a captured probe to form the target nucleic acid, whereby the template switching oligonucleotide further includes a second primer binding sequence, and whereby the generated target ssDNA molecule further includes the second primer binding sequence or a complement thereof; (b) hybridizing a second primer to the second primer binding sequence of the target ssDNA molecule to form a hybridized second molecule including a hybridized second primer, whereby the second primer includes a second hybrid region including one or more of RNA nucleotides, DNA nucleotides including a unique target sequence, or inosine nucleotides; (c) extending the hybridized second primer of the hybridized second molecule using a strand-displacing DNA polymerase to provide an extended hybrid double-stranded (ds) DNA molecule; (d) removing the second primer from the extended hybrid double-stranded (ds) DNA molecule to form a truncated dsDNA molecule; (c) hybridizing a second primer to the second primer binding sequence of the truncated dsDNA molecule to form a hybridized second molecule including a hybridized second primer; and (f) extending the hybridized second primer of the hybridized second molecule using a strand-displacing DNA polymerase to provide an extended hybrid double-stranded (ds) DNA molecule and a displaced single-stranded (ss) DNA molecule. In some forms, the methods further include, prior to step (a), forming the target DNA molecule that includes ssDNA from a blocked probe workflow. In some forms, a 5′ end of the target DNA molecule is conjugated to a substrate.

In certain forms, the methods amplify a single strand of a double-stranded target DNA molecule. For example, in some forms the methods include the steps of: (a) forming a combined first molecule from a target double stranded (ds) DNA molecule including a first DNA strand and a second DNA strand, the forming including: (i) linking to a 3′ end of the second DNA strand of the target dsDNA molecule a nucleic acid adapter, the nucleic acid adapter including: (I) a first adapter strand, including: (i) a first region, including DNA nucleotides; and (ii) a second hybrid region, including RNA nucleotides, or DNA nucleotides including a unique target sequence, or inosine nucleotides; and (II) a second adapter strand including a DNA sequence complementary to all or part of the first strand; whereby the first adapter strand is hybridized to the second adapter strand (in some forms, the second adapter strand is linked to the 3′ end of the second DNA strand via a 5′ phosphodiester linkage); (b) removing the second hybrid region from the combined first molecule to form a truncated DNA molecule including a region of ssDNA; (c) hybridizing a second primer to the region of ssDNA of the truncated DNA molecule to form a hybridized second molecule including a hybridized second primer, whereby the second primer includes the second hybrid region including RNA nucleotides, DNA nucleotides including a unique target sequence, or inosine nucleotides; and (d) extending the hybridized second primer with a strand-displacing DNA polymerase to provide an extended hybrid dsDNA molecule and a displaced DNA molecule. In other forms the methods to amplify a single strand of a double-stranded target DNA molecule include the steps of: (a) forming a combined first molecule having a 3′overhang from a target double stranded (ds) DNA molecule having a first DNA strand and a second DNA strand, the forming including: linking to a 5′ end of the first DNA strand of the target dsDNA molecule a 3′ end of a first adapter strand of a partially ds nucleic acid adapter, the partially ds nucleic acid adapter including: (i) a first adapter strand, including a first region of DNA nucleotides; and (ii) a second adapter strand, including a DNA sequence complementary to the first region of DNA nucleotides of the first strand and a second region of DNA nucleotides, whereby the second region includes at least 5 nucleotides; whereby the second adapter strand is hybridized to the first region of DNA nucleotides of the first adapter strand to form the partially ds nucleic acid adapter including a 3′ overhang, and whereby the 3′ overhang includes the second region of DNA nucleotides of the first adapter strand; (optionally whereby the second adapter strand is linked to the 3′ end of the second DNA strand via a 5′ phosphodiester linkage); (b) hybridizing a second primer to 3′ overhang of the combined first molecule to form a hybridized second molecule including a hybridized second primer, whereby the second primer includes a sequence complementary to the second region of DNA nucleotides of the first adapter strand, and whereby the second primer includes RNA nucleotides, DNA nucleotides including a unique target sequence, or inosine nucleotides; (c) extending the hybridized second primer with a strand-displacing DNA polymerase to provide an extended hybrid dsDNA molecule and a displaced ssDNA molecule. In some forms, the methods further include (d) repeating step (b) and/or (c). In some forms, forming in step (a) includes blunt end ligating the second adapter strand to the second DNA strand of the target dsDNA molecule.

In other forms the methods to amplify a single strand of a double-stranded target DNA molecule include the steps of: (a) forming a combined first molecule from a target double-stranded (ds) DNA molecule and a nucleic acid adapter, whereby the target dsDNA molecule includes a first DNA strand and a second DNA strand, the forming including: (i) linking to a 3′ end of the second DNA strand of the target dsDNA molecule a 5′ end of a second strand of the nucleic acid adapter, to form a linked second adapter strand, and (ii) hybridizing a first strand of the nucleic acid adapter to the linked second adapter strand to form the combined first molecule, whereby the first strand of the nucleic acid adapter includes (I) a first region including DNA nucleotides; and (II) a second hybrid region, including RNA nucleotides, DNA nucleotides including a unique target nucleotide sequence, or inosine nucleotides, or a methylated GATC nucleotide sequence, whereby the first strand of the nucleic acid adapter includes a nucleotide sequence complementary to all or part of the second strand of the nucleic acid adapter (optionally whereby a 5′ end of the second strand of the nucleic acid adapter is linked to a 3′ end of the second DNA strand of the dsDNA molecule via a phosphodiester linkage); (b) removing the second hybrid region from the combined first molecule to form a truncated DNA molecule including a region of ssDNA; (c) hybridizing a second primer to the region of ssDNA of the truncated DNA molecule to form a hybridized second molecule including a hybridized second primer, whereby the second primer includes the second hybrid region including RNA nucleotides, DNA nucleotides including a unique target sequence, or inosine nucleotides; (d) extending the hybridized second primer with a strand-displacing DNA polymerase to provide an extended hybrid dsDNA molecule and a displaced DNA molecule. In some forms, the linking to a 3′ end of the first DNA strand of the dsDNA molecule a nucleic acid adapter includes ligation, optionally whereby the ligation includes enzymatic ligation. In some forms, the second adapter strand includes a 5′ phosphate moiety, and whereby the second adapter strand is blunt end ligated to the 3′ end of the second DNA strand of the target dsDNA molecule. In some forms, the adapter is partially double stranded, and/or whereby 5′ end of the second strand of the double stranded nucleic acid adapter does not include a 5′ phosphate. In some forms, a 5′ end of the first strand of the target dsDNA molecule is conjugated to a substrate. In some forms, whereby the displaced ssDNA molecule includes a sequence of, or complementary to, that of the target DNA molecule. In some forms, the strand-displacing DNA polymerase is selected from the group including Bst2.0, Bst3.0, Bsu, and Phil29. In some forms, removing the second hybrid region includes contacting the second hybrid region with an enzyme that selectively degrades the second hybrid region. In some forms, the second hybrid region includes RNA nucleotides, and removing the second hybrid region includes contacting the second hybrid region with an RNase enzyme. An exemplary RNase enzyme includes RNase H. In some forms, the second hybrid region includes one or more inosine nucleotides, and removing the second hybrid region includes contacting the second hybrid region with an endonuclease enzyme that cleaves the one or more inosine nucleotides. An exemplary endonuclease includes Endonuclease V. In some forms, the second hybrid region includes one or more unique target sequences, whereby the one or more unique target sequences are not present within the target DNA molecule, and removing the second hybrid region includes contacting the second hybrid region with a nickase enzyme that selectively cleaves or removes the one or more unique target sequences. In some forms, the second hybrid region includes one or more methylated GATC sequences, and removing the second hybrid region includes contacting the second region with a methylation-specific nuclease. An exemplary methylation-specific nuclease includes DpnI.

In some forms, forming in step (a) includes contacting the target DNA molecule with a reaction mixture including the first primer, or partially double stranded adapter, or double stranded adapter and the strand displacing DNA polymerase. In some forms, the reaction mixture further includes one or more of: (i) an enzyme specific for the second hybrid region; (ii) and the second primer. In some forms, the reaction mixture further includes (i) the second primer; (ii) the enzyme specific for the second hybrid region; and (iii) the strand displacing DNA polymerase. In some forms, the methods repeat each of the steps of removing, hybridizing and extending the second primer with a strand-displacing DNA polymerase once or more than once to provide a multiplicity of copies of the target DNA molecule. In some forms, each of the steps of removing, hybridizing and extending the second primer with a strand-displacing DNA polymerase is carried out at a constant temperature. In some forms, the target DNA molecule includes a spatial barcode and/or a DNA sequence of at least a portion of a target nucleic acid analyte from a sample, or a complement thereof. In some forms, the target nucleic acid analyte includes a nucleic acid. An exemplary analyte is selected from genomic DNA, RNA, synthetic DNA, or synthetic RNA. In some forms, the target nucleic acid analyte is derived from a biological sample, such as a biological sample derived from a human subject. In some forms, the target nucleic acid analyte includes an RNA selected from small interfering RNA (siRNA), microRNA (miRNA), β-element-induced wimpy testis (PIWI)-interacting RNA (piRNA), small nucleolar RNA (snoRNA), small nuclear RNA (snRNA), messenger RNA (mRNA), ribosomal RNA (rRNA), long non-coding RNAs (lncRNA), and transfer RNA (tRNA). In an exemplary form, the RNA includes mRNA.

In some forms, the target DNA molecule is directly or indirectly conjugated to a substrate or matrix. An exemplary substrate or matrix includes a solid support or a gel. In certain forms, the substrate includes an array including a multiplicity of capture probes. In some forms, the target DNA molecule is conjugated to the substrate or matrix via a first cleavable linker. Exemplary first cleavable linkers are selected from the group including a photocleavable linker, UV-cleavable linker, or an enzyme-cleavable linker. In some forms, the first cleavable linker is an enzyme-cleavable linker. In some forms, the target DNA molecule includes a spatial barcode and/or at least a portion of a target nucleic acid analyte or a complement thereof, and a substrate is conjugated to 5′end of the target DNA molecule; and/or the first primer binding site is located at the 3′ end of the target DNA molecule. In some forms, the target DNA molecule includes a spatial barcode and/or at least a portion of a target nucleic acid analyte or a complement thereof, and (i) a substrate is conjugated to the 5′end of a first strand of the target DNA molecule; and (ii) the second strand of the double stranded nucleic acid adapter is linked to the 3′ end of the first strand of the target dsDNA molecule. In some forms, the target DNA molecule includes one or more of (i) a unique molecular identifier (UMI); (ii) a spatial barcode; (iii) a capture domain; and (iv) a sequence of at least a portion of a target analyte, or a complement thereof. In some forms, the methods further including determining the nucleic acid sequence of the displaced DNA molecule. In some forms, determining the nucleic acid sequence includes determining the sequence of one or more of (i.) a spatial barcode or a complement thereof; (ii.) all or a portion of a target analyte or a complement thereof; and/or (iii.) a molecular identifier (UMI). In some forms, the methods further include using the determined sequences of (i.), (ii.) and optionally (iii.) to identify the location and/or abundance of the target nucleic acid analyte in a biological sample. In some forms, the methods further include, prior to step (a), contacting a biological sample with one or more permeabilizing reagents, whereby the biological sample includes a nucleic acid having a nucleotide sequence within the target nucleic acid analyte, or a complement thereof. In some forms, the permeabilizing reagent is selected from an organic solvent, a detergent, and an enzyme, or a combination thereof.

In some forms, the methods further include, immediately prior to step (a), contacting an array with an exonuclease enzyme to remove un-extended oligos with free 3′ ends from array. In some forms, the methods further include, immediately prior to step (a), modifying the free 3′ ends on the array by the addition of inverted dT or inverted dideoxy dT. In some forms, the methods further include, immediately prior to step (a), contacting the array with a plurality of poly(A) oligonucleotides, whereby the plurality of poly(A) oligonucleotides bind to free poly(T) nucleotides in the array.

Methods for amplification of a target DNA molecule are also provided. The methods include the steps of:
(a) extending a capture probe using a target nucleic acid analyte hybridized thereto as a template, thereby generating an extended capture probe including all or a portion of a complement of the target nucleic acid analyte, optionally whereby the target nucleic acid analyte is RNA;
(b) performing a template switching reaction using (i) the extended capture probe and (ii) a template switching oligonucleotide including a first primer binding sequence at its 5′ end, thereby generating a target DNA molecule including the extended capture probe and a complement of the first primer binding sequence;
(c) hybridizing a second primer to the complement of the first primer binding sequence of the target DNA molecule, whereby the second primer includes a second hybrid region corresponding to the first primer binding sequence, and whereby the second hybrid region includes RNA nucleotides, or DNA nucleotides including a unique target sequence, or inosine nucleotides, or a methylated GATC nucleotide sequence;
(d) extending the second primer using the target DNA molecule as a template, to form a double stranded (ds) hybrid DNA molecule;
(c) removing the second hybrid region of the second primer from the ds hybrid DNA molecule to form a truncated DNA molecule including a ssDNA region;
(f) hybridizing a second primer including the second hybrid region to the ssDNA region of the truncated DNA molecule to form a hybridized second primer; and
(g) extending the hybridized second primer with a strand-displacing DNA polymerase to provide an extended nucleic acid molecule and a displaced DNA molecule. In some forms, the displaced DNA molecule includes all or a portion of the sequence of the target DNA molecule. In some forms, the template switching oligonucleotide further includes a second primer binding sequence, and whereby the generated target DNA molecule further includes a complement of the second primer binding sequence. In some forms, the step of extending the capture probe includes reverse transcription, optionally whereby one or more non-templated nucleotides are incorporated at the 3′ end of the extended capture probe during the reverse transcription and optionally whereby in step (b) the template switching oligonucleotide hybridizes to the one or more non-templated nucleotides.

Compositions of oligonucleotide primers and fully or partially double stranded (ds) nucleic acid adapters for use according to the described methods for strand displacement amplification of a target nucleic acid are also provided. In some forms, the size of the first primer or single stranded adapter or second strand of the double stranded adapter is between about 8 and about 100 nucleotides, inclusive. In other forms, the second hybrid region includes between about four and about 30 RNA nucleotides, inclusive. In other forms, the first region includes between about four and about 30 DNA nucleotides, inclusive. In some forms, the target DNA molecule includes a region complementary to a poly(A) nucleotide sequence, or a complement thereof.

Compositions of target nucleic acids, and amplification products thereof prepared according to the described methods for strand displacement amplification of a target nucleic acid are also provided. In some forms, the target DNA molecule includes a nucleotide sequence of a coding region of an mRNA, or a complement thereof. In some forms, the target DNA molecule includes one or more functional domains. Exemplary functional domains include an amplification domain and/or a primer-binding site. In some forms, the target DNA molecule is formed from or includes one of a plurality of arrayed capture probes. In some forms, the target DNA molecule includes one or more priming sites for a sequencing primer. In some forms, the target DNA molecule is derived from a tissue sample, for example, derived from a mammalian subject, such as a human subject.

Compositions of oligonucleotide primer sets for use according to the described methods for strand displacement amplification of a target nucleic acid are also provided. Typically, the oligonucleotide primer sets include: (a) a first oligonucleotide primer, including (i) a first region including DNA bases complementary to a first primer binding site of a target nucleic acid; and (ii) a second hybrid region, whereby the second region includes one or more of RNA nucleotides, DNA nucleotides including a unique target sequence, inosine nucleotides, or a methylated GATC sequence; and (b) a second oligonucleotide primer, including the second hybrid region of the first oligonucleotide primer. In some forms, the second oligonucleotide primer is formed of the second hybrid region of the first oligonucleotide primer. In some forms, the size of the first oligonucleotide primer is between about 8 and about 100 nucleotides, inclusive. In other forms, the first oligonucleotide primer includes a first region including between about four and about 30 nucleotides, inclusive. In other forms, the first oligonucleotide primer includes a second region including between about four and about 30 nucleotides, inclusive. In some forms, the first region of the first oligonucleotide primer includes one or more locked nucleic acids (LNA). In some forms, the second region of the first oligonucleotide primer includes RNA nucleotides. In some forms, the second region of the first oligonucleotide primer includes inosine nucleotides. In some forms, the second region of the first oligonucleotide primer includes a unique target sequence, and whereby the target sequence includes a nickase recognition sequence. In some forms, the second region of the first oligonucleotide primer includes a methylated GATC nucleotide sequence.

Kits of parts for use according to the described methods for strand displacement amplification of a target nucleic acid are also provided. Typically, the kits include: (a) a first oligonucleotide primer, including (i) a first region including DNA bases complementary to a first primer binding site of a target nucleic acid; and (ii) a second hybrid region, whereby the second region includes one or more of RNA nucleotides, DNA nucleotides including a unique target sequence, inosine nucleotides, or a methylated GATC sequence; and (b) instructions for performing any one of the described methods for strand displacement amplification of a target nucleic acid. In some forms, the second region of the first primer includes RNA nucleotides. In some forms, the kit includes a second primer including the second region of the first primer. In some forms, the kit further includes an RNA-specific nuclease enzyme. In some forms, the kit further includes an RNA-specific nuclease enzyme, such as RNaseH. In some forms, the second region of the first primer includes inosine nucleotides. In some forms, the kit further includes a second primer including the second region of the first primer. In some forms, the kit further includes an endonuclease enzyme that degrades the inosine nucleotides, such as Endonuclease V. In some forms, the second region of the first primer includes one or more unique target sequences, the kit further includes a second primer including the second region of the first primer, and the kit further includes a nickase enzyme that selectively cleaves or removes the unique target sequences. In some forms, the second region of the first primer includes one or more methylated GATC nucleotide sequences and the kit further includes a second primer including the second region of the first primer. In some forms, the kit further includes a methylation-specific nuclease enzyme, such as DpnI. In some forms, the kit further includes a single stranded adapter or a double stranded adapter. In some forms, the adapter includes bases complementary to the second region of the first primer. In some forms, the first adapter includes a 5′phosphate.

In some forms, the kit further includes a ligase enzyme, such as a T4 ligase. In some forms, the kit further includes a strand displacing DNA polymerase, such as a strand-displacing polymerase is selected from Bst2.0, Bst3.0, Bsu and Phil29.

In some embodiments, the methods provided herein may involve hybridizing a probe to a molecule of interest (e.g., target protein, target nucleic acid molecule) and processing the probe-molecule complex. Such processing can include barcoding the probe, the probe-molecule complex, or the molecule, and/or performing a nucleic acid reaction. The probe may comprise a nucleic acid molecule, and further processing can include extension, denaturation, and amplification processes to provide nucleic acid molecules comprising a sequence the same or substantially the same as or complementary to that of a target region of a nucleic acid molecule of interest (e.g., target nucleic acid molecule).

Further disclosed herein are methods comprising hybridizing a first probe and a second probe to first and second target regions of the nucleic acid molecule, linking the first and second probes to provide a probe-linked nucleic acid molecule, and barcoding the probe-linked nucleic acid molecule. A method may comprise hybridizing a first probe to a first target region of a nucleic acid molecule, barcoding the probe, and hybridizing a second probe to a second target region of the nucleic acid molecule to generate a barcoded, probe-linked nucleic acid molecule. In some aspects, the method may comprise hybridizing a probe to a nucleic acid molecule attached to a feature-binding moiety to provide a probe-binding moiety complex and barcoding the probe. One or more processes of the methods provided herein may be performed within a partition such as a droplet or well. The methods of the present disclosure be useful, for example, in controlled analysis and processing of analytes such as biological particles, nucleic acids, and proteins. One or more of the methods described herein may allow for genomic, transcriptomic, or exomic profiling with higher sensitivity. The methods of the present disclosure may be useful in detecting variants and characterizing nucleic acid molecules, e.g., for assessment of single nucleotide polymorphisms (SNPs), alternative splice junctions, insertions, deletions, V (D) J rearrangements, etc. The methods of the present disclosure may be useful for multiplexed analysis of nucleic acids and proteins while minimizing reagent usage, e.g., by decreasing the number of unoccupied partitions for analysis.

In some aspects, molecules of interest comprise nucleic acid molecules including deoxyribonucleic acid (DNA) or ribonucleic acid (RNA). In some embodiments, such nucleic acid molecules of interest include target nucleic acid molecules that are attached to a substrate such as a particle or bead. In some embodiments, nucleic acid molecules of interest include nucleic acids, such as DNA, comprising probes, labels, barcodes or other molecular tags. Such molecular tags and barcodes can allow for identification and/or quantification of various DNA molecules. In some embodiments, a barcoded nucleic acid molecule, such as a barcoded DNA molecule attached to a particle, may be amplified prior to further downstream analysis, such as analysis by sequencing or other molecular approaches.

In some aspects, the present disclosure is related to methods for amplification of a target DNA molecule, comprising: (a) hybridizing a first primer to a first primer binding site in a target double stranded (ds) DNA molecule, the first primer comprising: (i) a first region, comprising a DNA sequence that is substantially complementary to the first primer binding site of the target DNA molecule, wherein the first region of the first primer displaces dsDNA at the first primer binding site; and (ii) a second hybrid region comprising one or more of RNA nucleotides, DNA nucleotides comprising a unique target sequence, or inosine nucleotides; (b) extending 3′ end of the target DNA molecule using the first primer as a template, to form an extended hybrid double-stranded (ds) DNA molecule; (c) removing the second hybrid region from the extended hybrid dsDNA molecule to form a truncated dsDNA molecule comprising a region of ssDNA; (d) hybridizing a second primer to the region of ssDNA of the truncated dsDNA molecule to form a hybridized second molecule comprising a hybridized second primer, wherein the second primer comprises the second hybrid region comprising one or more of RNA nucleotides, DNA nucleotides comprising a unique target sequence, or inosine nucleotides; and (e) extending the hybridized second primer of the hybridized second molecule using a strand-displacing DNA polymerase to provide an extended hybrid double-stranded (ds) DNA molecule and a displaced single-stranded (ss) DNA molecule.

In some embodiments, the first primer includes a first region comprising one or more modified nucleic acids, wherein the modified nucleic acids include, but are not limited to, locked nucleic acids (LNAs), super G (8-aza-7-deazaguanosine) nucleic acids, or the like.

In some embodiments, the first region is located at the 3′ end of the first primer.

In some embodiments, the second hybrid region of the first primer forms a single stranded 5′ overhang when the first primer hybridizes to the target DNA molecule.

In some embodiments, the first primer comprises in 5′ to 3′ orientation: the second hybrid region and the first region.

In some aspects, the present disclosure is related to methods for amplification of a target DNA molecule, comprising: (a) forming a combined first molecule from a target partially double stranded (ds) DNA molecule comprising a first DNA strand and a second DNA strand, the forming comprising: (i) linking to a 5′ end of the second DNA strand of the target dsDNA molecule a partially double stranded nucleic acid adapter, the nucleic acid adapter comprising: (I) a first adapter strand, comprising: (i) a first region, comprising DNA nucleotides; and (ii) a second hybrid 5′ overhang region, comprising RNA nucleotides, or DNA nucleotides comprising a unique target sequence, or inosine nucleotides; and (II) a second adapter strand comprising a 3′ overhang DNA splint sequence, wherein the splint sequence is complementary to all or part of the first adapter strand and 5′ end of the second DNA strand of the target partially dsDNA molecule; wherein the first adapter strand is hybridized to the second adapter strand; and (i) extending 3′end of the second DNA strand of the target dsDNA molecule using the first adapter strand as a template, wherein extending 3′end of the second DNA strand of the target dsDNA molecule displaces the DNA splint sequence; (b) removing the second hybrid region from the combined first molecule to form a truncated DNA molecule comprising a region of ssDNA; (c) hybridizing a second primer to the region of ssDNA of the truncated DNA molecule to form a hybridized second molecule comprising a hybridized second primer, wherein the second primer comprises the second hybrid region comprising RNA nucleotides, DNA nucleotides comprising a unique target sequence, or inosine nucleotides; (d) extending the hybridized second primer with a strand-displacing DNA polymerase to provide an extended hybrid dsDNA molecule and a displaced DNA molecule.

In some embodiments, the target DNA molecule is directly or indirectly conjugated to a particle. In some embodiments, the particle is a bead. In some embodiments, the particle is a gel bead. In some embodiments, the particle comprises a barcode sequence.

In some embodiments, the displaced DNA molecule comprises a sequence of, or complementary to that of the target DNA molecule.

In some embodiments, the strand-displacing DNA polymerase is selected from the group consisting of Bst2.0, Bst3.0, Bsu and Phil29.

In some embodiments, removing the second hybrid region comprises contacting the second hybrid region with an enzyme that selectively degrades the second hybrid region. In some embodiments, the second hybrid region comprises RNA nucleotides, and removing the second hybrid region comprises contacting the second hybrid region with an RNase enzyme. In some embodiments, the RNase enzyme comprises RNase H.

In some embodiments, the second hybrid region comprises one or more inosine nucleotides, and removing the second hybrid region comprises contacting the second hybrid region with an endonuclease enzyme that cleaves the one or more inosine nucleotides. In some embodiments, the endonuclease comprises Endonuclease V.

In some embodiments, the second hybrid region comprises one or more unique target sequences, wherein the one or more unique target sequences are not present within the target DNA molecule, and removing the second hybrid region comprises contacting the second hybrid region with a nickase enzyme that selectively cleaves or removes the one or more unique target sequences.

In some embodiments, the second hybrid region comprises one or more methylated GATC sequences, and removing the second hybrid region comprises contacting the second region with a methylation-specific nuclease. In some embodiments, the methylation-specific nuclease comprises DpnI.

In some embodiments, the forming in step (a) comprises contacting the target DNA molecule with a reaction mixture comprising the first primer, or partially double stranded adapter, and the strand displacing DNA polymerase. In some embodiments, the reaction mixture further comprises one or more of:
(i) an enzyme specific for the second hybrid region;
(ii) and the second primer.

In some embodiments, the methods further comprise repeating each of the steps of removing, hybridizing and extending the second primer with a strand-displacing DNA polymerase once or more than once to provide a multiplicity of copies of the target DNA molecule. In some embodiments, each of the steps of removing, hybridizing and extending the second primer with a strand-displacing DNA polymerase is carried out at a constant temperature.

In some embodiments, the target DNA molecule comprises a barcode and/or a DNA sequence of at least a portion of a target nucleic acid analyte from a sample, or a complement thereof. In some embodiments, the target nucleic acid analyte comprises a nucleic acid, optionally wherein the analyte is selected from genomic DNA, RNA, synthetic DNA, or synthetic RNA. In some embodiments, the target nucleic acid analyte is derived from a biological sample, optionally wherein the biological sample is derived from a human subject.

In some embodiments, the target nucleic acid analyte comprises an RNA selected from the group consisting of small interfering RNA (siRNA), microRNA (miRNA), P-element-induced wimpy testis (PIWI)-interacting RNA (piRNA), small nucleolar RNA (snoRNA), small nuclear RNA (snRNA), messenger RNA (mRNA), ribosomal RNA (rRNA), long non-coding RNAs (lncRNA), and transfer RNA (tRNA). In some embodiments, the RNA comprises mRNA.

In some embodiments, the target DNA molecule comprises a barcode. In some embodiments, the target DNA molecule comprises one or more of (i) a unique molecular identifier (UMI); (ii) a barcode; (iii) a sequence of at least a portion of a target analyte, or a complement thereof; and (iv) a capture sequence.

In some embodiments, the methods further comprise determining the nucleic acid sequence of the displaced DNA molecule. In some embodiments, determining the nucleic acid sequence comprises determining the sequence of one or more of
(i) a barcode or a complement thereof;
(ii) all or a portion of a target analyte or a complement thereof; and/or
(iii) a molecular identifier (UMI).

In some embodiments, the methods further comprise using the determined sequences of (i), (ii) and optionally (iii) to identify the abundance of the target nucleic acid analyte in a biological sample.

In some embodiments, the size of the first primer or second strand of the double stranded adapter is between about 8 and about 100 nucleotides, inclusive. In some embodiments, the second hybrid region comprises between about four and about 30 RNA nucleotides, inclusive. In some embodiments, the first region comprises between about four and about 30 DNA nucleotides, inclusive.

In some embodiments, the methods further comprise, after step (a), substantially removing unbound adapter from the target nucleic acid or first combined molecule, optionally wherein the removing comprises contacting the target nucleic acid or first combined molecule with a wash buffer.

In some embodiments, the target DNA molecule comprises a region complementary to a poly(A) nucleotide sequence, or a complement thereof. In some embodiments, the target DNA molecule comprises a nucleotide sequence of a coding region of an mRNA, or a complement thereof. In some embodiments, the target DNA molecule comprises one or more functional domains. In some embodiments, the functional domain comprises an amplification domain or a primer-binding site. In some embodiments, the target DNA molecule comprises one or more priming sites for a sequencing primer.

In some embodiments, the target DNA molecule is derived from a tissue sample. In some embodiments, the tissue sample is derived from a mammalian subject. In some embodiments, the subject is a human.

In some embodiments, target DNA molecule is from a fixed sample. In some embodiments, the fixed sample comprises a Formalin-Fixed Paraffin-Embedded (FFPE) sample. In some embodiments, the methods further comprise de-crosslinking the sample. In some embodiments, the methods further comprise deparaffinizing the tissue sample. In some embodiments, the methods further comprise staining and/or labelling the biological sample.

In some aspects, the present disclosure is related to primer sets comprising:
(a) a first primer comprising:
   (i) a first region, comprising a dsDNA strand displacing DNA sequence that is substantially complementary to a first primer binding site of a target nucleic acid, and
   (ii) a second hybrid region comprising one or more of RNA nucleotides, DNA nucleotides comprising a unique target sequence, or inosine nucleotides; and
(b) a second primer comprising the second hybrid region of the first primer.

In some embodiments, the second primer consists of the second hybrid region of the first primer. In some embodiments, the size of the first primer is between about 8 and about 100 nucleotides, inclusive. In some embodiments, the first primer comprises a first region comprising between about four and about 30 nucleotides, inclusive. In some embodiments, the first primer comprises a second region comprising between about four and about 30 nucleotides, inclusive.

In some embodiments, the first region comprises one or more modified nucleic acids. In some embodiments, the first region comprises one or more locked nucleic acids (LNAs). In some embodiments, the first region comprises one or more super G (8-aza-7-deazaguanosine) nucleic acids.

In some embodiments, the second region of the first primer comprises RNA nucleotides. In some embodiments, the second region of the first primer comprises inosine nucleotides. In some embodiments, the second region of the first primer comprises a unique target sequence. In some embodiments, the target sequence comprises a nickase recognition sequence. In some embodiments, the second region of the first primer comprises a methylated GATC nucleotide sequence.

In some aspects, the present disclosure is related to a kit comprising:
(a) a first primer comprising:
   (i) a first region, comprising a dsDNA strand displacing DNA sequence that is substantially complementary to a first primer binding site of a target nucleic acid, and
   (ii) a second hybrid region comprising one or more of RNA nucleotides, DNA nucleotides comprising a unique target sequence, or inosine nucleotides; and
(b) instructions for performing a method as described herein.

In some embodiments, kits include a first primer with a first region comprising one or more modified nucleic acids. In some embodiments, kits include a first primer with a first region comprising one or more locked nucleic acids (LNAs). In some embodiments, kits include a first primer with a first region comprising one or more super G (8-aza-7-deazaguanosine) nucleic acids.

In some embodiments, kits include a first primer with a second region comprising RNA nucleotides. In some embodiments, kits include a second primer comprising the second region of the first primer.

In some embodiments, kits of the present disclosure further comprise an RNA-specific nuclease enzyme. In some embodiments, the RNA-specific nuclease enzyme comprises RNaseH.

In some embodiments, kits comprise a first primer with a second region comprising inosine nucleotides. In some embodiments, kits comprise an endonuclease enzyme that degrades the inosine nucleotides. In some embodiments, kits comprise Endonuclease V.

In some embodiments, kits comprise a first primer with a second region comprising one or more unique target sequences. In some embodiments, kits comprise a nickase enzyme that selectively cleaves or removes one or more unique target sequences.

In some embodiments, kits comprise a first primer with a second region comprising one or more methylated GATC nucleotide sequences. In some embodiments, kits comprise a methylation-specific nuclease enzyme. In some embodiments, the methylation-specific nuclease enzyme comprises DpnI.

In some embodiments, kits comprise a single stranded adapter or a double stranded adapter. In some embodiments, the adapter comprises bases complementary to the second region of the first primer. In some embodiments, the first adapter comprises a 5′phosphate. In some embodiments, the kit comprises a ligase enzyme, such as a T4 ligase.

In some embodiments, kits comprise a strand displacing DNA polymerase. In some embodiments, the strand-displacing polymerase is Bst2.0, Bst3.0, Bsu or Phil29.

In some embodiments, kits comprise one or more of:
(a) a solvent suitable to solubilize unbound first primer(s) and/or unbound second primer(s);
(b) one or more DNA-specific Polymerase enzymes;
(c) one or more nuclease enzymes;
(d) buffers and/or reagents suitable for enzyme reactions according to the methods described herein.

In some aspects, the present disclosure is related to a partially double stranded nucleic acid adapter, comprising:
(I) a first adapter strand, comprising:
   (i) a first region, comprising DNA nucleotides; and
   (ii) a second hybrid 5′ overhang region, comprising RNA nucleotides, or DNA nucleotides comprising a unique target sequence, or inosine nucleotides; and
(II) a second adapter strand comprising a 3′ overhang DNA splint sequence, wherein the splint sequence is complementary to all or part of the first adapter strand and complementary to 5′ end of a target dsDNA molecule.

In some embodiments, the first adapter strand comprises between about 8 and about 100 nucleotides, inclusive. In some embodiments, the first region of the first adapter strand comprises between about four and about 30 nucleotides, inclusive. In some embodiments, the first region of the second adapter strand comprises between about four and about 30 nucleotides, inclusive. In some embodiments, the second hybrid 5′ overhang region comprises between about four and about 30 nucleotides, inclusive. In some embodiments, the second hybrid 5′ overhang region comprises RNA nucleotides. In some embodiments, the second hybrid 5′ overhang region comprises inosine nucleotides. In some embodiments, the second hybrid 5′ overhang region comprises a unique target sequence, and the target sequence comprises a nickase recognition sequence. In some embodiments, the second hybrid region comprises a methylated GATC nucleotide sequence.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1A shows an exemplary sandwiching process where a first substrate (e.g., a slide), including a biological sample, and a second substrate (e.g., array slide) are brought into proximity with one another.

FIG. 1B shows a fully formed sandwich configuration creating a chamber formed from the one or more spacers, the first substrate, and the second substrate.

FIG. 2A shows a perspective view of an exemplary sample handling apparatus in a closed position.

FIG. 2B shows a perspective view of an exemplary sample handling apparatus in an open position.

FIG. 3A shows the first substrate angled over (superior to) the second substrate.

FIG. 3B shows that as the first substrate lowers, and/or as the second substrate rises, the dropped side of the first substrate may contact a drop of reagent medium.

FIG. 3C shows a full closure of the sandwich between the first substrate and the second substrate with one or more spacers contacting both the first substrate and the second substrate.

FIG. 4A shows a side view of the angled closure workflow.

FIG. 4B shows a top view of the angled closure workflow.

FIG. 5 is a schematic diagram showing an example of a barcoded capture probe, as described herein.

FIG. 6 shows a schematic illustrating a cleavable capture probe.

FIG. 7 shows exemplary capture domains on capture probes.

FIG. 8 shows an exemplary arrangement of barcoded features within an array.

FIG. 9A shows an exemplary workflow for performing templated capture and producing a ligation product, and FIG. 9B shows an exemplary workflow for capturing a ligation product from FIG. 9A on a substrate.

FIG. 10 is a schematic diagram of an exemplary analyte capture agent.

FIG. 11 is a schematic diagram depicting an exemplary interaction between a feature-immobilized capture probe 1124 and an analyte capture agent 1126.

FIG. 12A is a schematic diagram depicting the components of an exemplary target nucleic acid (12400) attached to a substrate (12300). A target nucleic acid can be formed from the capture of an exemplary probe (12100) onto an exemplary array oligo (12200). An exemplary probe (12100) includes a first priming site (such as a priming site for a sequencing primer, e.g., a Read 2S domain) (1250), Left-hand side ligation domain (1260) and Right-hand side ligation domain (1270) and a capture sequence that is complementary to a capture domain of a capture probe (such as a Poly(A) capture tag domain) (1280). An exemplary array oligo (12200) includes a capture domain (1210), unique molecular identifier (1220), spatial barcode domain (1230) and a second priming site (such as a priming site for a second sequencing primer, e.g., a Read 1T domain) (1240) at 5′ region. In some forms, 5′ region is conjugated with a substrate (12300). Typically, a target nucleic acid (12400) is formed following annealing of the probe with the array oligo by subsequent polymerase-based extension and denaturation to yield the target nucleic acid including a complement of the first priming site (1205), a complement of the Left hand side ligation domain (1206), a complement of the Right hand side ligation domain (1207), a complement of the capture sequence (1280), as well as a unique molecular identifier (1220), spatial barcode domain (1230) and second priming site (1240). In the schematic, the target nucleic acid (12400) is attached to a substrate (12300).

FIG. 12B is a schematic diagram of an exemplary work-flow for amplification of a target nucleic acid (12400) as depicted in FIG. 12A, showing components of RNase-mediated Strand Displacement Amplification (RH-SDA), including Primer 1 (12500) having a 5′ RNA domain (1290), and a 3′ DNA domain complementary to all or part of the complement of the first priming site of the target nucleic acid (1295); and a strand displacing DNA polymerase (12700). Amplification is initiated by contacting the target nucleic acid (12400), which preferably includes DNA, with Primer 1 (12500), which hybridizes to the complement of the first priming site (1205) of the target nucleic acid, and the strand displacing DNA polymerase (12700), which drives the replication of a new second strand using the target nucleic acid as a template. The new second strand is complementary to the target nucleic acid (i.e., includes sequences complementary to a first priming site (1295), a Left hand side ligation domain (12061), a Right hand side ligation domain (12071), a complement of a capture domain (12101), a complement of a unique molecular identifier (12201), a complement of a spatial barcode domain (12301) and a complement of a second priming site (12401), respectively. The strand displacing DNA polymerase (12700) can also drive extension of the target nucleic acid (12400) using the first primer as a template to generate a complement of 5′ RNA domain of Primer 1 (1209), which together with the new second strand provides a double-stranded (ds) extension product (12900) of the target.

FIG. 12C is a schematic diagram of an exemplary work-flow for amplification of the double-stranded extension product (12900) of the target, as depicted in FIG. 12B, showing RNase (12800) mediated digestion of the RNA component, i.e. 5′ RNA domain (1290) of Primer 1 to form a truncated ds extended product (12910). Strand displacement proceeds by contacting the truncated ds extended product (12910) with Primer 2 (12600) including a single RNA domain (1296) that constitutes the same RNA sequence as 5′ RNA domain (1290) of Primer 1, and which binds the complementary sequence domain (1209) to form a target hybridized with primer 2 (12920), together with a strand displacing DNA polymerase (12700).

FIG. 12D is a schematic diagram of an exemplary work-flow depicting a strand displacement of the target hybridized with primer 2 (12920 of FIG. 12C) by strand displacing DNA polymerase (12700), which creates a new strand by extending primer 2 (1296) using the bottom strand of the target hybridized with primer 2 (12920) as a template. The reaction proceeds to produce an entire displaced strand (12930) that is complementary to the initial target nucleic acid, as well as a double-stranded extension product (12900) of the target.

FIG. 12E is a schematic diagram of an exemplary work-flow depicting the process for repeated amplification, by repeating the RNase mediated digestion (as depicted in FIG. 12C). The figure shows RNase (12800) digestion of the RNA component (1296) of the double-stranded extension product (12900), to form a truncated ds extended product (12910), to which primer 2 hybridizes and is subsequently extended by a strand displacing DNA polymerase.

FIGS. 13A-13C are schematic diagrams depicting three alternative designs for adapter handles for use in the described methods for amplification of a double stranded (ds) target nucleic acid. FIG. 13A shows a partially double stranded adapter handle (13100) including a first strand having a single 5′-3′ DNA domain (1351) and a second strand having two DNA domains, including a first domain at the 5′ end (1315), that is complementary to, and hybridized with (1351), and a second DNA domain at 3′ end (1310), that is complementary to a sequence of a first primer, and which forms a 3′ overhang. The 5′ end of the second strand is depicted as including a 5′ phosphate moiety. FIG. 13B shows a double stranded (ds) adapter handle (13200) including a first strand having two domains, including an RNA domain at the 5′ end (1301), and a DNA domain at 3′ end (1351); and a second strand having two DNA domains, including a first DNA domain at the 5′ end (1315), that is complementary to, and hybridized with the DNA domain of the first strand (1351), and a second DNA domain at 3′ end (1310), that is complementary to, and hybridized with the RNA domain of the first strand (1301). The 5′ end of the second strand is depicted as including a 5′ phosphate moiety. FIG. 13C shows a double stranded (ds) adapter handle (13300) including a first strand having one RNA domain (1320); and a second strand having two DNA domains, including a first DNA domain at the 5′ end (1315), that is complementary to, and hybridized with a portion of the RNA domain of the first strand (1320), and a second DNA domain at 3′ end (1310), that is complementary to, and hybridized with another portion of the RNA domain of the first strand (1320). The 5′ end of the second strand is depicted as including a 5′ phosphate moiety.

FIG. 13D is a schematic diagram of an exemplary work-flow for amplification of a double-stranded (ds) target nucleic acid (13000). The ds target nucleic acid includes a first strand as depicted in FIG. 12A, which can be attached at the 5′ end to a substrate (12300), including a domain that is a complement of a Read 2S domain (1205), a domain that is a complement of a Left-hand side RNA templated ligation domain (1206), a domain that is a complement of a Right-hand side RNA templated ligation domain (1207), a capture domain (1210), unique molecular identifier (1220), spatial barcode domain (1230) and Read 1T domain (1240) at 5′ region. The ds target nucleic acid also includes a second strand, having domains complementary to those of the first strand, including a Read 2S domain (1295), a Left hand side RNA templated ligation domain (12061), a Right hand side RNA templated ligation domain (12071), a complement of a capture domain (12101), a complement of a unique molecular identifier (12201), a complement of a spatial barcode domain (12301) and a complement of a Read 1T domain (12401). The amplification first includes addition of an adapter handle (13200) to the free 3′ end of the ds target nucleic acid. In some forms, the addition includes ligation via a 5′ phosphate moiety included in the adapter.

FIG. 13E is a schematic diagram of an exemplary work-flow for amplification of an adapter-bound double-stranded (ds) target nucleic acid (13010), including an adapter handle (13200) bound to a (ds) target nucleic acid (13000) depicted in FIG. 13D. The amplification includes digestion of the RNA component (1301) of the adapter by an RNase (12800), e.g., RNase H, to form a truncated adapter-bound ds target (13220) that includes a 3′ overhang corresponding to the second DNA domain at 3′ end (1310) of the second strand of the ds adapter handle.

FIG. 13F is a schematic diagram of an exemplary work-flow depicting contacting the truncated adapter-bound ds target (13220) of FIG. 13E with Primer 2 (12600) including a single RNA domain (1296) that constitutes the same RNA sequence as the 5′ RNA domain (1301) of the ds adapter handle, and which hybridizes to a complementary domain (1310) of the truncated adapter-bound ds target to form a target hybridized with primer 2 (1330), together with a strand displacing DNA polymerase (12700).

FIG. 13G is a schematic diagram of an exemplary work-flow depicting strand displacement by amplification of the target hybridized with primer 2 depicted in FIG. 13F (1330) through extension of primer 2 using a strand displacing polymerase (1340). The reaction proceeds to displace an entire strand (1350) that is complementary to the first strand of the target nucleic acid and includes the DNA domain at the 3′ end (1351) of the first strand of the adapter handle, and provides a copy of the adapter-bound double-stranded (ds) target nucleic acid (13010) depicted in FIG. 13E.

FIG. 13H is a schematic diagram of an exemplary work-flow depicting the process for repeating the amplification, by repeating the digestion of the RNA component (1310) of the adapter with RNase (12800) to form a truncated adapter-bound ds target (1320) that includes a 3′ overhang corresponding to the second DNA domain at 3′ end (1310) of the adapter (as depicted in FIG. 13E), and then contacting the truncated adapter-bound ds target (1320) of FIG. 13E with Primer 2 (12600) including a single RNA domain (1296) that constitutes the same RNA sequence as the 5′ RNA domain (1301) of the adapter, and which hybridizes to the complementary sequence domain (1310) of the adapter to form a target hybridized with primer 2 (1330), together with a strand displacing DNA polymerase (12700), to repeat the displacement/amplification depicted in FIGS. 13F and 13G.

FIG. 14 shows an example strand displacement amplification workflow using a strand displacing first DNA/RNA hybrid primer for binding and displacing a DNA strand of a target dsDNA molecule, an RNase, a second DNA/RNA hybrid primer, and a strand displacing polymerase.

FIG. 15 shows an example strand displacement amplification workflow using a partially double stranded DNA/RNA hybrid adapter including a splint sequence for linking to a target dsDNA molecule, an RNase, a second DNA/RNA hybrid primer, and a strand displacing polymerase.

### DETAILED DESCRIPTION

### I. Spatial Analysis Methods

Spatial analysis methodologies described herein can provide a vast amount of analyte and/or expression data for a variety of analytes within a biological sample at high spatial resolution, while retaining native spatial context. Spatial analysis methods can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 11,447,807, 11,352,667, 11,168,350, 11,104,936, 11,008,608, 10,995,361, 10,913,975, 10,774,374, 10,724,078, 10,640,816, 10,494,662, 10,480,022, 10,364,457, 10,317,321, 10,059,990, 10,041,949, 10,030,261, 10,002,316, 9,879,313, 9,783,841, 9,727,810, 9,593,365, 8,951,726, 8,604,182, and 7,709,198; U.S. Patent Application Publication Nos. 2020/0239946, 2020/0080136, 2020/0277663, 2019/0330617, 2020/0256867, 2020/0224244, 2019/0085383, and 2013/0171621; PCT Publication Nos. WO2018/091676, WO2020/176788, WO2017/144338, and WO2016/057552; Non-patent literature references Rodriques et al., Science 363 (6434): 1463-1467, 2019; Lee et al., Nat. Protoc. 10 (3): 442-458, 2015; Trejo et al., PLOS ONE 14 (2): e0212031, 2019; Chen et al., Science 348 (6233): aaa6090, 2015; Gao et al., BMC Biol. 15:50, 2017; and Gupta et al., Nature Biotechnol. 36:1197-1202, 2018; and the Visium Spatial Gene Expression Reagent Kits User Guide (e.g., Rev F, dated January 2022) and/or the Visium Spatial Gene Expression Reagent Kits-Tissue Optimization User Guide (e.g., Rev E, dated February 2022), both of which are available at the 10× Genomics Support Documentation website, and can be used herein in any combination, and each of which is incorporated herein by reference in its entirety. Further non-limiting aspects of spatial analysis methodologies and compositions are described herein.

Some general terminology that may be used in this disclosure can be found in Section (I) (b) of PCT Publication No. WO2020/176788 and/or U.S. Patent Application Publication No. 2020/0277663, which is herein incorporated by reference. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I) (c) of PCT Publication No. WO2020/176788 and/or U.S. Patent Application Publication No. 2020/0277663, which is herein incorporated by reference. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, the biological sample is a tissue sample. In some embodiments, the biological sample (e.g., tissue sample) is a tissue microarray (TMA). A tissue microarray contains multiple representative tissue samples—which can be from different tissues or organisms—assembled on a single histologic slide. The TMA can therefore allow for high throughput analysis of multiple specimens at the same time. Tissue microarrays may be paraffin blocks produced by extracting cylindrical tissue cores from different paraffin donor blocks and re-embedding these tissue cores into a single recipient (microarray) block at defined array coordinates.

The biological sample as used herein can be any suitable biological sample described herein or known in the art. In some embodiments, the biological sample is a tissue sample. In some embodiments, the tissue sample is a solid tissue sample. In some embodiments, the biological sample is a tissue section (e.g., a fixed tissue section). In some embodiments, the tissue is flash-frozen and sectioned. Any suitable method described herein or known in the art can be used to flash-freeze and section the tissue sample. In some embodiments, the biological sample, e.g., the tissue, is flash-frozen using liquid nitrogen before sectioning. In some embodiments, the biological sample, e.g., a tissue sample, is flash-frozen using nitrogen (e.g., liquid nitrogen), isopentane, or hexane.

In some embodiments, the biological sample, e.g., the tissue, is embedded in a matrix e.g., optimal cutting temperature (OCT) compound to facilitate sectioning. OCT compound is a formulation of clear, water-soluble glycols and resins, providing a solid matrix to encapsulate biological (e.g., tissue) specimens. In some embodiments, the sectioning is performed by cryosectioning, for example using a microtome. In some embodiments, the methods further comprise a thawing step, after the cryosectioning.

The biological sample can be from a mammal. In some instances, the biological sample is from a human, mouse, or rat. In addition to the subjects described above, the biological sample can be obtained from non-mammalian organisms (e.g., a plant, an insect, an arachnid, a nematode (e.g., Caenorhabditis elegans), a fungus, an amphibian, or a fish (e.g., zebrafish)). A biological sample can be obtained from a prokaryote such as a bacterium, e.g., Escherichia coli, Staphylococci or Mycoplasma pneumoniae; an archacon; a virus such as Hepatitis C virus or human immunodeficiency virus; or a viroid. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

In some embodiments, the biological sample, e.g., the tissue sample, is fixed in a fixative including alcohol, for example, methanol. In some embodiments, instead of methanol, acetone or an acetone-methanol mixture can be used. In some embodiments, the fixation is performed after sectioning. In some instances, when the biological sample is fixed using a fixative including an alcohol (e.g., methanol or acetone-methanol mixture), the biological sample is not decrosslinked afterward. In some preferred embodiments, the biological sample is fixed using a fixative including an alcohol (e.g., methanol or an acetone-methanol mixture) after freezing and/or sectioning. In some instances, the biological sample is flash-frozen, and then the biological sample is sectioned and fixed (e.g., using methanol, acetone, or an acetone-methanol mixture). In some instances when methanol, acetone, or an acetone-methanol mixture is used to fix the biological sample, the sample is not decrosslinked at a later step. In instances when the biological sample is frozen (e.g., flash frozen using liquid nitrogen and embedded in OCT) followed by sectioning and alcohol (e.g., methanol, acetone-methanol) fixation or acetone fixation, the biological sample is referred to as “fresh frozen”. In some embodiments, fixation of the biological sample, e.g., using acetone and/or alcohol (e.g., methanol, acetone-methanol), is performed while the sample is mounted on a substrate (e.g., glass slide, such as a positively charged glass slide).

In some embodiments, the biological sample, e.g., the tissue sample, is fixed e.g., immediately after being harvested from a subject. In such embodiments, the fixative is preferably an aldehyde fixative, such as paraformaldehyde (PFA) or formalin. In some embodiments, the fixative induces crosslinks within the biological sample. In some embodiments, after fixing, e.g., by formalin or PFA, the biological sample is dehydrated via sucrose gradient. In some instances, the fixed biological sample is treated with a sucrose gradient and then embedded in a matrix, e.g., OCT compound. In some instances, the fixed biological sample is not treated with a sucrose gradient, but rather is embedded in a matrix, e.g., OCT compound after fixation. In some embodiments when a fixed frozen tissue sample is treated with a sucrose gradient, the sample can be rehydrated using an ethanol gradient. In some embodiments, the PFA or formalin fixed biological sample, which can be optionally dehydrated via sucrose gradient and/or embedded in OCT compound, is then frozen, e.g., for storage or shipment. In such instances, the biological sample is referred to as “fixed frozen”. In preferred embodiments, a fixed frozen biological sample is not treated with methanol. In preferred embodiments, a fixed frozen biological sample is not paraffin embedded. Thus, in preferred embodiments, a fixed frozen biological sample is not deparaffinized. In some embodiments, a fixed frozen biological sample is rehydrated using an ethanol gradient.

In some instances, the biological sample (e.g., a fixed frozen tissue sample) is treated with a citrate buffer. Citrate buffer can be used to de-crosslink antigens and fixation medium for antigen retrieval in the biological sample. Thus, any suitable decrosslinking agent can be used in addition, or alternatively, to citrate buffer. In some embodiments, for example, the biological sample (e.g., a fixed frozen tissue sample) is decrosslinked using TE buffer.

In any of the foregoing, the biological sample can further be stained, imaged, and/or destained. For example, in some embodiments, a fresh frozen tissue sample or fixed frozen tissue sample is stained (e.g., via cosin and/or hematoxylin), imaged, destained (e.g., via HCl), or a combination thereof. In some embodiments, when a fresh frozen tissue sample is fixed in methanol, the sample is treated with isopropanol prior to being stained (e.g., via cosin and/or hematoxylin), imaged, destained (e.g., via HCl), or a combination thereof. In some embodiments when a fixed frozen tissue sample is treated with a sucrose gradient, the sample can be rehydrated using an ethanol gradient before being stained, (e.g., via cosin and/or hematoxylin), imaged, destained (e.g., via HCl), decrosslinked (e.g., via TE buffer or citrate buffer), or a combination thereof. In some embodiments, the biological sample can undergo further fixation (e.g., while mounted on a substrate), stained, imaged, and/or destained. For example, a fixed frozen biological sample may be subject to an additional fixing step (e.g., using PFA) before optional ethanol rehydration, staining, imaging, and/or destaining.

In any of the foregoing, the biological sample can be fixed using PAXGENE®. For example, the biological sample can be fixed using PAXGENE® in addition, or alternatively to, a fixative disclosed herein or known in the art (e.g., alcohol, acetone, acetone-alcohol, formalin, paraformaldehyde). PAXGENE® is a non-cross-linking mixture of different alcohols, an acid, and a soluble organic compound that preserves morphology and biomolecules. PAXGENE® provides a two-reagent fixative system in which tissue is firstly fixed in a solution containing methanol and acetic acid, then stabilized in a solution containing ethanol. See, Ergin B. et al., J Proteome Res. 2010 Oct. 1; 9 (10): 5188-96; Kap M. et al., PLOS One.; 6 (11): e27704 (2011); and Mathieson W. et al., Am J Clin Pathol.; 146 (1): 25-40 (2016), each of which is hereby incorporated by reference in its entirety, for a description and evaluation of PAXGENE® for tissue fixation. Thus, in some embodiments, when the biological sample, e.g., the tissue sample, is fixed in a fixative including alcohol, the fixative is PAXGENE®. In some embodiments, a fresh frozen tissue sample is fixed with PAXGENE®. In some embodiments, a fixed frozen tissue sample is fixed with PAXGENE®.

In some embodiments, the biological sample, e.g., the tissue sample, is fixed, for example in methanol, acetone, acetone-methanol, PFA, PAXGENE®, or is formalin-fixed and paraffin-embedded (FFPE). In some embodiments, the biological sample comprises intact cells. In some embodiments, the biological sample is a cell pellet, e.g., a fixed cell pellet, e.g., an FFPE cell pellet. FFPE samples are used in some instances in the RNA-templated ligation (RTL) methods disclosed herein. A limitation of direct RNA capture for fixed samples is that the RNA integrity of fixed (e.g., FFPE) samples can be lower than of a fresh sample, thereby capturing RNA directly from fixed samples, e.g., by capture of a common sequence such as a poly(A) tail of an mRNA molecule, can be more difficult. By utilizing RTL probes that hybridize to RNA target sequences in the transcriptome, RNA analytes can be captured without requiring that both a poly(A) tail and target sequences remain intact. Accordingly, RTL probes can be utilized to beneficially improve capture and spatial analysis of fixed samples. The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to destaining the sample. In some embodiments, the biological sample is stained using an H&E staining method. In some embodiments, the tissue sample is stained and imaged for about 10 minutes to about 2 hours (or any of the subranges of this range described herein). Additional time may be needed for staining and imaging of different types of biological samples.

The tissue sample can be obtained from any suitable location in a tissue or organ of a subject, e.g., a human subject. In some instances, the sample is a mouse sample. In some instances, the sample is a human sample. In some embodiments, the sample can be derived from skin, brain, breast, lung, liver, kidney, prostate, tonsil, thymus, testes, bone, lymph node, ovary, eye, heart, or spleen. In some instances, the sample is a human or mouse breast tissue sample. In some instances, the sample is a human or mouse brain tissue sample. In some instances, the sample is a human or mouse lung tissue sample. In some instances, the sample is a human or mouse tonsil tissue sample. In some instances, the sample is a human or mouse liver tissue sample. In some instances, the sample is a human or mouse bone, skin, kidney, thymus, testes, or prostate tissue sample. In some embodiments, the tissue sample is derived from normal or diseased tissue. In some embodiments, the sample is an embryo sample. The embryo sample can be a non-human embryo sample. In some instances, the sample is a mouse embryo sample.

Biological samples are also described in Section (I) (d) of PCT Publication No. WO2020/176788 and/or U.S. Patent Application Publication No. 2020/0277663, which is herein incorporated by reference.

The following embodiments can be used with any of the methods described herein. In some embodiments, the biological sample (e.g., a fixed and/or stained biological sample) is imaged. In some embodiments, the biological sample is visualized or imaged using bright field microscopy. In some embodiments, the biological sample is visualized or imaged using fluorescence microscopy. The biological sample can be visualized or imaged using additional methods of visualization and imaging known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy. In some embodiments, the sample is stained and imaged prior to adding reagents for analyzing captured analytes, as disclosed herein, to the biological sample.

In some embodiments, the methods include staining the biological sample. In some embodiments, the staining includes the use of hematoxylin and/or cosin. Non-limiting examples of stains include histological stains (e.g., hematoxylin and/or cosin) and immunological stains (e.g., fluorescent stains). In some embodiments, a biological sample can be stained using any number of biological stains, including but not limited to, acridine orange, Bismarck brown, carmine, coomassie blue, cresyl violet, DAPI (4′,6-diamidino-2-phenylindole), cosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the biological sample can be stained using known staining techniques, including Can-Grunwald, Giemsa, hematoxylin and cosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the staining includes the use of a detectable label, such as a radioisotope, a fluorophore, a chemiluminescent compound, a bioluminescent compound, or a combination thereof.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of PCT Publication No. WO2020/176788 and/or U.S. Patent Application Publication No. 2020/0277663, which is herein incorporated by reference. Briefly, any of the methods described herein includes permeabilizing the biological sample. For example, the biological sample can be permeabilized to facilitate transfer of extension products to the capture probes on the array. In some embodiments, the permeabilizing includes the use of an organic solvent (e.g., acetone, ethanol, or methanol), a detergent (e.g., saponin, Triton X-100™, Tween-20™, or sodium dodecyl sulfate (SDS)), an enzyme (e.g., an endopeptidase, an exopeptidase, or a protease), or a combination thereof. In some embodiments, the permeabilizing includes the use of an endopeptidase, a protease, SDS, polyethylene glycol tert-octylphenyl ether, polysorbate 80, polysorbate 20, N-lauroylsarcosine sodium salt solution, saponin, TRITON X-100™, TWEEN-20™, or a combination thereof. In some embodiments, the endopeptidase is pepsin. In some embodiments, the endopeptidase is Proteinase K. Additional methods for sample permeabilization are described, for example, in Jamur et al., Method Mol. Biol. 588:63-66, 2010, which is herein incorporated by reference.

Array-based spatial analysis methods can involve the transfer of one or more analytes or derivatives thereof from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI) and a capture domain). In some instances, the capture probe includes a homopolymer sequence, such as a poly(T) sequence. In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of PCT Publication No. WO2020/176788 and/or U.S. Patent Application Publication No. 2020/0277663, which is herein incorporated by reference. Generation of capture probes can be achieved by any appropriate method, including those described in Section (II)(d)(ii) of PCT Publication No. WO2020/176788 and/or U.S. Patent Application Publication No. 2020/0277663, which is herein incorporated by reference.

In some instances, a capture probe and a nucleic acid analyte interaction (or any other nucleic acid to nucleic acid interaction) occurs because the sequences of the two nucleic acids are substantially complementary to one another. By “substantial,” “substantially,” and the like, two nucleic acid sequences can be complementary when at least 60% of the nucleotide residues of one nucleic acid sequence are complementary to nucleotide residues of the other nucleic acid sequence. The complementary residues within a particular complementary nucleic acid sequence need not always be contiguous with each other, but can be interrupted by one or more non-complementary residues within the complementary nucleic acid sequence. In some embodiments, at least 60%, but less than 100%, of the residues of one of the two complementary nucleic acid sequences are complementary to residues of the other nucleic acid sequence. In some embodiments, at least 70%, 80%, 90%, 95%, or 99% of the residues of one nucleic acid sequence are complementary to residues of the other nucleic acid sequence. Sequences are said to be “substantially complementary” when at least 60% (e.g., at least 70%, at least 80%, or at least 90%) of the residues of one nucleic acid sequence are complementary to residues of the other nucleic acid sequence. In some embodiments, the biological sample is mounted on a first substrate and the substrate comprising the array of capture probes is a second substrate. In this configuration, one or more analytes or analyte derivatives (e.g., intermediate agents; e.g., ligation products) are then released from the biological sample and migrate to the second substrate comprising an array of capture probes. In some embodiments, the release and migration of the analytes or analyte derivatives to the second substrate comprising the array of capture probes occurs in a manner that preserves the original spatial context of the analytes in the biological sample. This method can be referred to as a sandwiching process, which is described, e.g., in U.S. Patent Application Pub. No. 2021/0189475 and PCT Pub. Nos. WO 2021/252747 A1, WO 2022/061152 A2, and WO 2022/140028 A1, each of which is herein incorporated by reference.

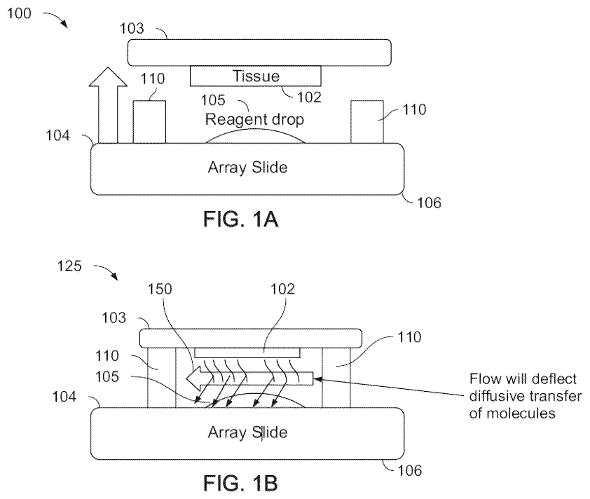

FIG. 1A shows an exemplary sandwiching process 100 where a first substrate (e.g., slide 103), including a biological sample 102, and a second substrate (e.g., array slide 104 including an array having spatially barcoded capture probes 106) are brought into proximity with one another. As shown in FIG. 1A, a liquid reagent drop (e.g., permeabilization solution 105) is introduced on the second substrate in proximity to the capture probes 106 and in between the biological sample 102 and the second substrate (e.g., slide 104 including an array having spatially barcoded capture probes 106). The permeabilization solution 105 may release analytes or analyte derivatives (e.g., intermediate agents; e.g., ligation products) that can be captured by the capture probes of the array 106.

During the exemplary sandwiching process, the first substrate is aligned with the second substrate, such that at least a portion of the biological sample is aligned with at least a portion of the capture probes (e.g., aligned in a sandwich configuration). As shown, the second substrate (e.g., array slide 104) is in an inferior position to the first substrate (e.g., slide 103). In some embodiments, the first substrate (e.g., slide 103) may be positioned superior to the second substrate (e.g., slide 104). A reagent medium 105 within a gap between the first substrate (e.g., slide 103) and the second substrate (e.g., slide 104) creates a liquid interface between the two substrates. The reagent medium may be a permeabilization solution which permeabilizes and/or digests the biological sample 102. In some embodiments wherein the biological sample 102 has been pre-permeabilized, the reagent medium is not a permeabilization solution. Herein, the reagent medium may also comprise one or more of a monovalent salt, a divalent salt, ethylene carbonate, and/or glycerol. In some embodiments, analytes (e.g., mRNA transcripts) and/or analyte derivatives (e.g., intermediate agents; e.g., ligation products) of the biological sample 102 may release from the biological sample, and actively or passively migrate (e.g., diffuse) across the gap toward the capture probes on the array 106. Alternatively, in certain embodiments, migration of the analyte or analyte derivative (e.g., intermediate agent; e.g., ligation product) from the biological sample is performed actively (e.g., electrophoretic, by applying an electric field to promote migration). Exemplary methods of electrophoretic migration are described in WO 2020/176788 and U.S. Patent Application Pub. No. 2021/0189475, each of which is hereby incorporated by reference in its entirety.

As further shown, one or more spacers 110 may be positioned between the first substrate (e.g., slide 103) and the second substrate (e.g., array slide 104 including spatially barcoded capture probes 106). The one or more spacers 110 may be configured to maintain a separation distance between the first substrate and the second substrate. While the one or more spacers 110 is shown as disposed on the second substrate, the spacer may additionally or alternatively be disposed on the first substrate.

In some embodiments, the one or more spacers 110 is configured to maintain a separation distance between first and second substrates that is between about 2 microns (μm) and about 1 mm (e.g., between about 2 μm and about 800 μm, between about 2 μm and about 700 μm, between about 2 μm and about 600 μm, between about 2 μm and about 500 μm, between about 2 μm and about 400 μm, between about 2 μm and about 300 μm, between about 2 μm and about 200 μm, between about 2 μm and about 100 μm, between about 2 μm and about 25 μm, or between about 2 μm and about 10 μm), measured in a direction orthogonal to the surface of first substrate that supports the biological sample. In some instances, the separation distance is about 2, 3, 4, 5, 6, 7, 8, 9, 10, 11, 12, 13, 14, 15, 16, 17, 18, 19, 20, 21, 22, 23, 24, or 25 μm. In some embodiments, the separation distance is less than 50 μm. In some embodiments, the separation distance is less than 25 μm. In some embodiments, the separation distance is less than 20 μm. The separation distance may include a distance of at least 2 μm.

FIG. 1B shows a fully formed sandwich configuration 125 creating a chamber 150 formed from the one or more spacers 110, the first substrate (e.g., the slide 103), and the second substrate (e.g., the slide 104 including an array 106 having spatially barcoded capture probes) in accordance with some example implementations. In the example of FIG. 1B, the liquid reagent (e.g., the permeabilization solution 105) fills the volume of the chamber 150 and may create a permeabilization buffer that allows analytes (e.g., mRNA transcripts and/or other molecules) or analyte derivatives (e.g., intermediate agents; e.g., ligation products) to diffuse from the biological sample 102 toward the capture probes of the second substrate (e.g., slide 104). In some aspects, flow of the permeabilization buffer may deflect transcripts and/or molecules from the biological sample 102 and may affect diffusive transfer of analytes or analyte derivatives (e.g., intermediate agents; e.g., ligation products) for spatial analysis. A partially or fully sealed chamber 150 resulting from the one or more spacers 110, the first substrate (e.g., slide 103), and the second substrate (e.g., slide 104) may reduce or prevent undesirable movement (e.g., convective movement) of transcripts and/or molecules during the diffusive transfer from the biological sample 102 to the capture probes.

The sandwiching process methods described above can be implemented using a variety of hardware components. For example, the sandwiching process methods can be implemented using a sample holder (also referred to herein as a support device, a sample handling apparatus, and an array alignment device). Further details on support devices, sample holders, sample handling apparatuses, or systems for implementing a sandwiching process are described in, e.g., U.S. Patent Application Pub. No. 2021/0189475 and PCT Publ. No. WO 2022/061152 A2, each of which is incorporated by reference in its entirety.

In some embodiments of a sample holder, the sample holder can include a first member including a first retaining mechanism configured to retain a first substrate comprising a biological sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane of the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

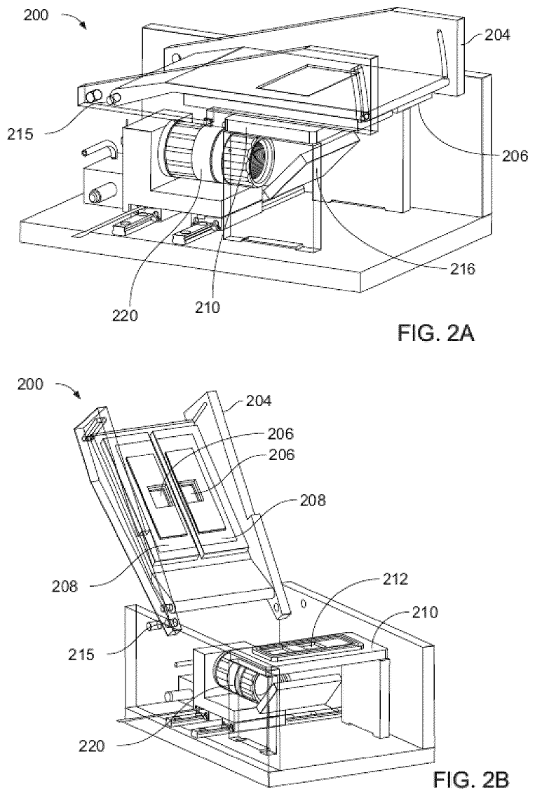

FIG. 2A is a perspective view of an example sample handling apparatus 200 in a closed position in accordance with some example implementations. As shown, the sample handling apparatus 200 includes a first member 204, a second member 210, optionally an image capture device 220, a first substrate 206, optionally a hinge 215, and optionally a mirror 216. The hinge 215 may be configured to allow the first member 204 to be positioned in an open or closed configuration by opening and/or closing the first member 204 in a clamshell manner along the hinge 215.

FIG. 2B is a perspective view of the example sample handling apparatus 200 in an open position in accordance with some example implementations. As shown, the sample handling apparatus 200 includes one or more first retaining mechanisms 208 configured to retain one or more first substrates 206. In the example of FIG. 2B, the first member 204 is configured to retain two first substrates 206, however the first member 204 may be configured to retain more or fewer first substrates 206.

In some aspects, when the sample handling apparatus 200 is in an open position (e.g., in FIG. 2B), the first substrate 206 and/or the second substrate 212 may be loaded and positioned within the sample handling apparatus 200 such as within the first member 204 and the second member 210, respectively. As noted, the hinge 215 may allow the first member 204 to close over the second member 210 and form a sandwich configuration.

In some aspects, after the first member 204 closes over the second member 210, an adjustment mechanism of the sample handling apparatus 200 may actuate the first member 204 and/or the second member 210 to form the sandwich configuration for the permeabilization step (e.g., bringing the first substrate 206 and the second substrate 212 closer to each other and within a threshold distance for the sandwich configuration). The adjustment mechanism may be configured to control a speed, an angle, a force, or the like of the sandwich configuration.

In some embodiments, the biological sample (e.g., sample 102 from FIG. 1A) may be aligned within the first member 204 (e.g., via the first retaining mechanism 208) prior to closing the first member 204 such that a desired region of interest of the sample is aligned with the barcoded array of the second substrate (e.g., the slide 104 from FIG. 1A), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 206 and/or the second substrate 212 to maintain a minimum spacing between the first substrate 206 and the second substrate 212 during sandwiching. In some aspects, the permeabilization solution (e.g., permeabilization solution 305) may be applied to the first substrate 206 and/or the second substrate 212. The first member 204 may then close over the second member 210 and form the sandwich configuration. Analytes or analyte derivatives (e.g., intermediate agents; e.g., ligation products) may be captured by the capture probes of the array and may be processed for spatial analysis.

In some embodiments, during the permeabilization step, the image capture device 220 may capture images of the overlap area between the biological sample and the capture probes on the array 106. If more than one first substrates 206 and/or second substrates 212 are present within the sample handling apparatus 200, the image capture device 220 may be configured to capture one or more images of one or more overlap areas.

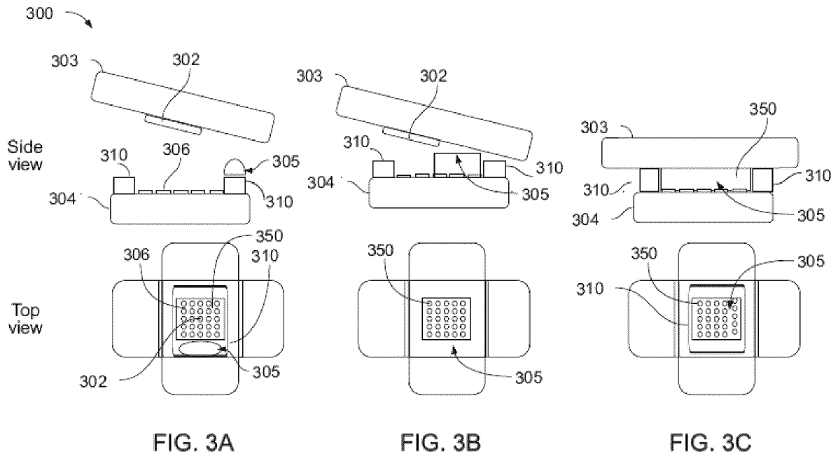

Provided herein are methods for delivering a fluid to a biological sample disposed on an area of a first substrate and an array disposed on a second substrate. FIGS. 3A-3C depict a side view and a top view of an exemplary angled closure workflow 300 for sandwiching a first substrate (e.g., slide 303) having a biological sample 302 and a second substrate (e.g., slide 304 having capture probes 306) in accordance with some exemplary implementations.

FIG. 3A depicts the first substrate (e.g., slide 303 including a biological sample 302) angled over (superior to) the second substrate (e.g., slide 304). As shown, reagent medium (e.g., permeabilization solution) 305 is located on the spacer 310 toward the right-hand side of the side view in FIG. 3A. While FIG. 3A depicts the reagent medium on the right-hand side of side view, it should be understood that such depiction is not meant to be limiting as to the location of the reagent medium on the spacer.

FIG. 3B shows that as the first substrate lowers and/or as the second substrate rises, the dropped side of the first substrate (e.g., a side of the slide 303 angled toward the slide 304) may contact the reagent medium 305. The dropped side of the slide 303 may urge the reagent medium 305 toward the opposite direction (e.g., towards an opposite side of the spacer 310, towards an opposite side of the slide 303 relative to the dropped side). For example, in the side view of FIG. 3B the reagent medium 305 may be urged from right to left as the sandwich is formed.

In some embodiments, the first substrate and/or the second substrate are further moved to achieve an approximately parallel arrangement of the first substrate and the second substrate.

FIG. 3C depicts a full closure of the sandwich between the first substrate and the second substrate with the spacer 310 contacting both the first substrate and the second substrate and maintaining a separation distance and optionally the approximately parallel arrangement between the two substrates. As shown in the top view of FIG. 3C, the spacer 310 fully encloses and surrounds the biological sample 302 and the capture probes 306, and the spacer 310 form the sides of chamber 350 which holds a volume of the reagent medium 305.

While FIG. 3C depicts the first substrate (e.g., the slide 303 including biological sample 302) angled over (superior to) the second substrate (e.g., slide 304) and the second substrate comprising the spacer 310, it should be understood that an exemplary angled closure workflow can include the second substrate angled over (superior to) the first substrate and the first substrate comprising the spacer 310.

It may be desirable that the reagent medium be free from air bubbles between the substrates to facilitate transfer of target analytes with spatial information. Additionally, air bubbles present between the substrates may obscure at least a portion of an image capture of a desired region of interest. Accordingly, it may be desirable to ensure or encourage suppression and/or elimination of air bubbles between the two substrates (e.g., slide 303 and slide 304) during a permeabilization step (e.g., step 104). In some aspects, it may be possible to reduce or eliminate bubble formation between the substrates using a variety of filling methods and/or closing methods. In some instances, the first substrate and the second substrate are arranged in an angled sandwich assembly as described herein. For example, during the sandwiching of the two substrates (e.g., the slide 303 and the slide 304), an angled closure workflow may be used to suppress or eliminate bubble formation.

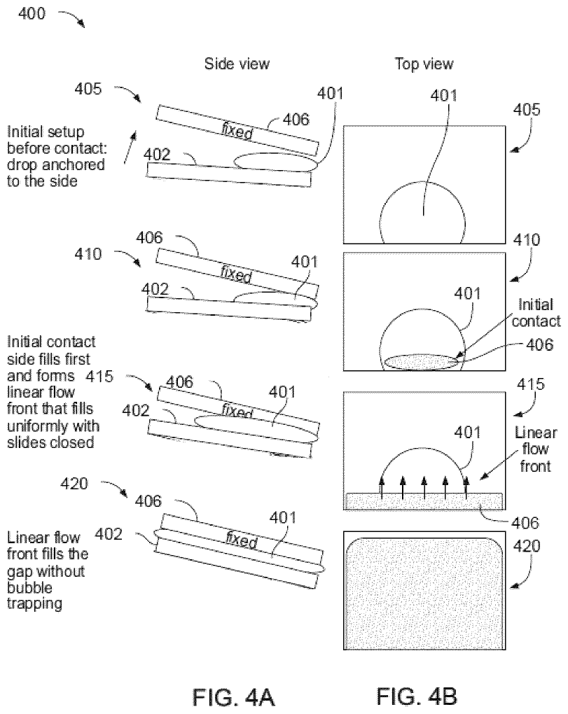

FIG. 4A is a side view of the angled closure workflow 400 in accordance with some exemplary implementations. FIG. 4B is a top view of the angled closure workflow 400 in accordance with some exemplary implementations. As shown at step 405, reagent medium 401 is positioned to the side of the substrate 402.

At step 410, the dropped side of the angled substrate 406 contacts the reagent medium 401 first. The contact of the substrate 406 with the reagent medium 401 may form a linear or low curvature flow front that fills the gap between the two substrates 406 and 402 uniformly with the slides closed.

At step 415, the substrate 406 is further lowered toward the substrate 402 (or the substrate 402 is raised up toward the substrate 406) and the dropped side of the substrate 406 may contact and urge the reagent medium toward the side opposite the dropped side, thereby creating a linear or low curvature flow front that may prevent or reduce bubble trapping between the substrates.

At step 420, the reagent medium 401 fills the gap between the substrate 406 and the substrate 402. The linear flow front of the liquid reagent may be formed by squeezing the reagent medium 401 volume along the contact side of the substrate 402 and/or the substrate 406. Additionally, capillary flow may also contribute to filling the gap area.

In some embodiments, the reagent medium (e.g., 105 in FIG. 1A) comprises a permeabilization agent. In some embodiments, following initial contact between the biological sample and a permeabilization agent, the permeabilization agent can be removed from contact with the biological sample (e.g., by opening the sample holder). Suitable agents for this purpose include, but are not limited to, organic solvents (e.g., acetone, ethanol, or methanol), cross-linking agents (e.g., paraformaldehyde), detergents (e.g., saponin, Triton X-100™, Tween-20™, SDS), and enzymes (e.g., trypsin or other proteases (e.g., proteinase K). In some embodiments, the detergent is an anionic detergent (e.g., SDS or N-lauroylsarcosine sodium salt solution).

In some embodiments, the reagent medium comprises a lysis reagent. Lysis solutions can include ionic surfactants such as, for example, sarkosyl and SDS. More generally, chemical lysis agents can include, without limitation, organic solvents, chelating agents, detergents, surfactants, and chaotropic agents. In some embodiments, the reagent medium comprises a protease. Exemplary proteases include, e.g., pepsin, trypsin, elastase, and proteinase K. In some embodiments, the reagent medium comprises a nuclease. In some embodiments, the nuclease comprises an RNase. In some embodiments, the RNase is selected from RNase A, RNase C, RNase H, and RNase I. In some embodiments, the reagent medium comprises one or more of SDS or a sodium salt thereof, proteinase K, pepsin, N-lauroylsarcosine, and RNase.

In some embodiments, the reagent medium comprises polyethylene glycol (PEG). In some embodiments, the PEG molecular weight is from about 2K to about 16K. In some embodiments, the PEG is about 2K, about 3K, about 4K, about 5K, about 6K, about 7K, about 8K, about 9K, about 10K, about 11K, about 12K, about 13K, about 14K, about 15K, or about 16K. In some embodiments, the PEG is present at a concentration from about 2% to about 25%, from about 4% to about 23%, from about 6% to about 21%, or from about 8% to about 20% (v/v).

In certain embodiments, a dried permeabilization reagent is applied or formed as a layer on the first substrate, the second substrate, or both prior to contacting the biological sample with the array. For example, a permeabilization reagent can be deposited in solution on the first substrate or the second substrate or both and then dried.

In some instances, the aligned portions of the biological sample and the array are in contact with the reagent medium for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the array are in contact with the reagent medium for about 1-60 minutes.

In some instances, the device is configured to control a temperature of the first and second substrates. In some embodiments, the temperature of the first and second members is lowered to a first temperature that is below room temperature.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of PCT Publication No. WO2020/176788 and/or U.S. Patent Application Publication No. 2020/0277663 regarding extended capture probes, which is herein incorporated by reference). In some cases, capture probes may be configured to form ligation products with a template (e.g., a DNA or RNA template, such as an analyte or an intermediate agent, or portion thereof), thereby creating ligation products that serve as proxies for the template.

As used herein, an “extended capture probe” refers to a capture probe having additional nucleotides added to a terminus (e.g., a 3′ or 5′ end) of the capture probe, thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using a reverse transcriptase. In some embodiments, the capture probe is extended using one or more DNA polymerases. In some embodiments, the extended capture probes include the sequence of the capture domain, the sequence of the spatial barcode of the capture probe, and the complementary sequence of the template used for extension of the capture probe.

In some embodiments, extended capture probes are amplified (e.g., in bulk solution or on the array) to yield quantities that are sufficient for downstream analysis, e.g., sequencing. In some embodiments, extended capture probes (e.g., DNA molecules) can act as templates for an amplification reaction (e.g., a polymerase chain reaction).

Additional variants of spatial analysis methods, including in some embodiments, an imaging step, are described in Section (II)(a) of PCT Publication No. WO2020/176788 and/or U.S. Patent Application Publication No. 2020/0277663, which is herein incorporated by reference. Analysis of captured analytes (and/or intermediate agents or portions thereof), for example, including sample removal, extension of capture probes using the captured analyte as a template, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of PCT Publication No. WO2020/176788 and/or U.S. Patent Application Publication No. 2020/0277663, which is herein incorporated by reference. Some quality control measures are described in Section (II)(h) of PCT Publication No. WO2020/176788 and/or U.S. Patent Application Publication No. 2020/0277663, which is herein incorporated by reference.

Spatial information can provide information of medical importance. For example, the methods described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication Nos. 2021/0140982, 2021/0198741, and 2021/0199660, each of which is herein incorporated by reference in its entirety.

Spatial information can provide information of biological importance. For example, the methods described herein can allow for: identification of transcriptome and/or proteome expression profiles (e.g., in healthy and/or diseased tissue); identification of multiple analyte types in close proximity (e.g., nearest neighbor or proximity based analysis); determination of up-regulated and/or down-regulated genes and/or proteins in diseased tissue; characterization of tumor microenvironments; characterization of tumor immune responses; characterization of cells types and their co-localization in healthy and diseased tissue; and identification of genetic variants within tissues (e.g., based on gene and/or protein expression profiles associated with specific disease or disorder biomarkers).

For spatial array-based methods, a substrate may function as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of PCT Publication No. WO2020/176788 and/or U.S. Patent Application Publication No. 2020/0277663, which is herein incorporated by reference. Exemplary features and geometric attributes of an array can be found in Sections (II)(d)(i), (II)(d)(iii), and (II)(d)(iv) of PCT Publication No. WO2020/176788 and/or U.S. Patent Application Publication No. 2020/0277663, which is herein incorporated by reference.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads or wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(c) of PCT Publication No. WO2020/176788 and/or U.S. Patent Application Publication No. 2020/0277663, which is herein incorporated by reference.

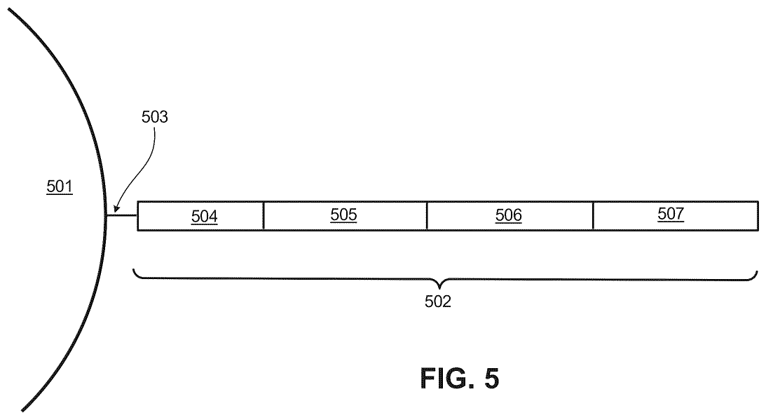

FIG. 5 is a schematic diagram showing an exemplary capture probe, as described herein. As shown, the capture probe 502 is optionally coupled to a feature 501 by a cleavage domain 503, such as a disulfide linker. The capture probe can include a functional sequence 504 that is useful for subsequent processing. The functional sequence 504 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 505. The capture probe can also include a unique molecular identifier (UMI) sequence 506. While FIG. 5 shows the spatial barcode 505 as being located upstream (5′) of UMI sequence 506, it is to be understood that capture probes wherein UMI sequence 506 is located upstream (5′) of the spatial barcode 505 is also suitable for use in any of the methods described herein. The capture probe can also include a capture domain 507 to facilitate capture of a target analyte. The capture domain can have a sequence complementary to a sequence of a nucleic acid analyte. The capture domain can have a sequence complementary to a connected probe described herein. The capture domain can have a sequence complementary to an analyte capture sequence present in an analyte capture agent. The capture domain can have a sequence complementary to a splint oligonucleotide. A splint oligonucleotide, in addition to having a sequence complementary to a capture domain of a capture probe, can have a sequence complementary to a sequence of a nucleic acid analyte, a portion of a connected probe described herein, a capture handle sequence described herein, and/or a methylated adaptor described herein.

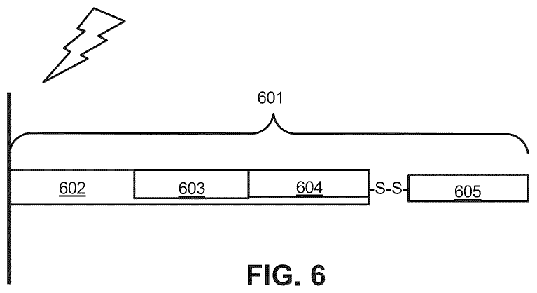

FIG. 6 is a schematic illustrating a cleavable capture probe, wherein the cleaved capture probe can enter into a non-permeabilized cell and bind to analytes within the cell. The capture probe 601 can contain a cleavage domain 602, a cell penetrating peptide 603, a reporter molecule 604, and a disulfide bond (—S—S—). 605 represents all other parts of a capture probe, for example, a spatial barcode and a capture domain.

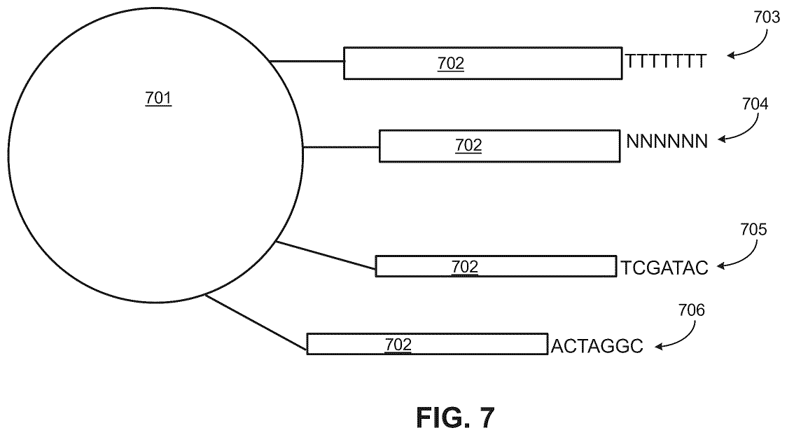

FIG. 7 is a schematic diagram of an exemplary multiplexed spatially-barcoded feature. In FIG. 7, the feature 701 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may include four different types of spatially-barcoded capture probes, each type of spatially-barcoded capture probe possessing the spatial barcode 702. Once type of capture probe associated with the feature can include the spatial barcode 702 in combination with a poly(T) capture domain 703, designed to capture mRNA target analytes. A second type of capture probe associated with the feature can include the spatial barcode 702 in combination with a random N-mer capture domain 704 for gDNA analysis. A third type of capture probe associated with the feature can include the spatial barcode 702 in combination with a capture domain complementary to the analyte capture agent of interest 705. A fourth type of capture probe associated with the feature can include the spatial barcode 702 in combination with a capture probe that can specifically bind a nucleic acid molecule 706 that can function in a CRISPR assay (e.g., CRISPR/Cas9). While only four different capture probe-barcoded constructs are shown in FIG. 7, capture-probe barcoded constructs can be tailored for analyses of any given analyte associated with a nucleic acid and capable of binding with such a construct. For example, the schemes shown in FIG. 7 can also be used for concurrent analysis of other analytes disclosed herein, including, but not limited to: (a) mRNA, a lineage tracing construct, cell surface or intracellular proteins and/or metabolites, and gDNA; (b) mRNA, accessible chromatin (e.g., ATAC-seq, DNase-seq, and/or MNase-seq), cell surface or intracellular proteins and/or metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V (D) J sequence of an immune cell receptor (e.g., T-cell receptor). In some embodiments, a perturbation agent can be a small molecule, an antibody, a drug, an aptamer, a miRNA, a physical environmental (e.g., temperature) change, or any other known perturbation agents.

The functional sequences can generally be selected for compatibility with any of a variety of different sequencing systems, e.g., ION TORRENT PROTON® (Thermo Fisher Scientific) or PGM® (Life Technologies), ILLUMINA® sequencing instruments (Illumina, Inc,), PACBIO® (Pacific Biosciences of California, Inc), OXFORD NANOPORE TECHNOLOGIES®, etc., and the requirements thereof. In some embodiments, functional sequences can be selected for compatibility with non-commercialized sequencing systems. Examples of such sequencing systems and techniques, for which suitable functional sequences can be used, include (but are not limited to) ION TORRENT PROTON® or PGM®, ILLUMINA® sequencing, PACBIOR SMRT sequencing, and), OXFORD NANOPORE TECHNOLOGIES® sequencing. Further, in some embodiments, functional sequences can be selected for compatibility with other sequencing systems, including non-commercialized sequencing systems.

In some embodiments, the spatial barcode 505 and functional sequence 504 are common to all of the probes attached to a given feature. In some embodiments, the UMI sequence 506 of a capture probe attached to a given feature is different from the UMI sequence of a different capture probe attached to the given feature.

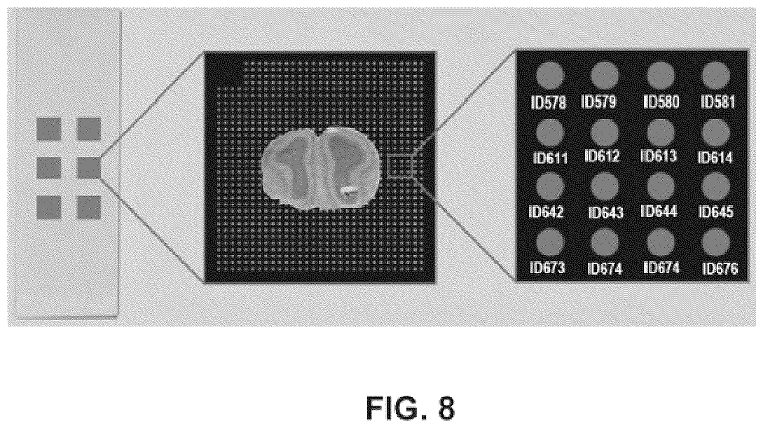

FIG. 8 depicts an exemplary arrangement of barcoded features within an array. From left to right, FIG. 8 shows (left) a slide including six spatially-barcoded arrays, (center) an enlarged schematic of one of the six spatially-barcoded arrays, showing a grid of barcoded features in relation to a biological sample, and (right) an enlarged schematic of one section of an array, showing the specific identification of multiple features within the array (e.g., labelled as ID578, ID579, ID580, etc.).

In some embodiments, more than one analyte type (e.g., nucleic acids and proteins) from a biological sample can be detected (e.g., simultaneously or sequentially) using any appropriate multiplexing technique, such as those described in Section (IV) of PCT Publication No. WO2020/176788 and/or U.S. Patent Application Publication No. 2020/0277663, which is herein incorporated by reference.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of PCT Publication No. WO2020/176788 and/or U.S. Patent Application Publication No. 2020/0277663, which is herein incorporated by reference.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128, which is herein incorporated by reference in its entirety. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture probe binding domain (e.g., a poly(A) sequence or a non-homopolymeric sequence). After hybridization to the analyte, a ligase (e.g., a T4 RNA ligase (Rn12), a PBCV-1 DNA Ligase or Chlorella virus DNA Ligase, a single-stranded DNA ligase, or a T4 DNA ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNase H). In some instances, the ligation product is removed using heat. In some instances, the ligation product is removed using KOH. The released ligation product can then be captured by capture probes (e.g., instead of direct capture of an analyte) on an array, optionally amplified, and sequenced, thus determining the location, and optionally, the abundance of the analyte in the biological sample.

In some instances, one or both of the oligonucleotides may hybridize to genomic DNA (gDNA), which can lead to false positive sequencing data from ligation events on gDNA (off target) in addition to the desired (on target) ligation events on target nucleic acids (e.g., mRNA). Thus, in some embodiments, the disclosed methods can include contacting the biological sample with a deoxyribonuclease (DNase). The DNase can be an endonuclease or exonuclease. In some embodiments, the DNase digests single-stranded and/or double-stranded DNA. Suitable DNases include, without limitation, a DNase I and a DNase II. Use of a DNase as described can mitigate false positive sequencing data from off target gDNA ligation events.

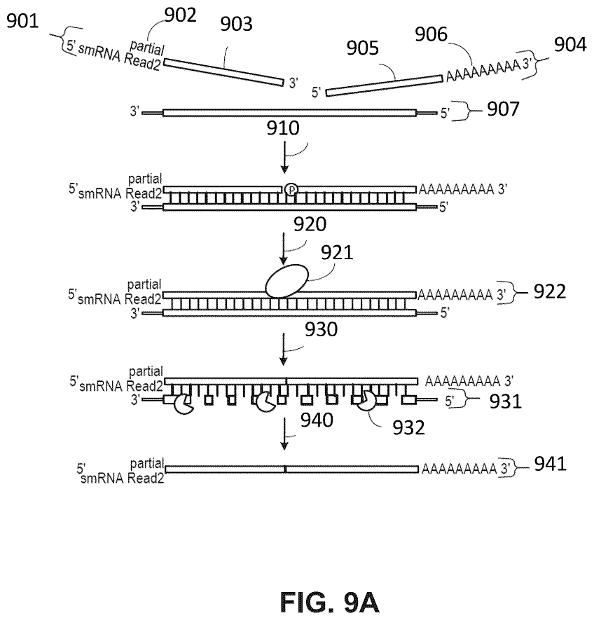

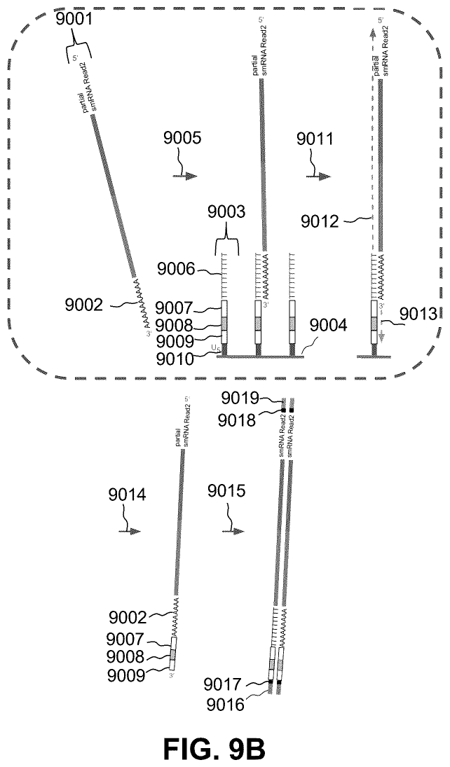

A non-limiting example of templated ligation methods disclosed herein is depicted in FIG. 9A. After a biological sample is contacted with a substrate including a plurality of capture probes and contacted with (a) a first probe 901 having a target-hybridization sequence 903 and a primer sequence 902 and (b) a second probe 904 having a target-hybridization sequence 905 and a capture domain (e.g., a poly(A) sequence) 906, the first probe 901 and the second probe 904 hybridize 910 to an analyte 907. A ligase 921 ligates 920 the first probe 901 to the second probe 904, thereby generating a ligation product 922. The ligation product 922 is then released 930 from the analyte 931 by digesting the analyte 907 using an endoribonuclease 932. The sample is permeabilized 940 and the ligation product 941 is able to hybridize to a capture probe on the substrate. Methods and compositions for spatial detection using templated ligation have been described in PCT Publication. No. WO 2021/133849 A1, U.S. Pat. Nos. 11,332,790 and 11,505,828, each of which is incorporated by reference in its entirety.

In some embodiments, as shown in FIG. 9B, the ligation product 9001 includes a capture probe capture domain 9002, which can bind to a capture probe 9003 (e.g., a capture probe immobilized, directly or indirectly, on a substrate 9004). In some embodiments, methods provided herein include contacting 9005 a biological sample with a substrate 9004, wherein the capture probe 9003 is affixed to the substrate (e.g., immobilized to the substrate, directly or indirectly). In some embodiments, the capture probe capture domain 9002 of the ligated product 9001 specifically binds to the capture domain 9006. The capture probe can also include a unique molecular identifier (UMI) 9007, a spatial barcode 9008, a functional sequence 9009, and a cleavage domain 9010.

In some embodiments, methods provided herein include permeabilization of the biological sample such that the capture probe can more easily capture the ligation products (i.e., compared to no permeabilization). In some embodiments, polymerization (e.g., reverse transcription (RT)) reagents can be added to permeabilized biological samples. Incubation with the polymerization reagents can be used to extend the capture probes 9011 to produce spatially-barcoded full-length cDNA 9012 and 9013 from the captured ligation products (e.g., ligation products). The ligation products can be extended using the capture probe as a template to include a complement of the capture probe, thereby generating extended ligation products.

In some embodiments, the extended ligation products can be denatured 9014, released from the capture probe, and transferred (e.g., to a clean tube) for amplification and/or library construction. The spatially-barcoded ligation products can be amplified 9015 via PCR prior to library construction. P5 9016, i5 9017, i7 9018, and P7 9019 sequences can be used as sample indexes. The amplicons can then be sequenced using paired-end sequencing using TruSeq Read 1 and TruSeq Read 2 as sequencing primer sites.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b) (ix) of PCT Publication No. WO2020/176788 and/or Section (II)(b)(viii) U.S. Patent Application Publication No. 2020/0277663, which is herein incorporated by reference.

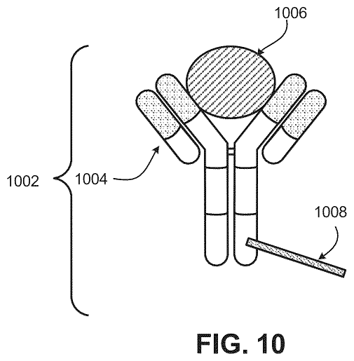

FIG. 10 is a schematic diagram of an exemplary analyte capture agent 1002 comprised of an analyte binding moiety 1004 and an analyte-binding moiety barcode domain 1008. The exemplary analyte binding moiety 1004 is capable of binding to an analyte 1006 and the analyte capture agent 1002 is capable of interacting with a spatially-barcoded capture probe. The analyte binding moiety 1004 can bind to the analyte 1006 with high affinity and/or with high specificity. The analyte capture agent 1002 can include: (i) an analyte binding moiety barcode domain 1008, which serves to identify the analyte binding moiety, and (ii) an analyte capture sequence, which can hybridize to at least a portion or an entirety of a capture domain of a capture probe. The analyte binding moiety 1004 can include a polypeptide and/or an aptamer. The analyte binding moiety 1004 can include an antibody or antibody fragment (e.g., an antigen-binding fragment).

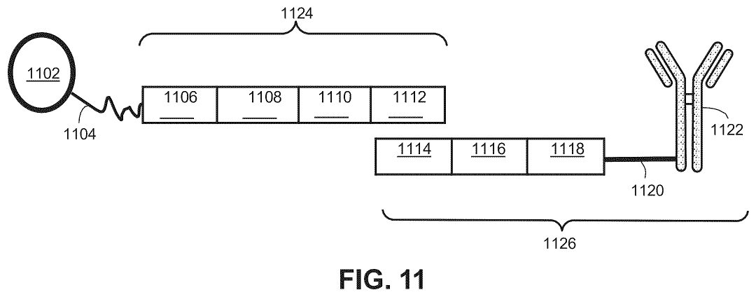

FIG. 11 is a schematic diagram depicting an exemplary interaction between a feature-immobilized capture probe 1124 and an analyte capture agent 1126. The feature-immobilized capture probe 1124 can include a spatial barcode 1108 as well as functional sequence 1106 and a UMI 1110, as described elsewhere herein. The capture probe can be affixed 1104 to a feature such as a bead 1102. The capture probe 1124 can also include a capture domain 1112 that is capable of binding to an analyte capture agent 1126. The analyte binding moiety barcode domain of the analyte capture agent 1126 can include functional sequence 1118, analyte binding moiety barcode 1116, and an analyte capture sequence 1114 that is capable of binding (e.g., hybridizing) to the capture domain 1112 of the capture probe 1124. The analyte capture agent 1126 can also include a linker 1120 that allows the analyte binding moiety barcode domain (e.g., including the functional sequence 1118, analyte binding moiety barcode 1116, and analyte capture sequence 1114) to couple to the analyte binding moiety 1122. In some embodiments, the linker 1120 is a cleavable linker. In some embodiments, the cleavable linker is a photo-cleavable linker, a UV-cleavable linker, chemical-cleavable, thermal-cleavable, or an enzyme cleavable linker. In some instances, the cleavable linker is a disulfide linker. A disulfide linker can be cleaved by use of a reducing agent, such as dithiothreitol (DTT), beta-mercaptoethanol (BME), or Tris (2-carboxyethyl) phosphine (TCEP).

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the captured analytes are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that each spatial barcode is uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location or a fiducial marker) of the array. Accordingly, each feature location has an “address” or location in the coordinate space of the array.

Some exemplary spatial analysis workflows are described in the Exemplary Embodiments section of PCT Publication No. WO2020/176788 and/or U.S. Patent Application Publication No. 2020/0277663, which is herein incorporated by reference. See, for example, the Exemplary embodiment starting with “In some non-limiting examples of the workflows described herein, the sample can be immersed . . . ” of PCT Publication No. WO2020/176788 and/or U.S. Patent Application Publication No. 2020/0277663, which is herein incorporated by reference. See also, e.g., the Visium Spatial Gene Expression Reagent Kits User Guide (e.g., Rev F, dated January 2022) and/or the Visium Spatial Gene Expression Reagent Kits-Tissue Optimization User Guide (e.g., Rev E, dated February 2022), each of which is herein incorporated by reference in its entirety.

In some embodiments, spatial analysis can be performed using dedicated hardware and/or software, such as any of the systems described in Sections (II)(c) (ii) and/or (V) of PCT Publication No. WO2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of PCT Publication No. WO2020/123320, which is herein incorporated by reference.

Suitable systems for performing spatial analysis can include components such as a chamber (e.g., a flow cell or a scalable, fluid-tight chamber) for containing a biological sample. The biological sample can be mounted, for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable, and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Publication No. WO2021/102003 and/or U.S. Patent Application Publication No. 2021/0150707, each of which is incorporated herein by reference in its entirety.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two-dimensional and/or three-dimensional map of the analyte presence and/or level are described in PCT Publication No. WO2020/053655 and spatial analysis methods are generally described in PCT Publication No. WO2021/102039 and/or U.S. Patent Application Publication No. 2021/0155982, each of which is incorporated herein by reference in its entirety.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of PCT Publication Nos. WO2020/123320, WO 2021/102005, and/or U.S. Patent Application Publication No. 2021/0158522, each of which is incorporated herein by reference in its entirety. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### II. Methods of Strand-Displacement Amplification of Target Nucleic Acids and Compositions for Use Therein

Methods for amplification of a target nucleic acid (e.g., a captured nucleic acid analyte from a biological sample) containing single-stranded DNA (ssDNA), have been developed. Compositions for use in the disclosed method are also expressly described herein.

In some forms, a target nucleic acid is amplified to yield quantities that are sufficient for analysis, e.g., via DNA sequencing. In some forms, a first strand of a target nucleic acid (e.g., DNA and/or cDNA molecules) acts as a template for the amplification reaction (e.g., an RNase mediated strand displacement reaction).

In some forms, amplifying a target nucleic acid can function, e.g., to release copies of the target nucleic acid (amplification products or amplicons) from the surface of a substrate.

In some forms, an amplified target nucleic acid or complement thereof is released, while the target nucleic acid itself remains attached to a substrate.

In some embodiments, the methods provide a complementary (replicate) nucleic acid from a single-stranded target nucleic acid. In some forms, the single-stranded target nucleic acid is attached to a substrate. An exemplary target nucleic acid is an extended captured probe, such as an extended capture probe conjugated to a substrate e.g., that is part of an array. The methods employ strand displacing DNA polymerases to provide one or more copies of a nucleic acid having a nucleotide sequence that is the same or complementary to a target nucleic acid. Typically, the methods increase the amount of nucleic acids having a desired nucleotide sequence, or having the complement of a desired nucleotide sequence. In some forms, the methods increase the amount of a nucleic acid having the sequence of, or complementary to, a target nucleic acid by at least 100%, up to 1000%, or more than 1000%, such as up to 10,000% or up to 100,000% the original amount of the target nucleic acid prior to the methods. In some forms, the methods increase the amount of target nucleic acids in a mixture of target and non-target nucleic acids. Therefore, in some forms, the methods increase the ratio of target to non-target nucleic acids within a mixture of different nucleic acid species.

The target nucleic acid that is to be amplified according to the methods can be a single stranded (ss) nucleic acid, or can be one strand of a double stranded (ds) nucleic acid. In an exemplary form, the methods provide a multiplicity of copies of a single stranded nucleic acid having a nucleotide sequence that corresponds to the nucleotide sequence of a target nucleic acid immobilized on a solid support, such as an array. In some forms, the methods amplify a target nucleic acid at a constant temperature and/or upon addition of a single reaction mixture to a sample. In some forms, the methods provide one or more copies of a single stranded nucleic acid having a nucleotide sequence complementary to the nucleotide sequence of a target nucleic acid. In other forms, the methods provide one or more copies of a single stranded nucleic acid having a nucleotide sequence of a target nucleic acid. In some forms, the methods are carried out subsequent to one or more of the described methods for spatial analysis. For example, in some embodiments, the spatial analysis is performed using capture probes as described in Section I.

The described methods for increasing the amount of a target nucleic acid within a sample are designed to be implemented within any spatial analysis protocol, for example, to increase the number of nucleic acids having a sequence corresponding to a captured analyte. Therefore, methods including amplification of a target nucleic acid including the nucleotide sequence of all or part of an analyte or proxy thereof (e.g., intermediate agent, ligation product) captured and arrayed as part of a spatial analysis workflow are also provided. In some forms, one or more of the described methods for amplifying a target nucleic acid are implemented within one or more steps of a spatial analysis protocol. Typically, one or more steps for amplifying a target nucleic acid are performed subsequent to capturing an analyte from a sample that is subject to a spatial analysis assay. In some forms, the methods enhance the resolution, sensitivity, specificity and/or accuracy of spatial analysis of a target nucleic acid analyte within a biological sample by increasing the amount of a target nucleic acid available for analysis. For example, in some forms, the methods amplify a target nucleic acid that includes a sequence of, or complementary to, a target analyte from a sample, such as a biological sample, to enhance the accuracy and/or efficacy of determination of the sequence of the target analyte. In some forms, a method disclosed herein relates to the detection of target nucleic acid sequences (e.g., target RNAs) in situ at one or more locations in a blocked sample wherein background signal is reduced and/or sensitivity of the spatial analysis is increased by the selective amplification of target nucleic acids according to the described methods. In some aspects, the amplification of target nucleic acids according to the described methods results in improved sensitivity (number of detected signals), specificity, signal intensity, and/or improved signal to noise, compared to an equivalent spatial analysis of an equivalent sample wherein target-specific amplification according to the described methods has not been performed.

The methods typically include steps of removing, hybridizing and extending a selectively removable or digestible primer with a strand-displacing DNA polymerase once or more than once to provide a multiplicity of copies of a target DNA molecule. Any of the described methods can further including repeating each of the steps of removing, hybridizing and extending the second primer with a strand-displacing DNA polymerase once or more than once to provide a multiplicity of copies of the target DNA molecule. In some forms, each of the steps of removing, hybridizing and extending the second primer with a strand-displacing DNA polymerase is carried out at a constant temperature.

### A. Strand-Displacement Amplification of Single-Stranded Target DNA

Methods for strand-displacement amplification of target nucleic acids, e.g., a target DNA molecule, are described. Methods for strand-displacement amplification of a single-stranded target DNA molecule are also provided. In an exemplary form, a method for amplification of a single-stranded (ss) target DNA molecule includes one or more steps of:
(a) hybridizing to the target DNA molecule a first primer including both a first region, including DNA substantially complementary to the target DNA molecule, and a second region including RNA nucleotides (or DNA nucleotides including a unique target sequence, or inosine nucleotides);
(b) extending the first primer from its 3′ end using the target DNA molecule as a template and extending 3′ end of the target DNA molecule using the first primer as a template, to form an extended hybrid double-stranded (ds) DNA molecule;
(c) removing the second region from the extended hybrid dsDNA molecule to form a truncated dsDNA molecule including a region of ssDNA;
(d) hybridizing a second primer to the region of ssDNA of the truncated dsDNA molecule to form a hybridized second molecule including the second region including one or more of RNA nucleotides (or DNA nucleotides including a unique target sequence, or inosine nucleotides); and
(c) extending the hybridized second primer of the hybridized second molecule using a strand-displacing DNA polymerase to provide an extended hybrid double-stranded (ds) DNA molecule and a displaced single-stranded (ss) DNA molecule that is complementary to the sequence of the target nucleic acid; and
(f) optionally repeating steps (c), (d) and (c) to provide a multiplicity of copies of a displaced single-stranded (ss) DNA molecule that is complementary to the sequence of the target nucleic acid.

In some forms, the second region of the first primer exclusively includes RNA nucleotides. In some forms, about 75%, about 80%, about 85%, about 90%, about 95%, about 96%, about 97%, about 98%, about 99% or about 100% of the nucleotides of the second region of the first primer includes RNA nucleotides.

A target nucleic acid can be any suitable nucleic acid, e.g., preferably DNA (e.g., cDNA, gDNA). The target nucleic acid can be double stranded or single stranded. In some forms, a target nucleic acid includes a spatial barcode, a capture domain, and one or more one or more functional domains (e.g., a sequencing primer binding site, an amplification domain). In some forms, a target nucleic acid includes a spatial barcode, a unique molecular identifier, a capture domain, and one or more one or more functional domains (e.g., a sequencing primer binding site, an amplification domain). In some forms, a target nucleic acid includes a spatial barcode, a unique molecular identifier, a capture domain, a sequence corresponding to a target analyte (e.g., cDNA, ligated RTL probes, an intermediate agent), and one or more functional domains (e.g., a sequencing primer binding site, an amplification domain).

In some forms, two or more of steps (a), (b), (c), (d), and/or (c), are carried out following a single active step, for example, where a single reaction mixture including reagents necessary for two or more of steps (a), (b), (c), (d), and/or (e) is used. In an exemplary form, a single reaction mixture including a first primer (e.g., oligonucleotide primer), a second primer (e.g., oligonucleotide primer), a strand displacing polymerase and an RNase enzyme is used, such that each of steps (a), (b), (c), (d), and/or (c), are carried out following a single active step of contacting the target nucleic acid with the reaction mixture under suitable conditions for a strand displacement reaction (e.g., an isothermal strand displacement) reaction to occur.

Each of these steps is described in greater detail, below.

### (a) Hybridizing a First Primer to the Target DNA

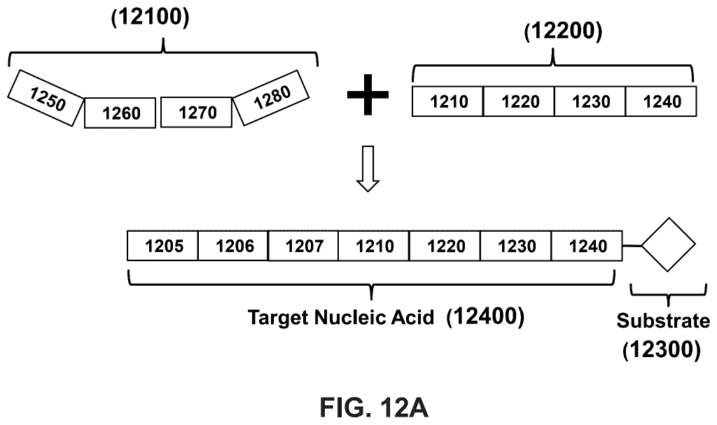

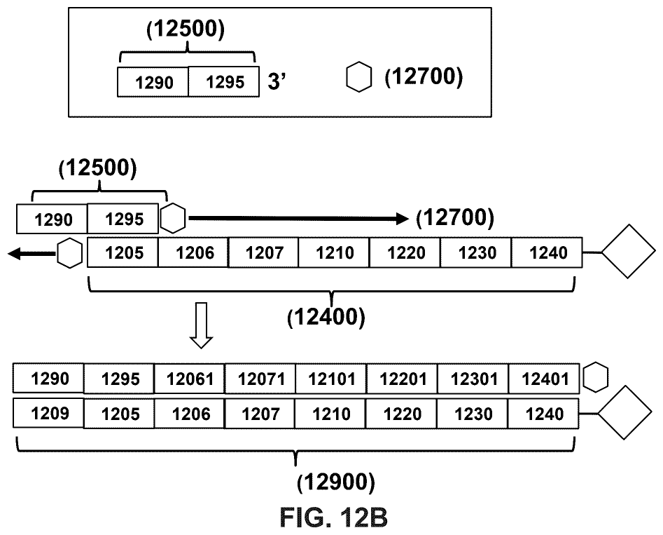

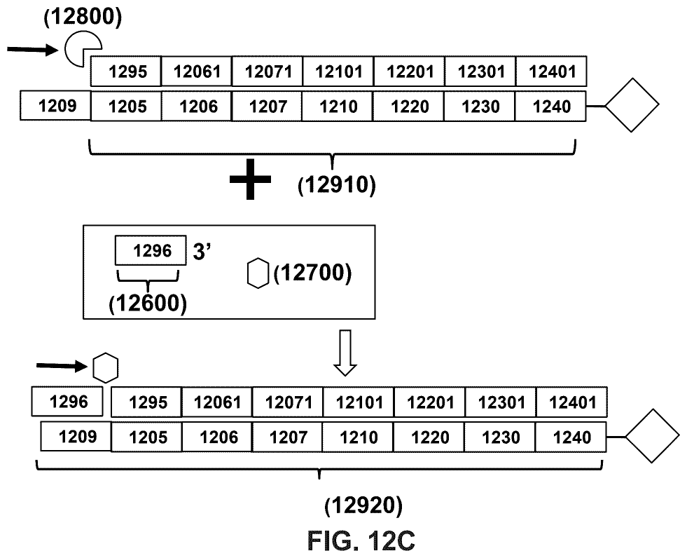

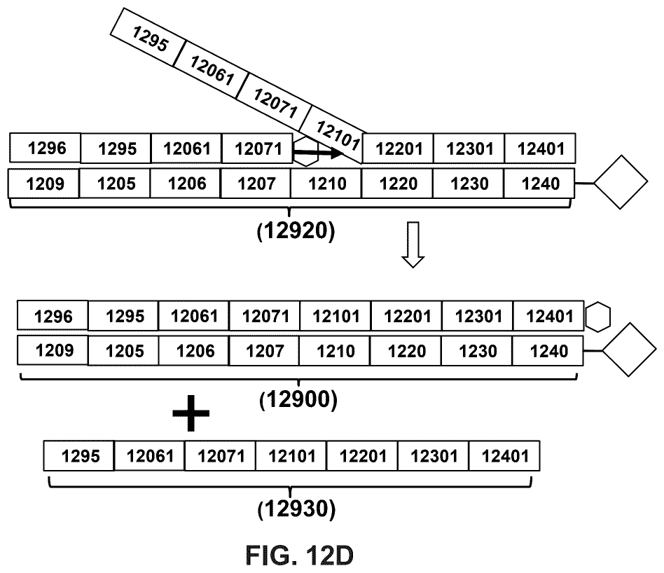

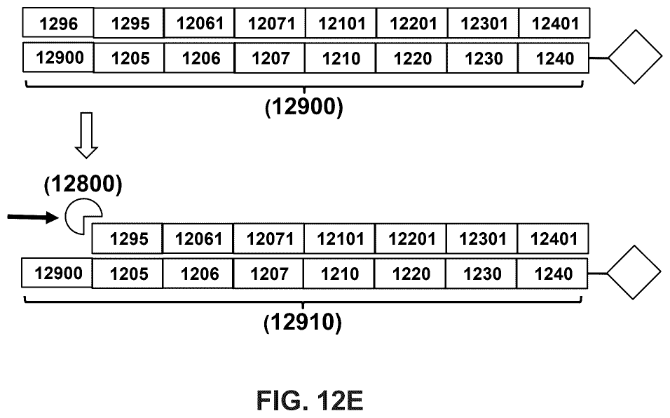

The methods require a step of hybridizing a first primer (e.g., oligonucleotide primer) to the target nucleic acid, to provide a partially double stranded intermediate molecule including the primer and the target nucleic acid (see, e.g., 12400 in FIG. 12B).

The methods hybridize a primer including a selectively removable domain to provide an amplified second strand complementary to the target DNA molecule including the removable domain at the 5′ end, such that removal of the removable domain provides a partially double-stranded DNA molecule having a 5′ “sticky end” that serves as a priming site for a second primer, which in turn includes a selectively removable nucleic acid domain and which is extended by a strand-displacing polymerase.

Optimal conditions for hybridizing a first primer to a target nucleic acid can be determined by one skilled in the art according to the size, composition and quantity of the target nucleic acid and the first primer. Typically, hybridization is carried out under conditions that do not denature or otherwise impact the conformation of the target nucleic acid.

### 1. First Oligonucleotide Primer Design

First oligonucleotide primers for use in the described methods are provided. As used herein, the terms “first primer” and “first oligonucleotide primer” are used interchangeably.

The first primer typically includes at least two functional nucleic acid “domains”, including a first “hybridization” domain and a second “hybrid” (removable) domain.

The first primer typically includes from about 8 to about 100 nucleotides, inclusive, or any integer (or range of integers) of nucleotides in between the indicated values, for example, between about 16 to about 64 nucleotides, inclusive, or between about 12 to about 24 nucleotides, inclusive. In some forms, the first primer includes about 16 nucleotides. In some embodiments, each of the functional nucleic acid “domains”, includes about 4, 5, 6, 7, 8, 9, 10, 11, 12, 13, 14, 15 or 16, or more nucleotides.

In some forms, the first primer includes more than a single type of nucleotide. For example, in some forms, the first primer includes both DNA and RNA, or DNA and inosine, or DNA and methylated DNA. In other forms, the first primer includes only DNA nucleotides, for example, where a first region of DNA includes a nucleotide sequence that functions to hybridize to the target nucleic acid, and where a second hybrid region includes a DNA nucleotide sequence that functions as an enzyme recognition sequence, such as a restriction endonuclease recognition sequence. Typically, the first primer includes in 5′ to 3′ orientation, the second hybrid region and the first region. When the hybridization domain is located at the 3′ region of the first primer, the second hybrid domain forms a 5′ overhang when the first primer hybridizes to the target nucleic acid.

In an exemplary form, the size of the first primer is between about 8 and about 100 nucleotides, inclusive, whereby the first region includes between about four and about thirty DNA nucleotides, inclusive, and the second hybrid region includes between about four and about thirty RNA nucleotides, inclusive.

a. First (Hybridization) Domain

The first oligonucleotide primer includes at least one first “hybridization” domain including a nucleotide sequence that is substantially complementary to a nucleotide sequence within the free 3′ end of a target nucleic acid. The first domain of the first primer is typically formed entirely or partially from DNA and includes a sufficient number of nucleotides to impart specificity for hybridizing to the target nucleic acid. In some forms, the sequence that is to be amplified is targeted by the first domain of the first primer. In some embodiments, the first domain includes about 4, 5, 6, 7, 8, 9, 10, 11, 12, 13, 14, 15 or 16, or more than 16 contiguous nucleotides. In an exemplary form, the first domain includes 8 nucleotides. The design of the first domain should include determination of a suitably unique sequence within the 3′ end of the target nucleic acid that is of sufficient length to ensure selective hybridization.

Generally, the first hybridization region is or includes any sequence that hybridizes to the 3′ end of the target nucleic acid, as required by the described methods. Generally, the target nucleic acid molecule can be any desirable target. In some forms, e.g., where the methods are implemented within one or more spatial analysis methods, the target nucleic acid molecule is the extended capture probe on the array. For example, in some forms, when the target nucleic acid molecule is a capture probe extended using RNA Templated Ligation (RTL), the first region of the first primer is the same sequence as a first priming site present within a left-hand RTL probe. In an exemplary form, 3′ end of a target nucleic acid includes a priming site for a first sequencing primer, such as a Read 2S domain. In an exemplary form, where the target nucleic acid includes an extended hybridized capture probe (1204), as indicated in FIG. 12A, 3′ end of the target nucleic acid includes a sequence that is complementary to all or a part of a sequencing primer sequence (Read 2S) region of the Left-hand portion of a RTL probe. Therefore, in an exemplary form, the first hybridization domain of the first primer includes a nucleotide sequence of a sequencing primer attachment sequence/binding site. Typically, the first domain is located at the 3′ region of the first primer.

b. Second Hybrid (Removable) Domain

The first oligonucleotide primer includes a second “hybrid” or “removable” domain/region. In some forms, the second hybrid region includes a removable or digestible nucleotide sequence that is or includes a substrate for an enzyme or reaction that digests and/or cleaves all or part of the domain when present in a double-stranded nucleic acid. In some forms, the second hybrid domain of the first primer is formed entirely from RNA and includes a sufficient number of RNA nucleotides that, when removed, provide a region of single-stranded nucleic acid overhang at 3′ end of a ds target nucleic acid, wherein the single-stranded overhang at 3′ end is sufficient for selective hybridization of a second oligonucleotide primer to the single-stranded overhang. In some forms, the second hybrid domain includes about 4, 5, 6, 7, 8, 9, 10, 11, 12, 13, 14, 15 or 16, or more than 16 contiguous nucleotides. In an exemplary form, the second hybrid domain includes 8 nucleotides. The design of the second hybrid domain should include determination of a suitably large sequence to ensure effective removal of the domain by a corresponding enzyme or reagent. Typically, the second hybrid domain is located at 5′ region of the first primer.

### 2. Exemplary First Oligonucleotide Primers

Compositions of first oligonucleotide primers and use thereof according to the described methods are provided.

Exemplary first oligonucleotide primers typically include two functional nucleic acid regions, or “domains”, including a first “hybridization” domain and a second “hybrid” (removable) domain. The terms “region” and “domain” are used interchangeably herein to refer to a functional or structural sub-component of a larger nucleic acid. Thus, the term “first domain” of the first primer as used herein is used interchangeably with the term “first region” of the first primer, and the term “second hybrid domain” of the first primer is used interchangeably with the terms “hybrid domain”, “hybrid region”, “second region”, “removable region”, “removable domain”, “second hybrid region”, “second removable region” or “second hybrid region” of the first primer.

Exemplary first oligonucleotide primers include, in 5′ to 3′ orientation, the second hybrid region and the first region. When the first hybridization domain is located at the 3′ region of the first primer, the second hybrid domain initially forms a 5′ overhang when the first primer hybridizes to the target nucleic acid. The second hybrid domain includes a removable nucleotide sequence that is or includes a substrate for an enzyme or reaction that digests and/or cleaves all or part of the second hybrid domain from within a double-stranded nucleic acid. An exemplary second hybrid domain is formed entirely from RNA and includes a sufficient number of RNA nucleotides that can be removed or digested to provide a region of single-stranded nucleic acids sufficient for selective hybridization of a second oligonucleotide primer to the single-stranded overhang. In some forms, the second hybrid domain includes about 4, 5, 6, 7, 8, 9, 10, 11, 12, 13, 14, 15 or 16, or more than 16 contiguous nucleotides. In an exemplary form, the second hybrid domain includes 8 nucleotides. The design of the second hybrid domain should include determination of a suitably large sequence to ensure effective removal of the domain by a corresponding enzyme or reagent. Typically, the second hybrid domain is located at 5′ region of the first primer.

In an exemplary form, the first oligonucleotide primer includes from about 8 to about 100 nucleotides, inclusive, whereby the first region includes between about four and about thirty DNA nucleotides, inclusive, and the second hybrid region includes between about 4 and about 30 RNA nucleotides, inclusive.

(b) Extending the first primer

Following hybridization of the first oligonucleotide primer in step (a), the methods include one or more steps of polymerase-based extension of the primer, using the target nucleic acid as a template, to provide a complementary second nucleic acid strand having a nucleotide sequence complementary to that of the target nucleic acid.

Typically, the extension includes use of a polymerase enzyme that is capable of strand displacement (strand displacing polymerase; SDP enzyme). Suitable SDP enzymes for use in the methods include SDP enzymes isolated or derived from Bacillus stearothermophilus such as, Bst2.0 or Bst3.0. The extension initiated by 3′ first hybridization domain/region of the first primer typically yields a second strand that includes the entire first oligonucleotide primer. The extension can also fill in 3′ gap formed by 5′ sticky ended overhang that is created by the hybrid domain of the first primer, to form an extended hybrid double-stranded (ds) DNA molecule (see, e.g., 12900 in FIG. 12B). For example, the SDP enzyme can extend the target nucleic acid using the first oligonucleotide primer as a template, thereby generating an extended hybrid double-stranded (ds) DNA molecule (see, e.g., 12900 in FIG. 12B). This can occur separately from or concurrently with the extension of the first primer using the target nucleic acid as a template.

Optimal conditions for extension of a first primer using a polymerase, such as an SDP, can be determined by one skilled in the art according to the size, composition and quantity of the target nucleic acid and the first primer. Typically, the extension is carried out under conditions that do not denature or otherwise impact the conformation of the target nucleic acid.

### (c) Removal of the Hybrid Domain

Following extension of the first oligonucleotide primer in step (b), the methods include one or more steps to selectively remove the hybrid domain of the extended hybrid double-stranded (ds) DNA molecule.

Typically, the methods remove the second hybrid region from the second strand amplified in step (b) by contacting the extended hybrid double-stranded (ds) DNA molecule with a suitable reagent to selectively remove the second hybrid region. For example, if the second hybrid region of the first oligonucleotide primer includes RNA, the step of removing the second hybrid region includes contacting the target nucleic acid with an endoribonuclease, e.g., an RNase enzyme, such as an RNase H. In some forms, the RNase H is RNase H1 or RNase H2. An exemplary RNase, (e.g., RNaseH) (12800)-mediated removal of the hybrid region (1290) of an extended hybrid double-stranded (ds) DNA molecule ((12900) of FIG. 12B) is depicted in FIG. 12C.

As depicted in FIG. 12C, the successful removal of the hybrid region provides a partially single stranded, truncated dsDNA molecule (12910) that includes a region of ssDNA at the 5′ end of the second (top) strand.

Optimal conditions for removal of a second hybrid region of a first oligonucleotide primer, such as RNA, using a suitable reagent, such as RNaseH, can be determined by one skilled in the art according to the size, composition and quantity of the hybrid region. Typically, the removal is carried out under conditions that do not denature or otherwise impact the conformation of the target nucleic acid.

### (d) Hybridizing a Second Primer to the Target DNA

Following removal of the hybrid region of the extended hybrid double-stranded (ds) DNA molecule in step (c), the methods include one or more steps to hybridize a second oligonucleotide primer to the region of ss nucleic acid of the truncated ds nucleic acid molecule. The second oligonucleotide primer typically is or includes all or part of the second hybrid region of the first oligonucleotide primer e.g., including removable components. In a preferred form, the second primer includes only RNA nucleotides.

Typically, the methods hybridize a second primer, formed entirely of selectively removable nucleotides, to the region of ss nucleic acid of the truncated ds nucleic acid molecule to re-constitute a ds nucleic acid that is amenable to amplification by a strand-displacing polymerase. The second primer hybridizes to the region of ssDNA of the truncated dsDNA molecule. In some forms, this hybridization leaves a nick (i.e., a single-stranded break) at 3′ end of the primer (see, e.g., 12910 in FIG. 12C).

Optimal conditions for hybridizing a second primer to a target nucleic acid can be determined by one skilled in the art according to the size, composition and quantity of the target nucleic acid and the second primer. Typically, hybridization is carried out under conditions that do not denature or otherwise impact the conformation of the target nucleic acid.

### 1. Exemplary Second Oligonucleotide Primers

Compositions of second oligonucleotide primers and uses thereof according to the described methods are also described.

Exemplary second oligonucleotide primers typically include a single removable (“hybrid”) nucleic acid region, or “domain”. The hybrid domain of the second oligonucleotide primer molecule typically includes a removable nucleotide sequence that is or includes a substrate for an enzyme or reaction that digests and/or cleaves all or part of the domain from within a double-stranded nucleic acid.

In some forms, an exemplary second oligonucleotide primer is formed entirely from RNA and includes a sufficient number of RNA nucleotides that, when removed, provide a region of single-stranded (“ss”) nucleic acid sufficient for selective hybridization of a further second oligonucleotide primer to the same single-stranded nucleic acid region.

In some forms, an exemplary second oligonucleotide primer molecule is formed entirely from DNA and inosine nucleotides and includes a sufficient number of inosine nucleotides that, when removed/digested, provide a region of ss nucleic acid sufficient for selective hybridization of a further second oligonucleotide primer to the same single-stranded nucleic acid region.

In some forms, an exemplary second oligonucleotide primer molecule is formed entirely from DNA and includes a sufficient number and sequence of DNA nucleotides that provide a unique recognition site for a restriction endonuclease that, when cleaved, provide a region of ss nucleic acid sufficient for selective hybridization of a further second oligonucleotide primer to the same single-stranded nucleic acid region.

In some forms, an exemplary second oligonucleotide primer is formed entirely from or includes methylated GATC nucleotides that provide a unique recognition site for a methylation-specific enzyme that, upon cleavage of the recognition site, provide a region of ss nucleic acid sufficient for selective hybridization of a further second oligonucleotide primer to the same single-stranded nucleic acid region.

In some forms, the second oligonucleotide primer includes about 4, 5, 6, 7, 8, 9, 10, 11, 12, 13, 14, 15 or 16, or more than 16 contiguous nucleotides. In an exemplary form, the hybrid domain includes 8 nucleotides. The design of the hybrid domain should include determination of a suitably large sequence to ensure effective removal of the domain by a corresponding enzyme or reagent. Typically, the hybrid domain is located at 5′ region of the first primer.

### (e) Extending the Hybridized Second Primer

Following hybridization of a second oligonucleotide primer to the region of ssDNA of the truncated dsDNA molecule in step (d), the methods include one or more steps to extend the hybridized second primer using a strand-displacing DNA polymerase enzyme, to provide an extended hybrid double-stranded (ds) DNA molecule and a displaced single-stranded (ss) DNA molecule that is complementary to the sequence of the target nucleic acid.

The extension reaction in step (e) preferably uses a strand displacing polymerase; SDP enzyme. In some forms, the extension is initiated from 3′ end of the second primer to yield a new (i.e., “third”) nucleic acid strand that displaces the entire second strand that was previously generated in step (b). The extension thereby provides a complete copy of the ds nucleic acid formed in step (b), as well as a single stranded nucleic acid that is complementary to the target nucleic acid (see, e.g., 12900 and 12930 in FIG. 12D). Optimal conditions for extension of the second primer using a SDP enzyme can be determined by one skilled in the art according to the size, composition and quantity of the target nucleic acid and the second primer. Typically, the extension is carried out under conditions that do not denature or otherwise impact the conformation of the target nucleic acid.

### (f) Repeating Each of Steps (b), (c), (d), and (e)

In some forms, following one or more steps to extend the hybridized second primer using a strand-displacing DNA polymerase enzyme, to provide an extended hybrid double-stranded (ds) DNA molecule and a displaced single-stranded (ss) DNA molecule that is complementary to the sequence of the target nucleic acid in step (e), the methods include repeating steps (c), (d) and (e) to provide a multiplicity of copies of a displaced single-stranded (ss) DNA molecule that is complementary to the target nucleic acid.

The steps of removing the hybrid domain from the ds nucleic acid by contacting the nucleic acid with a suitable removing reagent to create a single-stranded region, hybridizing a second oligonucleotide primer to the single-stranded region, and then extending the second primer using a suitable SDP enzyme can be repeated once or more, to provide a multiplicity of copies of a displaced single-stranded (ss) DNA molecule that is complementary to the sequence of the target nucleic acid.

In some forms, in the absence of an inhibitory factor, the sequence of steps (a), (d) and (c) are repeated until all available substrates are exhausted. Therefore, in some forms, the amount of a reagent, such as a first primer, a second primer, an endonuclease such as an RNase (e.g., an RNase H), and/or a SDP enzyme that is contacted with a target nucleic acid is configured to provide a desired amount of a displaced single-stranded (ss) DNA molecule that is complementary to the sequence of the target nucleic acid. In other forms, an inhibitor, such as an inhibitor of an RNase H enzyme, or an inhibitor of a SDP enzyme, are applied to stop the further repetition of steps (c), (d) and (c).

### B. Strand-Displacement Amplification of Double-Stranded Target DNA

Methods for strand-displacement amplification of a double-stranded target DNA molecule are provided. In some forms, when the methods include amplification based on a starting nucleic acid that is double-stranded, the methods employ one or more adapter handles that perform the function of providing a removable region on a single strand of the ds target. In this manner, an adapter performs an equivalent function to the “first primer” in the above methods. Accordingly, methods that are based on the same principle of creating a selectively removable sequence as a hybridization site for subsequent amplification steps based on a ds nucleic acid target are provided.

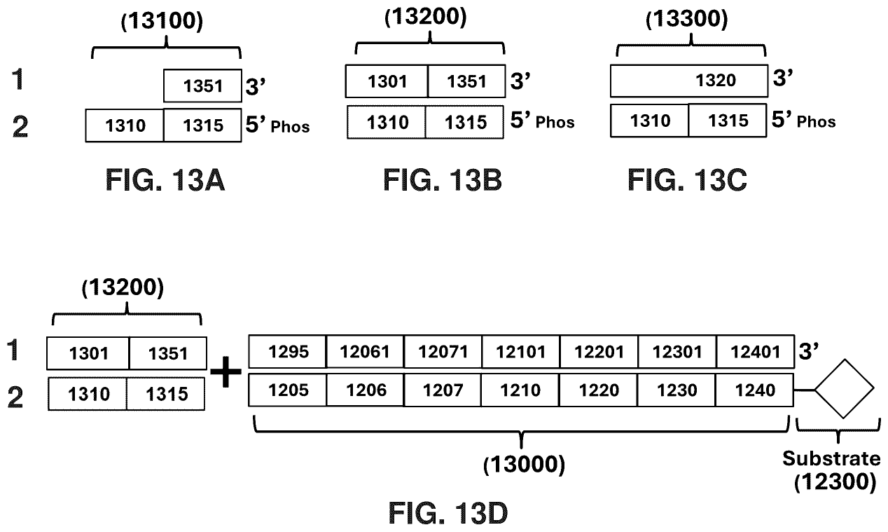

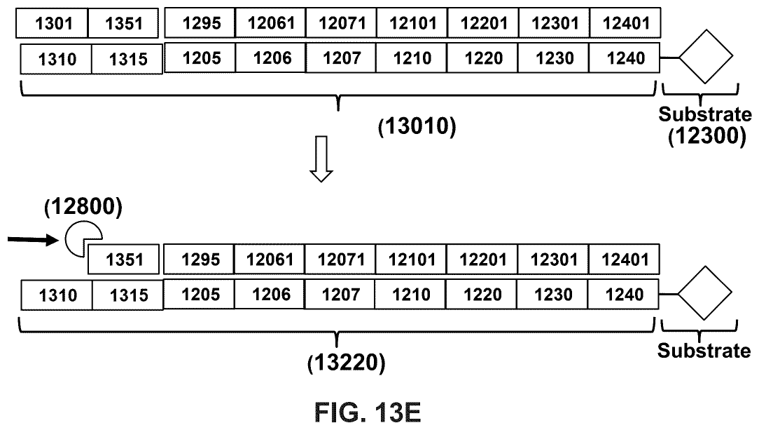

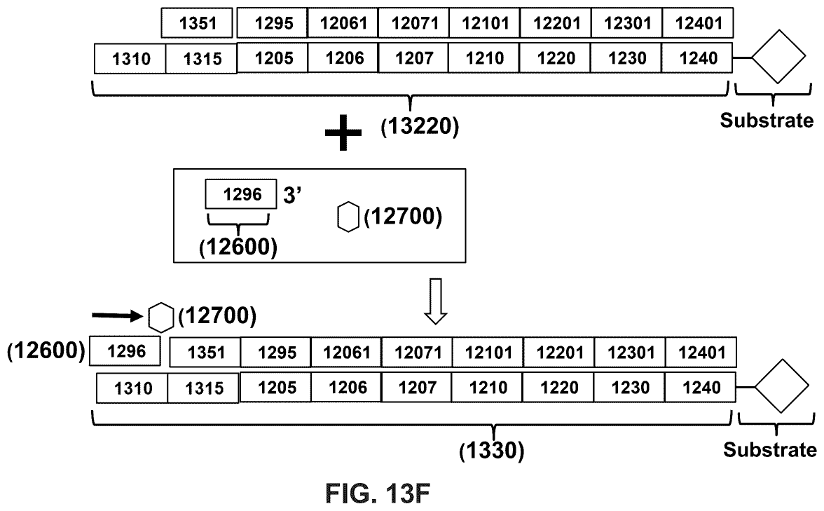

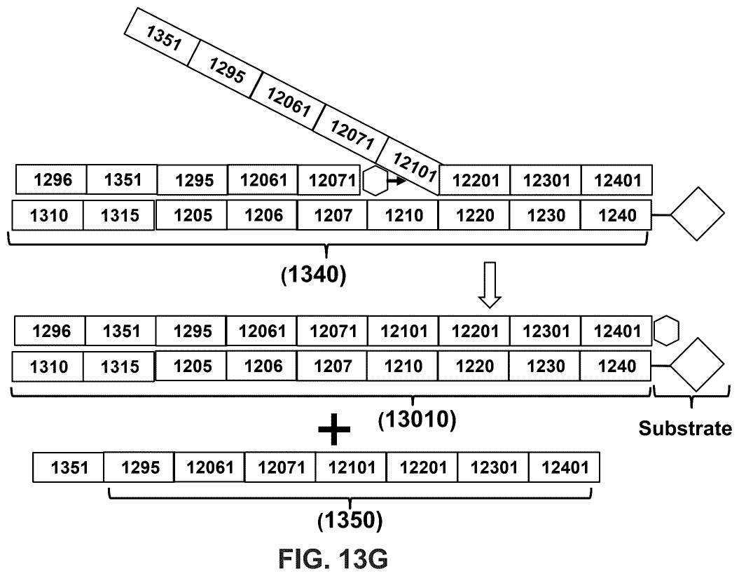

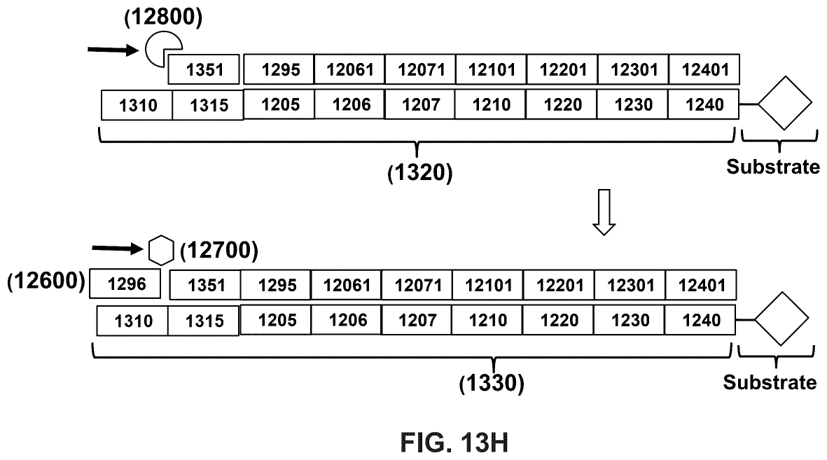

Different methodologies, based on the method steps (a)-(c), discussed above, implement different forms of adapter, according to the requirements of the user. Exemplary adapters include: (1) a pre-formed dsDNA adapter having a pre-formed removable region as a cleavable component; (2) a pre-formed, partly double-stranded adapter, having a 3′ overhang; and (3) an adapter formed in situ on the target ds nucleic acid, where a first adapter strand is linked to a single strand of the ds target, and then a second strand is hybridized to the first to form the ds adapter in situ. Different exemplary ds adapter structures are depicted in FIGS. 13A-13C.

Methods encompassing adapters according to each of these different design parameters are discussed in more detail, below. Typically, the methods include forming a combined first molecule from a target double stranded (ds) DNA molecule having a first DNA strand and a second DNA strand. In some forms, the methods include linking a first strand of an adapter to a free 3′ end of a target DNA strand that is conjugated to a substrate at the 5′ end. Typically, the linking involves forming a phosphor-diester bond with a 5′ phosphate moiety present on the first strand of the adapter.

### 1. Methods Employing a Pre-Formed Ds Nucleic Acid Adapter

In an exemplary form, a method for amplification of a double-stranded (ds) nucleic acid target molecule, such as a dsDNA molecule, includes a pre-formed ds nucleic acid adapter having a pre-formed removable region as a cleavable component.

In an exemplary form, the methods include one or more steps of:
(a) forming a combined first molecule from a target double stranded (ds) nucleic acid, such as a dsDNA molecule including a first DNA strand and a second DNA strand, the forming including:
   (i) linking to a 3′ end of the first DNA strand of the dsDNA molecule a nucleic acid adapter, the nucleic acid adapter including:
      (I) a first adapter strand, including:
         (i) a first region, including DNA nucleotides; and
         (ii) a second hybrid region, including RNA nucleotides, or DNA nucleotides including a unique target sequence, or inosine nucleotides;
   and
      (II) a second adapter strand including a DNA sequence complementary to all or part of the first strand;
      wherein the first adapter strand is hybridized to the second adapter strand;
   optionally wherein the second adapter strand is linked to the 3′ end of the second DNA strand via a 5′ phosphodiester linkage;
(b) removing the second hybrid region from the combined first molecule to form a truncated nucleic acid molecule including a region of ss nucleic acid, such as a truncated DNA molecule including a region of ssDNA;
(c) hybridizing a second primer to the region of ssDNA of the truncated DNA molecule to form a hybridized second molecule including a hybridized second primer,
wherein the second primer includes the second hybrid region including RNA nucleotides, DNA nucleotides including a unique target sequence, or inosine nucleotides;
(d) extending the hybridized second primer with a strand-displacing DNA polymerase to provide an extended hybrid dsDNA molecule and a displaced ssDNA molecule; and
(e) optionally repeating steps (b), (c) and (d) to provide a multiplicity of copies of a displaced single-stranded (ss) DNA molecule that includes the sequence of the second strand of the target ds nucleic acid.

In some forms, two or more of steps (a), (b), (c), and/or (d), are carried out following a single active step, for example, where a single reaction mixture including reagents necessary for two or more of steps (a), (b), (c), and/or (d), is used. In an exemplary form, a single reaction mixture including a ds adapter molecule, a second oligonucleotide primer, a strand displacing polymerase and an RNase enzyme is used, such that each of steps (a), (b), (c), and/or (d), are carried out following a single active step of contacting the target ds nucleic acid with the reaction mixture under suitable conditions for an isothermal strand displacement reaction to occur.

Each of these steps is described in greater detail, below.

(a) Forming a Combined First Molecule from Target Ds Nucleic Acid

Typically, the methods include one or more steps of forming a combined first molecule from a target double stranded (ds) nucleic acid, such as a dsDNA molecule, and a dsDNA adapter molecule.

Typically, a target ds nucleic acid molecule includes a first strand and a substantially complementary second strand, hybridized together to form a ds nucleic acid.

In some forms, a 5′ end of a second strand of a ds nucleic acid is conjugated to a substrate, such as an array, such that 3′ end of the second strand is free to link to a 5′ end of a second strand of an adapter. In some forms, a 5′ end of a first strand of a ds nucleic acid is free to link to a 3′ end of a first strand of an adapter.

For example, in some forms, 3′ end of a first strand of the adapter is linked to the 5′ end of a first strand of the dsDNA target, for example, where 5′ end of the second strand of the dsDNA target is conjugated to a substrate, such as an array.

An exemplary step including forming a combined first molecule from a target double stranded (ds) DNA molecule having a first DNA strand and a second DNA strand is depicted in FIG. 13D, where 5′ end of the second strand of the dsDNA (13000) is conjugated to a substrate (12300). In some forms, the combined first molecule is formed by linking to a 3′ end of the second DNA strand of the dsDNA molecule to a 5′ end of a second strand of the nucleic acid adapter, for example, by forming a phosphodiester linkage with 5′end of the second strand of the ds adapter bearing a phosphate molecule (see, e.g., 13200 in FIG. 13B).

### 1. Double-Stranded (Ds) Adapter Design

Adapter molecules, such as double-stranded (ds) adapter molecules for use in the described methods are provided. The ds adapter molecules typically include at least two at least partly complementary nucleic acid strands, a first strand and a second strand, hybridized together in the region of complementarity. Typically, each strand of the ds adapter includes at least one functional nucleic acid “domain”. Exemplary functional nucleic acid regions including a first DNA region and a second “hybrid” (removable) region.

Each strand of the ds adapter molecules typically includes from about 8 to about 200 nucleotides, inclusive, or any integer (or range of integers) of nucleotides in between the indicated values, for example, between about 16 to about 64 nucleotides, inclusive, or between about 12 to about 24 nucleotides, inclusive. In some forms, a first strand of an adapter includes about 16 nucleotides. In some embodiments, each of the functional nucleic acid “domains”, includes about 4, 5, 6, 7, 8, 9, 10, 11, 12, 13, 14, 15 or 16, or more nucleotides.

At least one strand of the ds adapter molecule typically includes more than a single type of nucleotide. For example, in some forms, the second strand of the ds adapter molecule includes only DNA, whereas the first strand of the ds adapter molecule includes both DNA and RNA, or DNA and inosine, or DNA and methylated DNA. In other forms, both the first and the second strand of the ds adapter molecule include only DNA nucleotides, for example, where a second strand includes a nucleotide sequence that functions to link to the target nucleic acid, and a first strand includes a hybrid region including a DNA nucleotide sequence that functions as an enzyme recognition sequence, such as a restriction endonuclease recognition sequence. Typically, the second strand of the ds adapter molecule primer includes a 5′ phosphate moiety that can form the link with 3′ end of the first strand of the target nucleic acid (see, e.g., 13200 in FIG. 13B).

a. First Hybrid (Removable) Strand of Ds Adapter

The first strand of the ds adapter molecule includes a “hybrid” domain including a removable nucleotide sequence that is or includes a substrate for an enzyme or reaction that digests and/or cleaves all or part of the domain from within a double-stranded nucleic acid. In some forms, the “hybrid” domain is the only domain within the first strand of the ds adapter molecule (see, e.g., (1320) in the first strand of the ds adapter depicted in 13300 of FIG. 13C). In other forms, the “hybrid” domain is one of two or more functional domains within the first strand of the ds adapter molecule (see, e.g., (1301) in the first strand of the ds adapter depicted in 13100 of FIG. 13B). Regardless of the number of functional domains present within a first strand of a ds adapter, the removable domain is typically orientated at the 5′ end of the first strand, such that removal of the domain will provide a truncated ds nucleic acid molecule, such as a truncated ds DNA molecule, with a 3′ single-stranded nucleic acid overhang, as depicted in 13220 of FIG. 13E)

In some forms, the hybrid domain of the first strand of the ds adapter molecule is formed entirely from RNA and includes a sufficient number of RNA nucleotides that, when removed, provide a region of single-stranded (“sticky-end”) 3′ nucleic acid overhang at the end of a truncated ds nucleic acid molecule, such as a truncated dsDNA molecule, that is sufficient for selective hybridization of a second oligonucleotide primer to the single-stranded overhang. In some forms, the hybrid domain includes about 4, 5, 6, 7, 8, 9, 10, 11, 12, 13, 14, 15 or 16, or more than 16 contiguous nucleotides. In an exemplary form, the hybrid domain includes 8 nucleotides. The design of the hybrid domain should include determination of a suitably large sequence to ensure effective removal of the domain by a corresponding enzyme or reagent.

b. Second Strand of Ds Adapter

Typically, the second strand of a ds adapter is the strand that, orientated 3′-5′, will bind to the free 3′ end of the second strand of a ds nucleic acid target molecule, such as a dsDNA target molecule (e.g., where 5′ end of the second strand of a dsDNA target molecule is conjugated to a substrate, such as (12300) in FIG. 13D).

Typically, the second strand of a ds adapter includes only DNA, having one or more functional domains. As depicted in FIGS. 13B and 13C, an exemplary second strand of a ds adapter includes two DNA “domains” (see, e.g., (1310) and (1315) in 13200 and 13300), including a 5′ phosphate on 5′ end of (1315). There is no need for the second strand of an adapter to include any nucleotide sequence that is complementary to any part of a target DNA molecule.

At least one functional domain of the second strand will provide a hybridization domain for a removable domain of the first strand. Therefore, a functional domain of the second strand of a ds adapter is typically formed entirely or partially from DNA and includes a sufficient number of nucleotides to impart specificity for hybridization to a removal domain of the first strand of the ds adapter.

In an exemplary form, as depicted in FIG. 13B, the second strand of a ds adapter includes two functional domains, including a first domain (1315) having a 5′ phosphate on the 5′ end, and a second functional domain (1310) at 3′ end of the second strand of the ds adapter that hybridizes with a removal domain of the first strand of the ds adapter. In an exemplary form, the second functional domain (1310) of the second strand of the ds adapter includes about 4, 5, 6, 7, 8, 9, 10, 11, 12, 13, 14, 15 or 16, or more than 16 contiguous nucleotides. In an exemplary form, the second domain of the second strand of the ds adapter includes 8 nucleotides. The design of the second domain of the second strand should include determination of a suitably unique sequence that is of sufficient length to ensure selective hybridization. Typically, the second domain of the second strand of the ds adapter is located at 3′ region of the second strand of the ds adapter.

### 2. Exemplary Double Stranded Adapters

Compositions of double stranded nucleic acid adapter molecules and use thereof according to the described methods are also described.

Exemplary double stranded nucleic acid adapter molecules include:
(a) a first adapter strand, including: (i) a first region, including DNA nucleotides; and (ii) a second hybrid region, including RNA nucleotides, or DNA nucleotides including a unique target sequence, or inosine nucleotides; and (b) a second adapter strand including a DNA sequence complementary to all or part of the first strand; wherein the first adapter strand is hybridized to the second adapter strand.

In some forms, the second adapter strand is linked to the 3′ end of the second DNA strand via a 5′ phosphodiester linkage. In some forms, the first adapter strand includes between about 8 and about 100 nucleotides, inclusive. For example in some forms, the first region of the first adapter strand includes between about four and about 30 nucleotides, inclusive, and/or the first region of the second adapter strand includes between about four and about 30 nucleotides, inclusive. In some forms, the second hybrid region includes between about four and about 30 nucleotides, inclusive. In some forms, the first region of the first adapter strand includes one or more locked nucleic acids (LNA) and/or the first region of the second adapter strand includes one or more locked nucleic acids (LNA).

In some forms, the second hybrid region includes RNA nucleotides. In other forms, the second hybrid region includes inosine nucleotides. In other forms, the second hybrid region includes a unique target sequence, and wherein the target sequence includes a nickase recognition sequence. In other forms, the second hybrid region includes a methylated GATC nucleotide sequence.

(b) Removing Second Hybrid Region from the Combined First Molecule

Following forming a combined first molecule from a target double stranded (ds) DNA molecule and a dsDNA adapter molecule in step (a), the methods include one or more steps of removing the hybrid region from the combined first molecule to form a truncated combined molecule including a region of ssDNA.

Typically, the methods remove the hybrid region from the 5′ end of the first strand of the combined molecule by contacting the combined molecule with a suitable reagent to selectively remove the hybrid region. For example, if the hybrid region of the adapter molecule includes RNA, the step of removing the hybrid region includes contacting the combined first molecule with an RNase enzyme, such as RNase H. FIG. 13E depicts an exemplary RNase H (12800) mediated removal of the hybrid region (1301) of an extended hybrid double-stranded (ds) DNA molecule (13010) to yield a truncated combined molecule (13220) including a “sticky end” region of ssDNA (1310) at 3′ end of the second strand of the combined first molecule.

Optimal conditions for removal of a removable hybrid region of a combined first molecule, such as RNA, using a suitable reagent, such as RNase H, can be determined by one skilled in the art according to the size, composition and quantity of the hybrid region. Typically, the removal is carried out under conditions that do not denature or otherwise impact the conformation of the target nucleic acid.

### (c) Hybridizing a Second Primer to the Target DNA

Following removal of the hybrid region of the combined first molecule in step (b), the methods include one or more steps of hybridizing a second primer to the region of ssDNA of the truncated DNA molecule to form a hybridized second molecule including a hybridized second primer. Typically, the second primer includes the second hybrid region including RNA nucleotides, DNA nucleotides including a unique target sequence, or inosine nucleotides.

The second oligonucleotide primer typically is or includes all or part of the removable hybrid region of the adapter molecule, including removable components. In an exemplary form, the second primer includes only RNA nucleotides.

Typically, the methods hybridize the second primer formed entirely of hybrid, selectively removable nucleotides to the truncated DNA molecule to form a ds nucleic acid that is amenable to amplification by a strand-displacing polymerase.

The second primer typically hybridizes to the partially single stranded, truncated dsDNA molecule in a manner that leaves a nick (i.e., a single-stranded break) at 3′ end of the primer (see, e.g., 1296 of 1330 in FIG. 13F).

Optimal conditions for hybridizing a second primer to a truncated dsDNA molecule can be determined by one skilled in the art according to the size, composition and quantity of the target nucleic acid and the second primer. Typically, hybridization is carried out under conditions that do not denature or otherwise impact the conformation of the target nucleic acid.

### (d) Extending the Hybridized Second Primer

Following hybridizing a second primer to the region of ssDNA of the truncated DNA molecule to form a hybridized second molecule including a hybridized second primer in step (c), the methods include one or more steps of extending the hybridized second primer with a strand-displacing DNA polymerase to provide an extended hybrid dsDNA molecule and a displaced ssDNA molecule.

The extension reaction in step (d) requires a strand displacing polymerase; SDP enzyme). In some forms, extension is initiated by 5′ nick at 3′ region of the second primer to yield a new (i.e., “third”) nucleic acid strand that displaces the entire first strand of the hybridized second molecule. The extension thereby provides a complete copy of the first strand of the ds target nucleic acid and also yields a target ds nucleic that can be re-primed for subsequent amplification step(s) (see, e.g., 1340, which provides 13010 and 1350 in FIG. 13G).

Optimal conditions for extension of the second primer using a SDP enzyme can be determined by one skilled in the art according to the size, composition and quantity of the target nucleic acid and the second primer. Typically, the extension is carried out under conditions that do not denature or otherwise impact the conformation of the target nucleic acid.

### (e) Repeating Each of Steps (b), (c), and (d)

In some forms, following one or more steps to extend the hybridized second primer using a strand-displacing DNA polymerase enzyme, to provide an extended hybrid double-stranded (ds) DNA molecule and a displaced single-stranded (ss) DNA molecule that includes the sequence of the first strand of the ds target nucleic acid in step (d), the methods include repeating steps (b), (c) and (d) to provide a multiplicity of copies of a displaced single-stranded (ss) DNA molecule that includes the sequence of the first strand of the ds target nucleic acid.

The steps of removing the hybrid region from the first extended molecule by contacting the removable region with a suitable removing reagent to create a single-stranded 3′ “sticky end” region, hybridizing a second oligonucleotide primer to the single-stranded 3′ “sticky end” region, and then extending the second primer using a suitable SDP enzyme can be reproduced once or more, to provide a multiplicity of copies of a displaced single-stranded (ss) DNA molecule that includes the sequence of the first strand of the ds target nucleic acid.

In some forms, in the absence of an inhibitory factor, the sequence of steps (b), (c) and (d) are repeated, e.g., until all available substrates are exhausted. Therefore, in some forms, the amount of a reagent, such as a second primer, an RNaseH, and/or a SDP enzyme that is contacted with a first extended molecule is configured to provide a desired amount of a displaced single-stranded DNA that includes the sequence of the second strand of the ds target nucleic acid. In other forms, an inhibitor, such as an inhibitor of an RNaseH enzyme, or an inhibitor of a SDP enzyme, are applied to stop the further repetition of steps (b), (c) and (d).

### 3. Methods Employing Pre-Formed, Partly Double-Stranded Adapter

In some forms, a method for amplification of a double-stranded (ds) target DNA molecule includes a pre-formed, partially double-stranded adapter that does not include a removable region. When the methods include a partially double-stranded adapter molecule, the methods can effectively skip one or more steps of removing a removable region from a fully-double-stranded adapter, as described above.

Therefore, in some forms, the methods include forming a combined molecule from a target ds nucleic acid (such as dsDNA) linked with a pre-formed, partially double-stranded adapter that includes a region of complementary, double-stranded nucleic acids, and a region of single-stranded nucleic acids. The region of single-stranded nucleotides on the adapter introduce a single stranded “overhang” region that provides a hybridization site for a second primer that is formed entirely from, or includes a removable component.

In an exemplary form, methods for amplification of a single strand of a target double stranded DNA molecule using a pre-formed, partially double-stranded adapter include one or more steps of:
(a) forming a combined first molecule having a 3′overhang from a target double stranded (ds) DNA molecule having a first DNA strand and a second DNA strand, the forming including: linking to a 3′ end of the second DNA strand of the dsDNA molecule a 5′ end of a second strand of a partially ds nucleic acid adapter, the partially ds nucleic acid adapter including:
   (i) a first adapter strand, consisting of a first region of DNA nucleotides; and
   (ii) a second adapter strand, including a DNA sequence complementary to the first region of DNA nucleotides of the first strand and a second region of DNA nucleotides, wherein the second region includes at least 5 nucleotides;
wherein the second adapter strand is hybridized to the first region of DNA nucleotides of the first adapter strand to form the partially ds nucleic acid adapter including a 3′ overhang, and
wherein 3′ overhang includes the second region of DNA nucleotides of the first adapter strand;
optionally wherein the second adapter strand is linked to the 3′ end of the second DNA strand via a 5′ phosphodiester linkage;
(b) hybridizing a second primer to 3′ overhang of the combined first molecule to form a hybridized second molecule including a hybridized second primer,
wherein the second primer includes a sequence complementary to the second region of DNA nucleotides of the first adapter strand, and
wherein the second primer includes RNA nucleotides, DNA nucleotides including a unique target sequence, or inosine nucleotides;
(c) extending the hybridized second primer with a strand-displacing DNA polymerase to provide an extended hybrid dsDNA molecule and a displaced DNA molecule; and
(d) optionally removing the second primer from the extended hybrid dsDNA molecule to form a truncated dsDNA molecule including a region of ssDNA,
wherein the region of ssDNA includes the second region of DNA nucleotides of the first adapter strand; and
(c) further optionally repeating step (c).

Each of these steps is described in greater detail, below.

(a) Forming a Combined First Molecule from Target Ds DNA

The methods include one or more steps of forming a combined first molecule from a target double stranded (ds) DNA molecule and a partially double stranded adapter molecule.

Typically, a target ds nucleic acid molecule includes a first strand and a substantially complementary second strand, hybridized together to form a ds nucleic acid. In some forms, a target ds DNA molecule includes a first strand and a second strand, where 5′ end of the second strand of the dsDNA target is conjugated to a substrate, such as an array, with 3′ end of the second strand of the target ds DNA molecule free to link to 5′ end of a second strand of a partially ds DNA adapter molecule.

When the methods employ a partially double stranded adapter that includes a single-stranded “sticky end” overhang, the methods include one or more steps of forming a combined first molecule having a 3′overhang from a target double stranded (ds) DNA molecule having a first DNA strand and a second DNA strand, by linking to a partially dsDNA adapter molecule.

Typically, the step of forming a combined first molecule includes linking to a 3′ end of the second DNA strand of the dsDNA molecule a 5′ end of a second adapter strand of a partially ds nucleic acid adapter. In some forms, a second strand of the partially ds nucleic acid adapter includes a 5′ phosphate moiety. Therefore, in some forms, forming a combined first molecule includes linking to a 3′ end of the second DNA strand of the dsDNA molecule a 5′ end of a second adapter strand of a partially ds nucleic acid adapter via a 5′ phosphodiester linkage.

An exemplary combined first molecule having a 3′overhang formed from a target double stranded (ds) DNA molecule having a first DNA strand and a second DNA strand with a partially double stranded adapter molecule having a 3′ overhang is depicted as 13220 in FIG. 13E.

### 1. Partially Double-Stranded Adapter Design

Design parameters for partially double-stranded adapters that include a single-stranded “sticky end” overhang for use in the described methods are provided.

Partially ds adapter molecules, such as adapter molecules having a 3′ overhang for use in the described methods are provided. The partially ds adapter molecules typically include at two at least partly complementary nucleic acid strands, a first strand and a second strand, hybridized together in the region of complementarity. Typically, each strand of the partially ds adapter includes at least one functional nucleic acid “domain”. Typically, the second adapter strand includes one more domain that the first adapter strand, such that the combined strands form a 3′ overhang region (see, for example, 13100 depicted in FIG. 13A).

Each strand of a partially ds adapter molecule typically includes from about 8 to about 200 nucleotides, inclusive, or any integer (or range of integers) of nucleotides in between the indicated values, for example, between about 16 to about 64 nucleotides, inclusive, or between about 12 to about 24 nucleotides, inclusive.

a. First (Short) Strand of Partially Ds Adapter

The first strand of the partially ds adapter molecule is shorter than the second strand of the partially ds adapter. Therefore, the first strand of the partially ds adapter is typically completely or substantially hybridized to a complementarity nucleotide sequence in the second strand (see, e.g., (1351) in the first strand of the partially ds adapter depicted in 13100 of FIG. 13A). In other forms, the “hybrid” domain is one of two or more functional domains within the first strand of the ds adapter molecule (see, e.g., (1301) in the first strand of the ds adapter depicted in 13100 of FIG. 13B).

In some forms, the first strand of the partially ds adapter includes one or more functional domains. In some forms, the first strand of the partially ds adapter is configured to bind to the free 5′ end of the first strand of the ds target nucleic acid such that, when combined with a target ds DNA molecule, the shorter size of the first strand of the adapter relative to the second strand of the adapter will provide a truncated ds DNA molecule with a 3′ sticky end, as depicted in 13220 of FIG. 13E or FIG. 13F).

In some forms, the first strand of a partially ds adapter molecule includes from about 8 to about 100 nucleotides, inclusive, or any integer (or range of integers) of nucleotides in between the indicated values, for example, between about 16 to about 64 nucleotides, inclusive, or between about 12 to about 24 nucleotides, inclusive. In an exemplary form, the first strand of a partially ds adapter includes about 4, 5, 6, 7, 8, 9, 10, 11, 12, 13, 14, 15 or 16, or more than 16 contiguous nucleotides. In an exemplary form, the first strand of a partially ds adapter includes 8 nucleotides. The design of the first and second strands of the partially ds adapter typically includes a suitably large difference between the number of nucleotides in the first and second strands to ensure 3′ sticky end includes a sufficient number and sequence of nucleotides to facilitate specific and effective hybridization of a second primer to the 3′ sticky end. For example, in some forms, the first strand of a partially ds adapter molecule is smaller than the second strand of a partially ds adapter by at least about 8 to about 100 nucleotides, inclusive, or any integer (or range of integers) of nucleotides in between the indicated values, for example, between about 16 to about 64 nucleotides, inclusive, or between about 12 to about 24 nucleotides, inclusive. In an exemplary form, the first strand of a partially ds adapter is smaller than the second strand of a partially ds adapter by 4, 5, 6, 7, 8, 9, 10, 11, 12, 13, 14, 15 or 16, or more than 16 contiguous nucleotides.

b. Second (Overhang) Strand of Partially Ds Adapter

Typically, the second strand of a partially ds adapter is the strand that, orientated 3′-5′, will bind to the free 3′ end of the second strand of a dsDNA target molecule (e.g., where the 5′ end of the second strand of a dsDNA target molecule is conjugated to a substrate, such as (12300) in FIG. 13D).

Typically, the second strand of a partially ds adapter includes only DNA, having one or more functional domains. As depicted in FIG. 13A, an exemplary second strand of a partially ds adapter includes two DNA “domains” (see, e.g., (1310) and (1315) in 13100 in FIG. 13A), including a 5′ phosphate on 5′ end of (1315). There is no need for the second strand of an adapter to include any nucleotide sequence that is complementary to any part of a target DNA molecule.

At least one functional domain of the second strand will form part of, or will provide a 3′ sticky end that facilitates hybridization to a second oligonucleotide primer. Therefore, a second strand of a partially ds adapter is typically formed entirely or partially from DNA and includes a sufficient number of nucleotides to impart specificity for hybridization to a a second oligonucleotide primer.

In some forms, the second strand of a partially ds adapter molecule includes from about 8 to about 100 nucleotides, inclusive, or any integer (or range of integers) of nucleotides in between the indicated values, for example, between about 16 to about 64 nucleotides, inclusive, or between about 12 to about 24 nucleotides, inclusive. In an exemplary form, the second strand of a partially ds adapter includes about 4, 5, 6, 7, 8, 9, 10, 11, 12, 13, 14, 15 or 16, 20, 30 or more than 30 contiguous nucleotides. In an exemplary form, the second strand of a partially ds adapter includes 16 nucleotides. The design of the first and second strands of the partially ds adapter typically includes a suitably large difference between the number of nucleotides in the first and second strands to ensure 3′ sticky end includes a sufficient number and sequence of nucleotides to facilitate specific and effective hybridization of a second primer to 3′ sticky end. For example, in some forms, the second strand of a partially ds adapter molecule is larger than the first strand of a partially ds adapter by at least about 8 to about 100 nucleotides, inclusive, or any integer (or range of integers) of nucleotides in between the indicated values, for example, between about 16 to about 64 nucleotides, inclusive, or between about 12 to about 24 nucleotides, inclusive. In an exemplary form, the second strand of a partially ds adapter is larger than the second strand of a partially ds adapter by 4, 5, 6, 7, 8, 9, 10, 11, 12, 13, 14, 15 or 16, or more than 16 contiguous nucleotides.

### 2. Exemplary Partially Double Stranded Adapters

Compositions of partially double stranded nucleic acid adapter molecules and use thereof according to the described methods are also described. Exemplary partially double stranded nucleic acid adapter molecules include:
(i) a first adapter strand, consisting of a first region of DNA nucleotides; and
(ii) a second adapter strand, including a DNA sequence complementary to the first region of DNA nucleotides of the first strand and a second region of DNA nucleotides,
wherein the second region includes at least 5 nucleotides;
wherein the second adapter strand is hybridized to the first region of DNA nucleotides of the first adapter strand to form the partially ds nucleic acid adapter including a 3′ overhang, and wherein 3′ overhang includes the second region of DNA nucleotides of the first adapter strand.

In some forms, the second adapter strand is linked to the 3′ end of the second DNA strand via a 5′ phosphodiester linkage. In some forms, the first adapter strand includes between about 8 and about 100 nucleotides, inclusive. For example in some forms, the first region of the first adapter strand includes between about four and about 30 nucleotides, inclusive, and/or the first region of the second adapter strand includes between about four and about 30 nucleotides, inclusive. In some forms, the second hybrid region includes between about four and about 30 nucleotides, inclusive. In some forms, the first region of the first adapter strand includes one or more locked nucleic acids (LNA) and/or the first region of the second adapter strand includes one or more locked nucleic acids (LNA).

In some forms, the second hybrid region includes RNA nucleotides. In other forms, the second hybrid region includes inosine nucleotides. In other forms, the second hybrid region includes a unique target sequence, and wherein the target sequence includes a nickase recognition sequence. In other forms, the second hybrid region includes a methylated GATC nucleotide sequence.

### (b) Hybridizing a Second Primer to the Combined First Molecule

Following forming a combined first molecule having a 3′overhang from a target double stranded (ds) DNA molecule having a first DNA strand and a second DNA strand in step (a), the methods include one or more steps for hybridizing a second oligonucleotide primer to the region of ssDNA of the combined first molecule having a 3′overhang, to form a hybridized second molecule including a hybridized second primer.

The second primer includes a nucleotide sequence that selectively and specifically hybridizes to the ss region of the combined first molecule formed by 3′overhang. Typically, the second primer is or includes a removable hybrid region, including removable components such as RNA nucleotides, DNA nucleotides including a unique target sequence, or inosine nucleotides. In an exemplary form, the second primer includes only RNA nucleotides.

The second primer typically hybridizes to 3′overhang of the combined first molecule in a manner that leaves a nick (i.e., a single-stranded break) at 3′ end of the primer. Therefore, the methods hybridize the second primer to 3′overhang of the combined first molecule to form a hybridized second molecule including a hybridized second primer that is amenable to amplification by a strand-displacing polymerase (see, e.g., 1296 of 1330 in FIG. 13F).

Optimal conditions for hybridizing a second primer to 3′overhang of the combined first molecule can be determined by one skilled in the art according to the size, composition and quantity of 3′overhang of the combined first molecule and the second primer. Typically, hybridization is carried out under conditions that do not denature or otherwise impact the conformation of the target nucleic acid.

### (c) Extending the Hybridized Second Primer

Following hybridizing a second oligonucleotide primer to the region of ssDNA of the combined first molecule having a 3′overhang, to form a hybridized second molecule including a hybridized second primer in step (b), the methods include one or more steps of extending the hybridized second primer with a strand-displacing DNA polymerase to provide an extended hybrid dsDNA molecule and a displaced ssDNA molecule.

The extension reaction in step (c) requires a strand displacing polymerase; SDP enzyme). The extension initiated by 5′ nick at 3′ region of the second primer yields a new (i.e., “third”) nucleic acid strand that displaces the entire first strand of the hybridized second molecule. The extension thereby provides a complete copy of the first strand of the ds target nucleic acid, as well as a single stranded nucleic acid that is or includes the entire second strand of the target ds nucleic acid (see, e.g., 1340, which provides 13010 and 1350 in FIG. 13G).

Optimal conditions for extension of the second primer using a SDP enzyme can be determined by one skilled in the art according to the size, composition and quantity of the target nucleic acid and the second primer. Typically, the extension is carried out under conditions that do not denature or otherwise impact the conformation of the target nucleic acid.

### (d) Repeating Each of Steps (b) and (c)

In some forms, following one or more steps to extend the hybridized second primer using a strand-displacing DNA polymerase enzyme, to provide an extended hybrid double-stranded (ds) DNA molecule and a displaced single-stranded (ss) DNA molecule that includes the sequence of the first strand of the ds target nucleic acid in step (c), the methods include repeating steps (b), and (c) to provide a multiplicity of copies of a displaced single-stranded (ss) DNA molecule that includes the sequence of the first strand of the ds target nucleic acid.

The steps of hybridizing a second oligonucleotide primer to the single-stranded 3′ “sticky end” overhang, and then extending the second primer using a suitable SDP enzyme can be reproduced once or more, to provide a multiplicity of copies of a displaced single-stranded (ss) DNA molecule that includes the sequence of the first strand of the ds target nucleic acid.

In some forms, in the absence of an inhibitory factor, the sequence of steps (b), and (c) are repeated, e.g., until all available substrates are exhausted. Therefore, in some forms, the amount of a reagent, such as a second primer, an RNaseH, and/or a SDP enzyme that is contacted with a first extended molecule is configured to provide a desired amount of a displaced single-stranded DNA that includes the sequence of the second strand of the ds target nucleic acid. In other forms, an inhibitor, such as an inhibitor of an RNaseH enzyme, or an inhibitor of a SDP enzyme, are applied to stop the further repetition of steps (b), (c) and (d).

4. Methods of Forming an Adapter In Situ on the Target dsDNA

In some forms, a method for amplification of a double-stranded (ds) target DNA molecule using an adapter molecule to initiate the step-wise removal and hybridization of a hybrid primer includes one or more preliminary steps of forming the adapter from two single stranded oligonucleotides that are hybridized together to form a ds or partially ds adapter, as described for use in the methods above.

Therefore, any of the described methods that employ a fully or partially double-stranded adapter can also include one or more steps, prior to step (a), for forming an adapter. Steps of forming an adapter can include: (1) binding to a free 3′ end of a second strand of a ds nucleic acid, a 5′ end of a second oligonucleotide strand, for example, where the second oligonucleotide strand includes a 5′ phosphate; and (2) hybridizing to the second oligonucleotide strand a first oligonucleotide strand, such that the hybridizing forms a partly or fully ds nucleic acid adapter, as described in the methods above. Therefore, in some forms, the second oligonucleotide strand becomes the second adapter strand and the first oligonucleotide strand becomes the first adapter strand.

Therefore, in some forms, the methods include forming combined molecule from a ds target nucleic acid (such as dsDNA) by step-wise addition of a 5′ end of a second adapter strand to a free 3′ end of a second strand of a target nucleic acid, and hybridizing a first adapter strand to second adapter strand. The first and second oligonucleotides include a region of complementary. In some forms, the first oligonucleotide is formed entirely from, or includes a removable component. In other forms, the first oligonucleotide is smaller than the second oligonucleotide, such that hybridization introduce a single stranded “overhang” region that provides a hybridization site for a second primer.

In an exemplary form, methods for amplification of a single strand of a target double stranded DNA molecule include one or more steps of:
(a) forming a combined first molecule from a target double-stranded (ds) DNA molecule and a nucleic acid adapter,
wherein the target dsDNA molecule includes a first DNA strand and a second DNA strand, the forming including:
   (i) linking to a 3′ end of the second DNA strand of the dsDNA molecule a 5′ end of a second strand of the nucleic acid adapter, to form a linked second adapter strand, and
   (ii) hybridizing a first strand of the nucleic acid adapter to the linked second adapter strand to form the combined first molecule,
   wherein the first strand of the nucleic acid adapter includes
      (I) a first region including DNA nucleotides; and
      (II) a second hybrid region, including RNA nucleotides, DNA nucleotides including a unique target nucleotide sequence, or inosine nucleotides, or a methylated GATC nucleotide sequence,
wherein the first strand of the nucleic acid adapter includes a nucleotide sequence complementary to all or part of the second strand of the nucleic acid adapter;
optionally wherein a 5′ end of the second strand of the nucleic acid adapter is linked to a 3′ end of the second DNA strand of the dsDNA molecule via a phosphodiester linkage;
(b) removing the second hybrid region from the combined first molecule to form a truncated DNA molecule including a region of ssDNA;
(c) hybridizing a second primer to the region of ssDNA of the truncated DNA molecule to form a hybridized second molecule including a hybridized second primer,
wherein the second primer includes the second hybrid region including RNA nucleotides, DNA nucleotides including a unique target sequence, or inosine nucleotides;
(d) extending the hybridized second primer with a strand-displacing DNA polymerase to provide an extended hybrid dsDNA molecule and a displaced DNA molecule.

### C. Target Nucleic Acids

The methods provide one or more nucleic acids that include the nucleotide sequence of a target nucleic acid, or a sequence complementary to a target nucleic acid.

A target nucleic acid can be a double stranded or single stranded nucleic acid. In some forms, a target nucleic acid includes a spatial barcode, a capture domain, and one or more one or more functional domains (e.g., a primer binding site (e.g., a sequencing primer binding site), an amplification domain (e.g., a domain suitable for amplifying the nucleic acid using one or more primers)). In some forms, a target nucleic acid includes a spatial barcode, a unique molecular identifier, a capture domain, and one or more one or more functional domains. In some forms, a target nucleic acid includes a spatial barcode, a unique molecular identifier, a capture domain, a sequence corresponding to a target analyte (e.g., cDNA, ligated RTL probes, an intermediate agent), and one or more functional domains (e.g., a sequencing primer binding site, an amplification domain). In some forms, a target nucleic acid is a single-stranded (ss) nucleic acid including one or more functional domains.

Exemplary functional domains include one or more priming sites for a sequencing primer. As used herein, the terms “sequencing primer binding site” and “priming site for a sequencing primer” are used interchangeably. Exemplary sequencing primer binding sites include a nucleic acid sequence that selectively hybridizes to a first, second or further sequencing primer, such as a first, second or further commercially-available sequencing primer. An exemplary first sequencing primer binding site is a Read 2S domain. An exemplary second sequencing primer binding site is a Read 1T domain. Therefore, in some forms, functional domains include a sequence corresponding to a Read 2S domain and/or a sequence corresponding to a Read 1T domain.

In some forms, functional domains within a target nucleic acid include a Left-hand side RNA templated ligation domain, a Right-hand side RNA templated ligation domain, a capture domain, a unique molecular identifier (UMI), a spatial barcode and/or a Read 1T domain. In some forms, a target nucleic acid is conjugated to a substrate. In some forms, the substrate is part of, or includes an array. An exemplary substrate is glass slide. Typically, when the target nucleic acid is conjugated or otherwise attached to a substrate, the target nucleic acid is conjugated via its 5′ end to the substrate. For example, when the target nucleic acid is double stranded, a first strand of the ds target nucleic acid is conjugated via its 5′ end to a substrate.

An exemplary target DNA molecule is one or more of a plurality of arrayed capture probes. In some forms, the target DNA molecule includes one or more priming sites for a sequencing primer. Typically, the target DNA molecule includes a sequence corresponding to an analyte (e.g., RNA, DNA) derived from a tissue sample, such as a tissue sample derived from a mammalian subject. An exemplary subject is a human or mouse.

In some forms, the target DNA molecule includes a spatial barcode and/or a DNA sequence of at least a portion of a target nucleic acid analyte, or a complement thereof. Typically, the target nucleic acid analyte includes a nucleic acid, optionally selected from genomic DNA, RNA, synthetic DNA, or synthetic RNA. In some forms, the target nucleic acid analyte is derived from a biological sample. Exemplary target nucleic acid analytes include an RNA selected from the group consisting of small interfering RNA (siRNA), microRNA (miRNA), P-element-induced wimpy testis (PIWI)-interacting RNA (piRNA), small nucleolar RNA (snoRNA), small nuclear RNA (snRNA), messenger RNA (mRNA), ribosomal RNA (rRNA), long non-coding RNAs (lncRNA), and transfer RNA (tRNA). In certain forms, the RNA includes mRNA.

In some forms, the target DNA molecule is directly or indirectly conjugated to a substrate or matrix, such as a solid support or a gel. In some forms, an array includes a multiplicity of capture probes directly or indirectly attached to a substrate. In some forms, the target DNA molecule is conjugated to the substrate or matrix via a first cleavable linker. Exemplary first cleavable linkers are selected from a photocleavable linker, UV-cleavable linker, or an enzyme-cleavable linker. In some forms, the first cleavable linker is an enzyme-cleavable linker.

In some forms, the target DNA molecule includes a spatial barcode and/or at least a portion of a target nucleic acid analyte or a complement thereof. In non-limiting forms, a substrate is conjugated to 5′end of the target DNA molecule; and a first primer binding site is located at the 3′ end of the target DNA molecule.

In some forms, a target dsDNA molecule includes a spatial barcode and/or at least a portion of a target nucleic acid analyte or a complement thereof, and a substrate is conjugated to 5′end of a first strand of the target DNA molecule; and/or the second strand of the double stranded nucleic acid adapter is linked to the 3′ end of the first strand of the target dsDNA molecule. In certain forms, a target DNA molecule includes one or more of a unique molecular identifier (UMI); a spatial barcode; and a capture domain. In certain forms, a target DNA molecule includes one or more of a unique molecular identifier (UMI); a sequence of at least a portion of a target analyte, or a complement thereof; and a spatial barcode.

Typically, the target DNA molecule includes a nucleotide sequence of a coding region of an mRNA, or a complement thereof. In some forms, the target DNA molecule includes a region complementary to a poly(A) sequence, or a complement thereof. In some forms, the target DNA molecule includes one or more functional domains. An exemplary functional domain includes an amplification domain or a primer-binding site.

### D. Removable “Hybrid” Domains

The described methods for strand-displacement amplification of a single or double-stranded target DNA molecule require the selective removal of at least one component of the first primer or adapter molecule, such that a corresponding removable primer can be hybridized to the single stranded DNA that results from the removal, and the amplification reaction can proceed indefinitely with subsequent rounds of hybridization, extension and removal. Therefore, the methods include selection of a removable component for including within the removable domain.

Typically the removable domain of includes at least about 4, 5, 6, 7, 8, 9, 10, 11, 12, 13, 14, 15 or 16, or more than 16 contiguous nucleotides. In an exemplary form, the removable domain of includes 8 nucleotides. The design of the removable domain typically includes determination of a suitably unique sequence that is of sufficient length to ensure selective hybridization. Typically, the second domain of the removable domain is located at the 3′ region of a molecule.

### 1. Selection of Hybrid Components

Removable nucleotides for use within removable “hybrid” domains and corresponding reagents for use with the methods are described. A hybrid domain typically includes specific nucleotides that are selectively and specifically removed upon contact with a suitable enzyme or reagent. For example, a hybrid domain within a first oligonucleotide primer molecule, an adapter molecule, or a second oligonucleotide primer molecule includes a sequence of nucleotides that are selectively and specifically removed upon contacting the first oligonucleotide primer molecule or the adapter molecule or the second oligonucleotide primer molecule with a suitable enzyme or reagent. Typically, the selection of removable nucleotides and removing agent is carried out to ensure selective removal of the removable domain occurs at a controlled time and location. In some forms, selective nuclease enzymes that catalyze the hydrolysis of specific types and/or sequences nucleotide linkages are suitable removing agents.

### (i) RNA/RNase Enzymes

In some forms, the removable nucleotides within the hybrid domain includes RNA nucleotides that are selectively and specifically digested upon exposure to Ribonucleases (RNase) enzymes. Ribonucleases (RNases) include a superfamily of nucleases that catalyze the hydrolysis of 3′,5′-phosphodiester linkages in RNA. Typically, RNase catalysis involves formation of nucleoside 2′,3′-cyclic phosphate intermediates, and generation of a 3′-phosphate group. An exemplary RNase enzyme suitable for use in the described methods includes RNase. RNase H will only digest RNA when it is annealed to DNA, thereby preventing undesired digestion and removal of RNA. Therefore, in some forms, the removable domain includes RNA nucleotides that are selectively removed, cleaved or digested upon exposure to an RNase H enzyme.

### (ii) Inosine/Endonuclease Enzymes

In some forms, removable nucleotides within the hybrid removable domain includes inosine nucleotides that are selectively and specifically digested upon exposure to endonucleases enzymes. Inosines are normal residues in certain RNAs introduced by specific deaminases. An inosine base can be used as a hybridization probe and form base-pairs with dA, dC, dG or dT residues. Endonuclease V enzymes, such as human endonuclease V (ENDOV), as well as Escherichia coli endonuclease V are highly active ribonucleases specific for inosine in RNA. ENDOV will selectively and specifically digest inosine bases within DNA when in duplex, and only digest the Inosine-containing strand. Therefore, in some forms, the removable domain includes inosine bases that are selectively removable upon exposure to an endonuclease V enzyme.

(iii) DNA/Restriction Endonuclease

In some forms, removable nucleotides within the hybrid removable domain includes a DNA nucleotide sequence that is selectively and specifically digested upon exposure to one or more restriction endonucleases (RE) enzymes. Restriction enzymes, also called restriction endonucleases, recognize a specific sequence of nucleotides in double stranded DNA and cut the DNA at a specific location. They are highly selective to the sequence and conformation of DNA molecules. An exemplary RE enzyme suitable for use in the described methods includes an RE having a unique cut site that can be designed into the 3′ end of the target nucleic acid. RE recognition sequences are well known in the art. Therefore, in some forms, the removable domain includes a DNA nucleotide sequence that is selectively cleaved upon exposure to an RE enzyme. In some forms, the RE enzymes are DNA/sequence specific nickase enzymes. Nicking endonucleases are a type of RE that recognizes short specific DNA sequence and cleave DNA at a fixed position relatively to the recognition sequence. However, unlike restriction endonucleases, nicking endonucleases cleave only one predetermined DNA strand.

### (iv) DpnI Enzyme/GmATC Sequences

In some forms, removable nucleotides within the hybrid removable domain include methylated nucleotide sequences that are selectively and specifically digested upon exposure to DpnI enzymes. Therefore, in some forms, the removable domain includes a methylated guanine, for example, a G^{methyl} ATC nucleotide sequence, that is selectively removed/digested upon exposure to a DpnI enzyme.

### 2. Exemplary Hybrid Domain/Removal Agents

In exemplary forms, removing the hybrid region includes contacting the second hybrid region with an enzyme that selectively degrades the second hybrid region. For example, in some forms, the hybrid region includes RNA nucleotides, wherein removing the hybrid region includes contacting the second hybrid region with an RNase enzyme, such as an RNase H enzyme.

In other forms, the hybrid region includes one or more inosine bases, and wherein removing the hybrid region includes contacting the second hybrid region with an endonuclease enzyme that cleaves the one or more inosine bases, such as an Endonuclease V enzyme (ENDOV).

In other forms, the hybrid region includes one or more unique target sequences, wherein the one or more unique target sequences are not present within the target DNA molecule, and wherein removing the hybrid region includes contacting the second hybrid region with a nickase enzyme that selectively cleaves or removes the one or more unique target sequences.

In other forms, the hybrid region includes one or more methylated GATC sequences, and wherein removing the second hybrid region includes contacting the second region with a methylation-specific nuclease, such as DpnI.

### E. Second Oligonucleotide Primer Design

Second oligonucleotide primer molecules and their use in the described methods are provided. The second primer typically includes one functional nucleic acid “domain”, including or entirely formed from removable nucleotides. In some forms, the composition and sequence of the second oligonucleotide primer is exactly the same as that of the removable hybrid region of a first oligonucleotide primer or an adapter molecule. In other forms, the composition and/or sequence of the second oligonucleotide primer are substantially the same as that of a removable hybrid region of the first oligonucleotide primer or an adapter molecule. In other forms, the composition of the second oligonucleotide primer is not the same as that of a removable hybrid region of a first oligonucleotide primer or an adapter molecule. However, in all cases, the second primer is configured to hybridize to the 5′ “sticky end” region of ssDNA of a truncated dsDNA molecule that is created upon removal of the removable hybrid region (e.g., following removal of the hybrid region of the first primer or the truncated dsDNA molecule).

The second primer typically includes from about 4 to about 100 nucleotides, inclusive, or any integer (or range of integers) of nucleotides in between the indicated values, for example, between about 8 to about 64 nucleotides, inclusive, or between about 6 to about 12 nucleotides, inclusive. In some forms, the second primer includes about 4, 5, 6, 7, 8, 9, 10, 11, 12, 13, 14, 15 or 16, or more nucleotides. In some forms, the second primer includes about 8 nucleotides.

In some forms, the second primer includes a single type of nucleic acid. For example, in some forms, the second primer includes RNA, or DNA. In other forms, the second primer includes two types of nucleic acid, such as DNA and inosine, or DNA and methylated DNA. In other forms, the second primer includes a DNA nucleotide sequence that functions as an enzyme recognition sequence, such as a restriction endonuclease recognition sequence. In an exemplary form, the composition of the second primer is the same as that of the second hybrid region of a first primer or an adapter. Therefore, in some forms, the same removing agent can be used to remove the removable hybrid region of the first primer or an adapter and the second primer. In other forms, the composition of the second primer is not the same as that of the second hybrid region of the first primer. Therefore, in some forms, the same removing agent cannot be used to remove the second hybrid region and the second primer, but a different removing agent should be applied to achieve removal of each domain, in turn. For example, in some forms, the second hybrid domain includes a DNA nucleotide sequence that includes a RE cut site, and an RE is required to remove the second hybrid domain, whereas the second primer includes a corresponding sequence of RNA nucleotides, and an RNase is required to remove the second primer.

In some forms, the second oligonucleotide primer includes RNA nucleotides that are selectively and specifically digested upon exposure to Ribonucleases (RNase) enzymes. An exemplary RNase enzyme suitable for use in the described methods includes RNaseH. Therefore, in some forms, the second primer includes RNA bases that are selectively removable upon exposure to an RNaseH enzyme.

In other forms, the second oligonucleotide primer includes inosine nucleotides that are selectively and specifically digested upon exposure to endonucleases enzymes. Therefore, in some forms, the second primer includes inosine bases that are selectively removable upon exposure to an endonuclease V enzyme.

In other forms, the second oligonucleotide primer includes a DNA nucleotide sequence that is selectively and specifically digested upon exposure to one or more restriction endonucleases (RE) enzymes. Therefore, in some forms, the second primer includes a DNA nucleotide sequences that is selectively cleaved upon exposure to an RE enzyme, such as a nicking endonuclease.

In other forms, the second oligonucleotide primer includes methylated nucleotide sequences that are selectively and specifically digested upon exposure to DpnI enzymes. Therefore, in some forms, the second primer includes a methylated guanine, for example, a G^{methyl} ATC nucleotide sequence, that is selectively removable upon exposure to a DpnI enzyme.

### F. Strand Displacing Polymerases

Strand displacing polymerases (SDP) for use with the described methods are also provided. Any suitable SDP enzymes can be used in the described methods. Exemplary SDPs include Bst2.0, Bst3.0, Bsu and Phil29. In particular forms, the SDP is Bst2.0 or Bst3.0.

Typically, the extension of a first or second primer is initiated by a SDP enzyme that initiates an amplification from the second primer using a single strand of a target nucleic acid as a template, to yield a new (i.e., “third”) nucleic acid strand that displaces the entire first strand of the hybridized second molecule. The extension thereby provides (i) a ds nucleic acid including the target, and (ii) a displaced single stranded nucleic acid that is or includes the entire second strand of the target ds nucleic acid (see, e.g., 12920 in FIG. 12D, which provides 12900 and 12930, or 1340 in FIG. 13G, which provides 13010 and 1350). In some forms, the displaced ssDNA molecule includes a sequence complementary to the target DNA molecule, or a sequence that is a copy of a single strand of the target nucleic acid. The displaced ssDNA molecules may be collected, pooled, and subject to downstream processes such as library preparation and/or sequencing.

### G. One-Step Methodologies Including Pre-Formed Reaction Mixtures

Any of the described step-wise methods for amplification of a target nucleic acid can be carried out by contacting a target nucleic acid with a reaction mixture that includes two or more of the reagents necessary for two or more of the described steps. For example, in some forms, steps (a), (b), (c), (d), (c) and/or (f) can be carried out following contacting the target nucleic acid with a reaction mixture that includes reagents necessary for steps (a), (b), (c), (d), (c) and/or (f).

In an exemplary method, a target nucleic acid is contacted with a reaction mixture that includes one or more of a strand displacing polymerase (SDP), dNTPs, a first primer, a second primer, a removal agent, and buffers suitable for one or more of the steps (a), (b), (c), (d), (c) and/or (f). For example, in an exemplary method, one or more of the steps of hybridizing a first primer to a first primer binding site in a target DNA molecule, extending the first primer from its 3′ end using the target DNA molecule as a template, removing the second hybrid region from the extended hybrid dsDNA molecule to form a truncated dsDNA molecule comprising a region of ssDNA, hybridizing a second primer to the region of ssDNA of the truncated dsDNA molecule to form a hybridized second molecule, and extending the hybridized second primer of the hybridized second molecule using a strand-displacing DNA polymerase are carried out following a single step of contacting a target nucleic acid molecule with a reaction mixture including one or more of a first primer, a second primer; an enzyme specific for the hybrid region; and a strand displacing DNA polymerase, and dNTPs/buffers required for the enzymic reagents.

### H. Additional Method Steps/Integrated Methodologies

Any of the described methods for amplification of a target nucleic acid can include one or more additional steps or encompass a range of targets and employ a range of reagents, as required by the user or assay.

In some forms, the described methods for amplification of a target nucleic acid are integrated within, or performed as an additional component of one or more methods for spatial analysis of analytes in a biological sample, e.g., a sample from which a target nucleic acid is produced. In some forms, the methods increase the amount of a target nucleic acid that is isolated and/or identified within a biological sample. For example, in some forms, the methods are implemented within the workflow of a spatial analysis protocol, for example, subsequent to capturing a target analyte on a capture probe and extending the captured probe to provide a target nucleic acid for amplification according to the described methods. Typically, the target nucleic acid is conjugated to a substrate, such as an array, prior to the described methods for amplification.

In some forms, methods for spatial analysis require that a biological sample be disposed within or on a substrate, such as a slide or other matrix. Therefore, in some forms, methods including one or more of steps (a)-(f) for amplification of a target nucleic acid (described above) further include, prior to step (a), providing the target nucleic acid disposed on a first substrate. For example, in some forms, one or more additional steps and/or reagents are incorporated into or carried out before, during or after any one of the steps (a), (b), (c), (d), (c) or (f) of any one of the described methods.

In some forms, when the additional steps include or require providing, creating, modifying or otherwise effecting a target nucleic acid for amplification according to the described methods, the additional steps are typically carried out prior to step (a). In other forms, where the additional steps relate to one or more intermediate steps that include a pre-formed target nucleic acid, the methods can include one or more additional steps before, during or after any one of steps (a), (b), (c), (d), (c) or (f). In other forms, where the additional steps process or otherwise require the product, such as the amplified target nucleic acid or a complement thereof, the methods are typically carried out after step (d), or (e), or (f), depending upon when the product is produced according to the methods.

### 1. Supplemental Spatial Analysis Workflows

In some forms, the methods for spatial analysis implement the same or modified protocol for amplifying a target nucleic acid after a biological sample, from which the target nucleic acid is produced and/or isolated, is subjected to spatial analysis methodologies, such that the methods are supplemental to existing spatial analysis workflows. For example, in some forms, when a target analyte within a sample is an RNA, and when the spatial analysis methods include use of a first oligonucleotide probe and optionally a second oligonucleotide probe (e.g., first and second RTL probes), the methods can include, prior to step (a), one or more steps of hybridizing a first probe and optionally a second probe to one or more RNA analytes present within the sample. In some forms, according to spatial analysis workflows, the first probe and optionally the second probe each include a sequence that is substantially complementary to a sequence present in a target RNA analyte. In some forms, according to spatial analysis workflows, one or more of a first probe or second probe includes a capture probe binding domain. Therefore, in some forms, a first probe and optionally a second probe each include a sequence that is substantially complementary to a target RNA analyte within the biological sample, and whereby the first probe and/or second probe include a capture probe binding domain.

In some forms, when the methods include a first RTL probe and a second RTL probe, the methods further include one or more steps of coupling the first probe and the second probe subsequent to their hybridization to a target nucleic acid analyte, thereby generating a connected probe. Typically, a connected probe includes a single capture probe binding domain (e.g., derived from the first probe or second probe). The “capture probe binding domain” is a domain including a sequence that is complementary to a particular capture domain present in a capture probe on an array. In some forms, the capture probe binding domain includes a poly(A) sequence. In some forms, the capture probe binding domain includes a poly-uridine sequence, a poly-thymidine sequence, or a combination thereof. In some forms, the capture probe binding domain includes a random sequence (e.g., a random hexamer or octamer). In some forms, the capture probe binding domain is complementary to a capture domain in a capture probe. In some instances, the capture probe binding domain or a complement thereof is on 5′ end of the first probe or second probe. In some instances, the capture probe binding domain or a complement thereof is on 3′ end of one of the first probe or second probe.

In some forms, after hybridization of RTL probes (e.g., first RTL and the second RTL probes) to the target analyte, the RTL probes (e.g., the first RTL probe and the second RTL probe) are coupled (e.g., ligated) together, creating a single connected probe (e.g., a ligation product) that is complementary to the target analyte. Ligation can be performed enzymatically or chemically. For example, the first and second RTL probes are hybridized to the first and second target regions of the analyte, and the first and second RTL probes are subjected to a nucleic acid reaction to ligate them together.

The connected probe (e.g., ligation product) that results from the coupling (e.g., ligation) of the first RTL probe and second RTL probe can serve as a proxy for the target analyte, as such an mRNA. In some forms, the methods include providing a plurality of first probes and a plurality of second probes, wherein a pair of probes for a target analyte includes both a first and second probe (e.g., a first and second RTL probe). Further, it is appreciated that probe pairs can be designed to cover any gene of interest within a biological sample. For example, a pair of probes can be designed so that each analyte, e.g., a whole exome, a transcriptome, a genome, can conceivably be detected using a probe pair. In some instances, probe pairs are designed to cover an entire transcriptome of a species (e.g., a mouse or a human). In some instances, probes are designed to cover a subset of a transcriptome (e.g., a mouse or a human). In some instances, the methods disclosed herein utilize about 1000, about 2000, about 3000, about 4000, about 5000, about 6000, about 7000, about 8000, about 9000, about 10,000, about 15,000, about 20,000, or more probe pairs.

In some forms, when the methods further include providing the biological sample disposed on a first substrate, the methods include one or more steps of aligning the first substrate with a second substrate including an array, such that at least a portion of the biological sample is aligned with at least a portion of the array. Typically, an array for use with methods for spatial analyses includes a plurality of capture probes. In some forms, a capture probe of the plurality of capture probes includes a spatial barcode and a capture domain, e.g., for capture of a capture domain binding domain of a target analyte-binding probe, a target nucleic acid analyte (e.g., mRNA) or a connected probe or ligation product.

In some forms, the methods further include one or more steps of releasing the connected probe (or ligation product) from the nucleic acid (e.g., RNA) analyte when at least a portion of the biological sample is aligned with at least a portion of the array to provide a released probe. Methods for releasing a nucleic acid component, including connected probe(s), from a biological sample typically include contacting the biological sample with one or more reagents for digesting or otherwise removing protein, lipid, carbohydrate and/or and other non-nucleic acid components from the biological sample. In some forms, to release the connected probe (e.g., a ligation product), an endoribonuclease (e.g., RNase A, RNase C, RNase H, or RNase I) is used. An endoribonuclease such as RNase H specifically cleaves RNA in RNA: DNA hybrids. In some forms, the connected probe (e.g., a ligation product) is released enzymatically. In some forms, the endoribonuclease is an RNase H. In some forms, the RNase H is RNase H1 or RNase H2. In some forms, releasing the first probe or the connected probe from the RNA analyte includes contacting the sample with an RNase, e.g., RNase H.

In some forms, according to a spatial analysis methodology, the methods further include one or more steps of hybridizing the released connected probe (or other analyte such as an analyte capture agent derived oligonucleotide) to the capture domain of the capture probe. In some forms, the methods further include one or more steps for isolating, quantifying and/or characterizing spatial information for one or more captured analytes.

In some forms, the connected probe (or other analyte such as an analyte capture agent derived oligonucleotide) is extended using the capture probe as a template and/or the capture probe is extended using the connected probe (or other analyte such as an analyte capture agent derived oligonucleotide) as a template. In some forms, a capture probe bound to a connected probe (“captured probe”) or other analyte such as an analyte capture agent derived oligonucleotide can be extended (thereby generating an “extended captured probe,” e.g., as described herein). Therefore, in some forms, the methods include extending a capture probe bound to a connected probe (or other analyte such as an analyte capture agent derived oligonucleotide) to provide an extended captured probe. For example, extending a capture probe bound to a connected probe can include generating cDNA from a captured (hybridized) nucleic acid hybridized to the complementary region of the capture probe.

An exemplary captured nucleic acid is an RNA, such as an mRNA. This process typically involves synthesis of a complementary strand of the hybridized nucleic acid, e.g., generating cDNA based on the captured RNA template (the RNA hybridized to the capture domain of the capture probe). Thus, in an initial step of extending a captured probe, e.g., the cDNA generation, the captured (hybridized) nucleic acid, e.g., RNA, acts as a template for the extension, e.g., reverse transcription, step.

In some forms, reverse transcription (RT) reagents can be added to permeabilized biological samples. Incubation with the RT reagents can produce spatially-barcoded full- or partial-length cDNA from the captured analytes (e.g., polyadenylated mRNA). Second strand reagents (e.g., second strand primers, enzymes) can be added to the biological sample to initiate second strand synthesis.

In some forms, the extension includes reverse transcription. For example, reverse transcription includes synthesizing cDNA (complementary or copy DNA) from RNA, e.g., (messenger RNA), using a reverse transcriptase. In some forms, reverse transcription is performed while the biological sample is still in place, generating an analyte library, where the analyte library includes the spatial barcodes from the associated capture probes. In some forms, a captured probe is extended using one or more DNA polymerases. In some forms, a capture probe binding domain of a first probe, or a second probe, and/or a capture domain of a capture probe includes a primer for producing the complementary strand of a nucleic acid hybridized to the probe, e.g., a primer for DNA polymerase and/or reverse transcription. The nucleic acid, e.g., DNA and/or cDNA, molecules generated by the extension reaction incorporate the sequence of the first probe, second probe or capture probe, respectively. The extension of the captured probe, e.g., a DNA polymerase and/or reverse transcription reaction, can be performed using a variety of suitable enzymes and protocols.

Thus, in some forms, the target nucleic acid subject to the disclosed amplification methods is an extended capture probe including cDNA corresponding to all or portion of a target nucleic acid analyte from a biological sample.

In some forms, a full-length DNA (e.g., cDNA) molecule is generated. In some forms, a “full-length” DNA molecule refers to the whole of a “captured” nucleic acid molecule. However, if a nucleic acid (e.g., RNA) was partially degraded in the tissue sample, then the captured nucleic acid molecules will not be the same length as the initial RNA in the tissue sample. In some forms, 3′ end of an extended captured probe, e.g., first strand cDNA molecules, is modified. For example, a linker or adaptor can be ligated to the 3′ end of the extended captured probes. This can be achieved using single stranded ligation enzymes such as T4 RNA ligase or CIRCLIGASE™ (available from Lucigen, Middleton, WI). In some forms, template switching oligonucleotides are used to extend cDNA in order to generate a full-length cDNA (or as close to a full-length cDNA as possible). In some forms, a second strand synthesis helper probe (a partially double stranded DNA molecule capable of hybridizing to the 3′ end of the extended captured probe), can be ligated to the 3′ end of the extended captured probe, e.g., first strand cDNA, molecule using a double stranded ligation enzyme such as T4 DNA ligase. Other enzymes appropriate for the ligation step are known in the art and include, e.g., Tth DNA ligase, Taq DNA ligase, Thermococcus sp. (strain 9°N) DNA ligase (9° N™ DNA ligase, New England Biolabs), AMPLIGASE™ (available from Lucigen, Middleton, WI), and SPLINTR™ (available from New England Biolabs, Ipswich, MA). In some forms, a polynucleotide tail, e.g., a poly(A) tail, is incorporated at the 3′ end of the extended captured probe/cDNA molecules. In some forms, the polynucleotide tail is incorporated using a terminal transferase active enzyme.

Any of the described additional steps for spatial analysis can be incorporated before, during, or after step (a) of the described methods for amplifying a target nucleic acid.

### 2. Ligation

In some forms, linking to a 3′ end of the first DNA strand of the dsDNA molecule a nucleic acid adapter includes one or more steps to ligate the nucleic acid adapter to the dsDNA molecule. For example, in some forms, a component of an adapter, such as a second adapter strand, includes a 5′ phosphate moiety, and 5′ end of a second adapter strand is blunt end ligated to the 3′ end of a second DNA strand of a target dsDNA molecule. In some forms, the ligation includes enzymatic ligation. using a double stranded ligation enzyme such as T4 DNA ligase. Other enzymes appropriate for the ligation step are known in the art and include, e.g., Tth DNA ligase, Taq DNA ligase, Thermococcus sp. (strain 9°N) DNA ligase (9° N™ DNA ligase, New England Biolabs), AMPLIGASE™ (available from Lucigen, Middleton, WI), and SPLINTR® (available from New England Biolabs, Ipswich, MA). For example, any of the first or second oligonucleotide primers or adapter molecules may be subjected to an enzymatic ligation reaction using a ligase (e.g., T4 RNA ligase (Rnl2), a SPLINTR® ligase, or a T4 DNA ligase). See, e.g., Zhang L., et al.; Archaeal RNA ligase from Thermoccocus kodakarensis for template dependent ligation RNA Biol. 2017; 14(1): 36-44 for a description of KOD ligase. A skilled artisan will understand that various reagents, buffers, cofactors, etc. may be included in a ligation reaction depending on the ligase being used.

### 3. Template Switching Oligonucleotide Workflows

In some forms, prior to step (a), the methods include forming a target DNA molecule that includes ssDNA generated from a template-switching workflow. In some forms, template switching oligonucleotides are used to extend cDNA in order to generate a full-length cDNA (or as close to a full-length cDNA as possible). In some forms, a second strand synthesis helper probe (a partially double stranded DNA molecule capable of hybridizing to the 3′ end of the extended captured probe), can be ligated to the 3′ end of the extended captured probe, e.g., first strand cDNA, molecule using a double stranded ligation. In some forms, a polynucleotide tail, e.g., a poly(A) tail, is incorporated at the 3′ end of the extended captured probe/cDNA molecules. In some forms, the polynucleotide tail is incorporated using a terminal transferase active enzyme. In some forms, the captured analytes can be spatially-barcoded by performing a reverse transcriptase first strand cDNA reaction using template switching oligonucleotides.

A “template switching oligonucleotide” is an oligonucleotide that hybridizes to untemplated nucleotides added by a reverse transcriptase (e.g., enzyme with terminal transferase activity) during reverse transcription. In some forms, a template switching oligonucleotide hybridizes to untemplated poly(C) nucleotides added by a reverse transcriptase. In some forms, the template switching oligonucleotide adds a common 5′ sequence to cDNA that can be used for cDNA amplification.

For example, a template switching oligonucleotide (TSO) can hybridize to a poly(C) tail added to a 3′end of the cDNA by a reverse transcriptase enzyme in a template-independent manner. The hybridized TSO is used to further extend the first strand cDNA such that it includes a complement of the TSO. The original nucleic acid template (e.g., mRNA) and template switching oligonucleotide can then be denatured from the extended cDNA and the spatially-barcoded capture probe can then hybridize with the cDNA and a complement of the cDNA (i.e., second strand) can be generated. In some forms, the TSO (or a primer having a similar sequence thereto) can be used to prime synthesis of a second strand cDNA templated from the first strand cDNA. The first strand cDNA can then be purified and collected for downstream amplification steps. The first strand cDNA can be amplified using in accordance with the amplification methods disclosed herein, generating a library associated with a particular spatial barcode. In some forms, the library preparation can be quantitated and/or quality controlled to verify the success of the library preparation steps.

In some forms, the template switching oligonucleotide adds a common sequence onto the 5′ end of an RNA being reverse transcribed. For example, a template switching oligonucleotide can hybridize to untemplated poly(C) nucleotides added onto the end of a cDNA molecule and provide a template for the reverse transcriptase to continue replication to the 5′ end of the template switching oligonucleotide, thereby generating full length cDNA ready for further amplification.

In some forms, once a cDNA molecule is generated, the template switching oligonucleotide can serve as a primer in a cDNA amplification reaction. In some forms, a template switching oligonucleotide is added before, contemporaneously with, or after a reverse transcription, or other terminal transferase-based reaction. In some forms, a template switching oligonucleotide is included in the capture probe. In certain forms, methods of sample analysis using template switching oligonucleotides can involve the generation of nucleic acid products from analytes of the biological sample, followed by further processing of the nucleic acid products with the template switching oligonucleotide.

Template switching oligonucleotides can include a hybridization region and a template region. The hybridization region can include any sequence capable of hybridizing to the target. In some forms, the hybridization region can, e.g., include a series of G bases to complement the overhanging C bases at the 3′ end of a cDNA molecule. The series of G bases can include 1 G base, 2 G bases, 3 G bases, 4 G bases, 5 G bases, or more than 5 G bases. The template sequence can include any sequence to be incorporated into the cDNA.

In other forms, the hybridization region can include at least one base in addition to at least one G base. In other forms, the hybridization can include bases that are not a G base. In some forms, the template region includes at least 1 (e.g., at least 2, 3, 4, 5 or 10 more) tag sequences and/or functional sequences. In some forms, the template region and hybridization region are separated by a spacer.

In some forms, the template regions include a barcode sequence. The barcode sequence can act as a spatial barcode and/or as a unique molecular identifier. Template switching oligonucleotides can include deoxyribonucleic acids; ribonucleic acids; modified nucleic acids including 2-aminopurine, 2,6-diaminopurine (2-amino-dA), inverted dT, 5-methyl dC, 2′-deoxyinosine, Super T (5-hydroxybutynl-2′-deoxyuridine), Super G (8-aza-7-deazaguanosine), locked nucleic acids (LNAs), unlocked nucleic acids (UNAs, e.g., UNA-A,
UNA-U, UNA-C, UNA-G), Iso-dG, Iso-dC, 2′ fluoro bases (e.g., Fluoro C, Fluoro U, Fluoro A, and Fluoro G), or any combination of the foregoing.

In some forms, the length of a template switching oligonucleotide can be at least about 1, 2, 10, 20, 50, 75, 100, 150, 200, or 250 nucleotides or longer. In some forms, the length of a template switching oligonucleotide can be at most about 2, 10, 20, 50, 100, 150, 200, or 250 nucleotides or longer.

In an exemplary form, a template switching oligonucleotide includes a second primer binding sequence, and a generated target DNA molecule further includes the second primer binding sequence or a complement thereof.

An exemplary method for amplification of a target DNA molecule from a template-switching workflow includes:
(a) forming a target ssDNA molecule from a template-switching workflow, including attaching a template switching oligonucleotide to a captured probe to form the target nucleic acid,
wherein the template switching oligonucleotide further includes a second primer binding sequence, and
wherein the generated target ssDNA molecule further includes the second primer binding sequence or a complement thereof;
(b) hybridizing a second primer to the second primer binding sequence of the target ssDNA molecule to form a hybridized second molecule including a hybridized second primer,
wherein the second primer includes a second hybrid region including one or more of RNA nucleotides, DNA nucleotides including a unique target sequence, or inosine nucleotides; and
(c) extending the hybridized second primer of the hybridized second molecule using a strand-displacing DNA polymerase to provide an extended hybrid double-stranded (ds) DNA molecule;
(d) removing the second primer from the extended hybrid double-stranded (ds) DNA molecule to form a truncated dsDNA molecule;
(e) hybridizing a second primer to the second primer binding sequence of the truncated dsDNA molecule to form a hybridized second molecule including a hybridized second primer; and
(f) extending the hybridized second primer of the hybridized second molecule using a strand-displacing DNA polymerase to provide an extended hybrid double-stranded (ds) DNA molecule and a displaced single-stranded (ss) DNA molecule.

### 4. Blocked Probe Workflows

In some forms, prior to step (a), the methods include forming a blocked target DNA molecule that includes ssDNA from a probe-blocking workflow. For example, in some forms, the methods include blocking additional free 3′ ends of a target nucleic. Exemplary blocking method include, immediately prior to step (a), contacting the target nucleic acid with an exonuclease enzyme to remove any un-extended oligos with free 3′ ends from array. In some forms, the methods include. In other forms, the methods include, immediately prior to step (a), modifying the free 3′ ends on the array by the addition of inverted dT or inverted dideoxy dT. In other forms, the methods include, immediately prior to step (a), contacting the array with a plurality of poly-A oligonucleotides, whereby the plurality of poly-A oligonucleotides bind to free poly T in the array.

In some forms, double-stranded extended captured probes are treated to remove any un-extended captured probes prior to amplification and/or analysis, e.g., sequence analysis. This can be achieved by a variety of methods, e.g., using an enzyme to degrade the un-extended captured probes, such as an exonuclease enzyme, or purification columns.

### 5. Sample Preparation Workflows

In some forms the methods include, prior to step (a), contacting a biological sample with one or more permeabilizing reagents, wherein the biological sample includes a target nucleic acid analyte. Exemplary permeabilizing reagents are selected from an organic solvent, a detergent, and an enzyme, or a combination thereof. In some forms, the permeabilization agent is selected from the group consisting of an endopeptidase, a protease, sodium dodecyl sulfate (SDS), polyethylene glycol tert-octylphenyl ether, polysorbate 80, polysorbate 20, N-lauroylsarcosine sodium salt solution, saponin, Octoxynol-9; t-octylphenoxypolyethoxyethanol and polysorbate-20. An exemplary permeabilization agent includes a protease. In some form, the method further includes, after contacting the biological sample with one or more permeabilizing reagents, removing the one or more permeabilizing reagents. For example, in some forms, removing the one or more permeabilizing reagents includes contacting the biological sample with a wash solution.

In other forms, the methods further include, prior to step (a), contacting the biological sample with one or more releasing reagents. In some forms, the one or more releasing reagents remove a nucleic acid analyte hybridized to the target DNA molecule. An exemplary releasing reagent includes a nuclease. In an exemplary form, the analyte is an RNA molecule, and wherein the nuclease includes an RNase, preferably RNase H.

In some forms, the methods further include, before or after step (a), one or more steps for imaging a biological sample including a target nucleic acid. In some forms, a biological sample includes a tissue sample. Exemplary tissue samples include a fixed sample, optionally a Formalin-Fixed Paraffin-Embedded (FFPE) sample. Therefore, in some forms, the methods include, prior to step (a) one or more steps of de-crosslinking the tissue sample. In other forms, the tissue sample is or includes a fresh, frozen sample. When the sample includes a FFPE sample, the methods can further include, prior to step (a), deparaffinizing the tissue sample. Typically, deparaffinizing includes contacting the tissue sample with a solvent.

In some forms, the methods further include, before (a), one or more steps of staining and/or labelling the biological sample. In some forms, staining the tissue sample includes hematoxylin and/or cosin (H and E) staining. In some forms, the methods further include imaging the stained and/or labelled tissue sample. In some forms, the methods further include de-staining the tissue sample.

In some forms, the methods include one or more steps to de-paraffinize a biological sample that includes paraffin wax. For example, in some forms, the biological is treated with a series of washes that include xylene and various concentrations of ethanol. In some forms, methods of deparaffinization include treatment of xylene (e.g., three washes at 5 minutes each). In some forms, the methods further include treatment with ethanol (e.g., 100% ethanol, two washes 10 minutes each; 95% ethanol, two washes 20 minutes each; 70% ethanol, two washes 10 minutes each; 50% ethanol, two washes 10 minutes each). In some forms, after ethanol washes, the biological sample is washed with deionized water (e.g., two washes for 5 minutes each). In some embodiments, the biological sample (e.g., FFPE sample) is permeable after deparaffinization. In some embodiments, processing of the biological sample, such as de-waxing, allows the biological sample to become permeabilized. It is appreciated that one skilled in the art can adjust these methods to optimize deparaffinization.

In some forms, the biological sample is de-crosslinked. For example, in some forms, the biological sample or blocked sample is de-crosslinked in a solution containing TE buffer (including Tris and EDTA). In some forms, the TE buffer is basic (e.g., at a pH of about 9). In some forms, de-crosslinking occurs at about 50° C. to about 80° C. In some forms, de-crosslinking occurs at about 70° C. In some forms, de-crosslinking occurs for about 1 hour at 70° C. For example, in some forms, just prior to de-crosslinking, the biological sample or blocked sample is treated with an acid (e.g., 0.1 M HCl for about 1 minute). After the decrosslinking step, the biological sample or blocked sample can be washed (e.g., with 1×PBST). In some forms, the biological sample is permeabilized using a phosphate buffer. In some forms, the phosphate buffer is PBS (e.g., 1×PBS). In some forms, the phosphate buffer is PBST (e.g., 1×PBST). In some forms, the permeabilization step is performed multiple times (e.g., 3 times at 5 minutes each).

In some forms, permeabilization occurs using a protease. In some forms, the protease is an endopeptidase. Endopeptidases that can be used include but are not limited to trypsin, chymotrypsin, elastase, thermolysin, pepsin, clostripan, glutamyl endopeptidase (GluC), ArgC, peptidyl-asp endopeptidase (ApsN), endopeptidase LysC and endopeptidase LysN. In some forms, the endopeptidase is pepsin. In some forms, the protease is proteinase K.

In some forms, after creating a connected probe (e.g., by ligating a first probe and/or a second probe that are hybridized to adjacent sequences in a target RNA or ligating a circularizable probe such as a padlock probe), the biological sample is permeabilized. In some forms, the biological sample is permeabilized contemporaneously with, or prior to, contacting the biological sample with a first probe and/or a second probe, e.g., hybridizing and coupling the first probe and the second probe to the provide a combined probe, and then permeabilizing the biological sample to release the connected probe from the analytes in the sample.

In some forms, methods provided herein include permeabilization of the biological sample such that a probe/bound analyte can more easily hybridize to the immobilized capture probe (e.g., compared to no permeabilization).

In some forms, the permeabilization step includes application of a permeabilization buffer to the biological sample. In some forms, the permeabilization buffer includes a buffer (e.g., Tris pH 7.5), MgCl2, a detergent (e.g., sarkosyl detergent, also known as sodium lauroyl sarcosinate), enzyme (e.g., proteinase K), and nuclease free water. In some forms, the permeabilization step is performed at 37° C. In some forms, the permeabilization step is performed for about 20 minutes to 2 hours (e.g., about 20 minutes, about 30 minutes, about 40 minutes, about 50 minutes, about 1 hour, about 1.5 hours, or about 2 hours). In some forms, the releasing step is performed for about 40 minutes.

In some forms, after generating a connected probe/ligation product, the connected probe/ligation product is released from the analyte. In some forms, a connected probe/ligation product is released from the analyte using an endoribonuclease. In some forms, the endoribonuclease is RNase H, RNase A, RNase C, or RNase I. In some forms, the endoribonuclease is RNase H. RNase H is an endoribonuclease that specifically hydrolyzes the phosphodiester bonds of RNA, when hybridized to DNA.

RNase H is part of a conserved family of ribonucleases which are present in many different organisms. There are two primary classes of RNase H: RNase H1 and RNase H2. Retroviral RNase H enzymes are similar to the prokaryotic RNase H1. All of these enzymes share the characteristic that they are able to cleave the RNA component of an RNA: DNA heteroduplex. In some forms, the RNase His RNase H1, RNase H2, or RNase H1, or RNase H2. In some forms, the RNase H includes but is not limited to RNase HII from Pyrococcus furiosus, RNase HII from Pyrococcus horikoshi, RNase HI from Thermococcus litoralis, RNase HI from Thermus thermophilus, RNase HI from E. coli, or RNase HII from E. coli. In some forms, the releasing step is performed using a releasing buffer. In some forms, the release buffer includes one or more of a buffer (e.g., Tris pH 7.5), enzyme (e.g., RNase H) and nuclease-free water.

In some forms, the releasing step is performed at 37° C. In some forms, the releasing step is performed for about 20 minutes to 2 hours (e.g., about 20 minutes, about 30 minutes, about 40 minutes, about 50 minutes, about 1 hour, about 1.5 hours, or about 2 hours). In some forms, the releasing step is performed for about 30 mins. In some forms, the releasing step occurs before the permeabilization step. In some forms, the releasing step occurs after the permeabilization step. In some forms, the releasing step occurs at the same time as the permeabilization step (e.g., in the same buffer).

### 6. Sequence Determination

Any of the described methods for amplifying a target DNA molecule provided herein can include one or more steps for sequence analysis of the amplified DNA molecules. In some aspects, the provided methods involve analyzing, e.g., detecting or determining, one or more sequences present in a target nucleic acid, for example, by sequence analysis of a replicated nucleic acid. In some forms, the described methods further include one or more steps for step of determining a sequence of a target nucleic acid (including determining the sequence of an amplified or displaced strand of a target nucleic acid, as described herein).

In some aspects, provided herein are methods comprising in situ assays using microscopy as a readout for sequence determination, e.g., nucleic acid sequencing, hybridization, or other detection or determination methods involving an optical readout.

In some forms, a step of determining is carried out after the conclusion of one or more of the described methods for amplifying a target nucleic acid to produce an amplified product. In some forms, a step of determining a sequence includes a further step of amplifying a target nucleic acid to produce a further amplified product.

An exemplary amplified target nucleic acid includes (i) all or part of a sequence of a connected probe/ligation product bound to a capture domain, or a complement thereof, and (ii) a sequence of a spatial barcode, or a complement thereof. For example, in some forms, the determining step includes sequencing to determine (i) all or part of a sequence of the connected probe/ligation product bound to the capture domain, or a complement thereof, and (ii) the spatial barcode, or a complement thereof. In some forms, the sequencing step includes in situ sequencing, Sanger sequencing, next-generation sequencing, and/or nanopore sequencing.

In some forms, the methods further include subjecting a region of interest in the biological sample to spatial transcriptomic analysis. In some forms, a target nucleic acid, or amplification product thereof includes a unique molecular identifier (UMI). In some forms, a target nucleic acid includes a cleavage domain. In some forms, the cleavage domain includes a sequence recognized and cleaved by a uracil-DNA glycosylase, apurinic/apyrimidinic (AP) endonuclease (APEI), U uracil-specific excision reagent (USER), and/or an endonuclease VIII. In some forms, a target nucleic acid does not include a cleavage domain and therefore, for example, is not cleaved from an array.

### III. Kits and Compositions for Methods

Provided herein are kits that include one or more compositions and/or reagents for performing the described methods of amplifying a target nucleic acid, for example, within of following a spatial analysis workflow.

In some aspects, provided herein are compositions comprising any of the reagents for performing extension by a strand displacing polymerase (SDP) using a ssDNA region as template to amplify a target nucleic acid, and oligonucleotide primers and/or adapter molecules (e.g., double stranded adapter molecules or partially double stranded adapter molecules), and/or intermediate molecules described herein.

Also provided herein are kits, for amplifying a target nucleic acid in a biological sample according to any of the methods described herein. In some embodiments, provided herein is a kit including any of the nucleic acid oligonucleotides described herein (e.g., first and/or second primers for extension using ssDNA as template). In some embodiments, the kit further includes any of the reagents, oligonucleotides, arrays, enzymes or nucleic acids disclosed herein. In some embodiments, the kit includes a removable region-specific enzyme, e.g., an endonuclease such as RNaseH.

The various components of the kit may be present in separate containers or certain compatible components may be pre-combined into a single container. In some embodiments, the kits further contain instructions for using the components of the kit to practice the provided methods.

A primer set for use with the described methods is provided. Typically, the primer set includes:
(a) a first primer, including
   (i) a first region including DNA bases complementary to a first primer binding site of a target nucleic acid; and
   (ii) a second hybrid region,
   wherein the second region includes one or more of RNA bases, DNA bases including a unique target sequence, inosine bases, or a methylated GATC sequence; and(s)
(b) a second primer, including the second hybrid region of the first primer.

In some forms, the second primer is formed entirely of the second hybrid region of the first primer. An exemplary first primer is between about 8 and about 100 bases, inclusive. In some forms, the first primer includes a first region including between about four and about thirty bases, inclusive. In other forms, the first primer includes a second region including between about four and about thirty bases, inclusive. In some forms, the first region of the first primer includes one or more locked nucleic acids (LNA). In some forms, the second region of the first primer includes RNA nucleotides. In other forms, the second region of the first primer includes inosine nucleotides. In certain forms, the second region of the first primer includes a unique target sequence, for example, a target sequence including a nickase recognition sequence. In other forms, the second region of the first primer includes a methylated GATC sequence.

Specific kits including specific reagents for performing the described methods are also provided. In some forms, a kit includes one or more of:
(a) a first primer, including
   (i) a first region including DNA bases complementary to a first primer binding site of a target nucleic acid; and
   (ii) a second hybrid region,
   whereby the second region includes one or more of RNA bases, DNA bases including a unique target sequence, inosine bases, or a methylated GATC sequence; and
(b) instructions for performing the described methods for amplifying a target nucleic acid.

In some forms, the second region of the first primer includes RNA bases. In some forms, the kit further includes a second primer including the second region of the first primer. In some forms, the kit further includes an RNA-specific nuclease enzyme, such as an RNase enzyme, such as RNaseH.

In some forms, the second region of the first primer includes inosine nucleotides. In some forms, the kit includes a second primer including the inosine nucleotides of the second region of the first primer. In some forms, the kit further including an endonuclease enzyme that degrades the inosine nucleotides. An exemplary endonuclease enzyme includes Endonuclease V.

In some forms, the second region of the first primer includes one or more unique target sequences. In some forms, the kit further includes a second primer including the unique target sequences region of the first primer. In some forms, the kit further includes a nickase enzyme that selectively cleaves or removes the unique target sequences.

In some forms, the second region of the first primer includes one or more methylated GATC sequences. In some forms, the kit further includes a second primer including the one or more methylated GATC sequences of the first primer. In some forms, the kit further includes a methylation-specific nuclease enzyme. An exemplary methylation-specific nuclease enzyme includes DpnI.

In other forms, the kit includes a single stranded adapter or a double stranded adapter. An exemplary adapter includes nucleotides complementary to the second region of the first primer. In some forms, the first adapter includes a 5′ phosphate on the second strand.

In some forms, the kit further includes a ligase enzyme. An exemplary ligase enzyme includes a T4 ligase.

In some forms, the kit further includes a further including a strand displacing DNA polymerase, such as a Bst2.0, Bst3.0, Bsu or Phil29 strand displacing DNA polymerase.

In some forms, the kit further includes one or more of
(a) a solvent suitable to solubilize unbound first primer(s) and/or unbound second primer(s);
(b) one or more DNA-specific Polymerase enzymes;
(c) one or more nuclease enzymes; and/or
(d) buffers and/or reagents suitable for enzyme reactions according to the described methods for amplifying a nucleic acid.

### IV. Strand-Displacement Amplification of Target Nucleic Acids

Methods for amplification of a target nucleic acid (e.g., a captured nucleic acid analyte from a biological sample) containing single-stranded DNA (ssDNA) and double-stranded (dsDNA), have been developed. Compositions for use in the disclosed method are also expressly described herein.

In some forms, a target nucleic acid is amplified to yield quantities that are sufficient for analysis, e.g., via DNA sequencing. In some forms, a first strand of a target nucleic acid (e.g., DNA and/or cDNA molecules) acts as a template for the amplification reaction (e.g., an RNase mediated strand displacement reaction). In some forms, a second strand of a target nucleic acid (e.g., DNA and/or cDNA molecules) acts as a template for the amplification reaction (e.g., an RNase mediated strand displacement reaction).

In some forms, amplifying a target nucleic acid can function, e.g., to release copies of the target nucleic acid (amplification products or amplicons) from a substrate, such as a particle or bead.

In some forms, an amplified target nucleic acid or complement thereof is released, while the target nucleic acid itself remains attached to a substrate, such as a particle or bead.

In some embodiments, the methods provide a complementary (replicate) nucleic acid from a single-stranded target nucleic acid, a double stranded target nucleic acid, or a partially double stranded target nucleic acid. In some forms, the target nucleic acid is attached to a substrate, such as a particle or bead. An exemplary target nucleic acid is an extended capture probe, such as an extended capture probe conjugated to a substrate e.g., that is attached to a particle or bead. The methods employ strand displacing DNA polymerases to provide one or more copies of a nucleic acid having a nucleotide sequence that is the same or complementary to a target nucleic acid. Typically, the methods increase the amount of nucleic acids having a desired nucleotide sequence, or having the complement of a desired nucleotide sequence. In some forms, the methods increase the amount of a nucleic acid having the sequence of, or complementary to, a target nucleic acid by at least 100%, up to 1000%, or more than 1000%, such as up to 10,000% or up to 100,000% the original amount of the target nucleic acid prior to the methods. In some forms, the methods increase the amount of target nucleic acids in a mixture of target and non-target nucleic acids. Therefore, in some forms, the methods increase the ratio of target to non-target nucleic acids within a mixture of different nucleic acid species.

The target nucleic acid that is to be amplified according to the methods can be a single stranded (ss) nucleic acid, or can be one strand of a double stranded (ds) nucleic acid. In an exemplary form, the methods provide a multiplicity of copies of a single stranded nucleic acid having a nucleotide sequence that corresponds to the nucleotide sequence of a target nucleic acid attached to a substrate, such as a particle or bead. In some forms, the methods amplify a target nucleic acid at a constant temperature and/or upon addition of a single reaction mixture to a sample. In some forms, the methods provide one or more copies of a single stranded nucleic acid having a nucleotide sequence complementary to the nucleotide sequence of a target nucleic acid. In other forms, the methods provide one or more copies of a single stranded nucleic acid having a nucleotide sequence of a target nucleic acid. In some forms, the methods are carried out subsequent to one or more of the described methods for single cell analysis described herein.

The described methods for increasing the amount of a target nucleic acid within a sample are designed to be implemented within any molecular biological protocol, for example, to use in single cell nucleic acid expression experiments to increase the number of nucleic acids having a sequence corresponding to a captured analyte, such as a captured mRNA that is reverse transcribed into cDNA. Therefore, methods including amplification of a target nucleic acid including the nucleotide sequence of all or part of an analyte or proxy thereof (e.g., intermediate agent, ligation product) captured, for example, with a capture probe on a substrate, bead or partible, are also provided. In some forms, one or more of the described methods for amplifying a target nucleic acid are implemented within one or more steps of a single cell analysis protocol. Typically, one or more steps for amplifying a target nucleic acid are performed subsequent to capturing an analyte from a sample that is subject to a single cell assay. In some forms, the methods enhance the resolution, sensitivity, specificity and/or accuracy of single cell analysis of a target nucleic acid analyte within a biological sample by increasing the amount of a target nucleic acid available for analysis. For example, in some forms, the methods amplify a target nucleic acid that includes a sequence of, or complementary to, a target analyte from a sample, such as a biological sample, to enhance the accuracy and/or efficacy of determination of the sequence of the target analyte. In some forms, a method disclosed herein relates to the detection of target nucleic acid sequences (e.g., target RNAs) in situ in one or more cells in a sample wherein background signal is reduced and/or sensitivity of the single cell analysis is increased by the selective amplification of target nucleic acids according to the described methods. In some aspects, the amplification of target nucleic acids according to the described methods results in improved sensitivity (number of detected signals), specificity, signal intensity, and/or improved signal to noise, compared to an equivalent single cell analysis of an equivalent sample wherein target-specific amplification according to the described methods has not been performed.

The methods typically include steps of removing, hybridizing and extending a selectively removable or digestible primer with a strand-displacing DNA polymerase once or more than once to provide a multiplicity of copies of a target DNA molecule. Any of the described methods can further include repeating each of the steps of removing, hybridizing and extending the second primer with a strand-displacing DNA polymerase once or more than once to provide a multiplicity of copies of the target DNA molecule. In some forms, each of the steps of removing, hybridizing and extending the second primer with a strand-displacing DNA polymerase is carried out at a constant temperature.

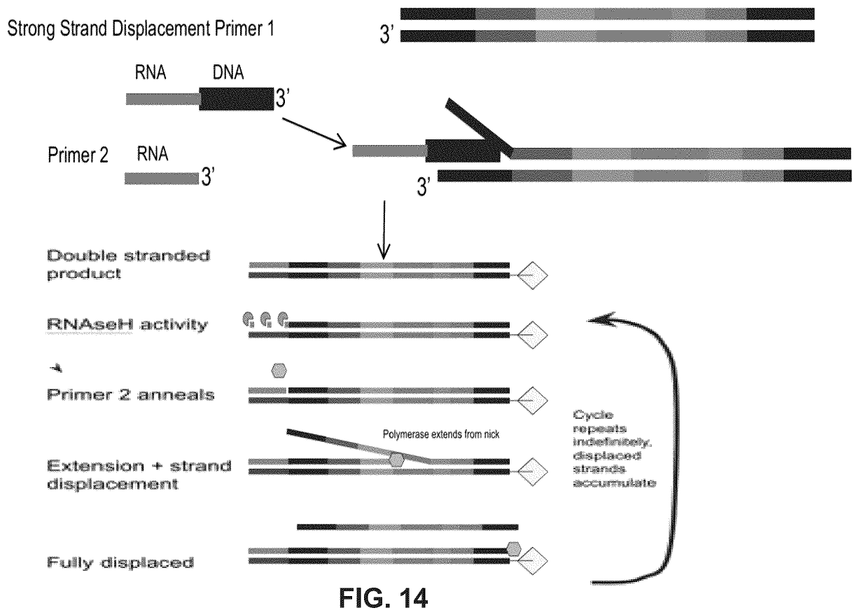

A. Amplification of a Target dsDNA Molecule with a Strongly Complementary, Strand Displacing First Primer (FIG. 14)

Some methods provided herein utilize a first primer that strongly binds its primer binding site sequence in a target dsDNA molecule that is to be amplified. The first primer binds its target binding site with strong enough binding kinetics to invade and displace double stranded DNA during normal dsDNA “breathing” (or “fraying”, i.e., spontaneous local conformational fluctuations within dsDNA which allows strong primer invasion). Invasion by the strong binding primer 1 results displacement of the dsDNA. Additionally, while the primer 1 exhibits strong binding kinetics required for strand displacement, the strong binding primer 1 is still able to be removed during amplification by strand displacing polymerases used in subsequent amplification cycles. In some embodiments, the strong, strand displacing primer 1 may include locked nucleic acids (LNA) and or super G nucleotides to allow design of such primers with appropriately strong binding, wherein the LNA and/or super G content is designed to allow the primer to strongly displace a strand of dsDNA and wherein the bound primer is still itself subsequently displaceable by a strand displacing polymerase.

For example, such methods comprise amplification of a target dsDNA molecule, by (a) hybridizing a first primer (strong binding primer 1 possible comprising LNAs, super G and the like) to a first primer binding site in a target double stranded (ds) DNA molecule, the first primer comprising: (i) a first region, comprising a DNA sequence that is substantially complementary to the first primer binding site of the target DNA molecule, wherein the first region of the first primer displaces dsDNA at the first primer binding site; (ii) a second hybrid region comprising one or more of RNA nucleotides, DNA nucleotides comprising a unique target sequence, or inosine nucleotides.

In some forms, the second region of the first primer exclusively includes RNA nucleotides. In some forms, about 75%, about 80%, about 85%, about 90%, about 95%, about 96%, about 97%, about 98%, about 99% or about 100% of the nucleotides of the second region of the first primer includes RNA nucleotides.

Such methods also include, (b) extending 3′ end of the target DNA molecule using the first primer as a template, to form an extended hybrid double-stranded (ds) DNA molecule; (c) removing the second hybrid region from the extended hybrid dsDNA molecule to form a truncated dsDNA molecule comprising a region of ssDNA; (d) hybridizing a second primer to the region of ssDNA of the truncated dsDNA molecule to form a hybridized second molecule comprising a hybridized second primer, wherein the second primer comprises the second hybrid region comprising one or more of RNA nucleotides, DNA nucleotides comprising a unique target sequence, or inosine nucleotides; and (c) extending the hybridized second primer of the hybridized second molecule using a strand-displacing DNA polymerase to provide an extended hybrid double-stranded (ds) DNA molecule and a displaced single-stranded (ss) DNA molecule.

A target nucleic acid can be any suitable nucleic acid, e.g., preferably DNA (e.g., cDNA, gDNA). The target nucleic acid can be double stranded or single stranded. In some forms, a target nucleic acid includes a barcode, a capture domain, and one or more one or more functional domains (e.g., a sequencing primer binding site, an amplification domain). In some forms, a target nucleic acid includes a barcode, a unique molecular identifier, a capture domain, and one or more one or more functional domains (e.g., a sequencing primer binding site, an amplification domain). In some forms, a target nucleic acid includes a barcode, a unique molecular identifier, a capture domain, a sequence corresponding to a target analyte (e.g., cDNA, ligated RTL probes, an intermediate agent), and one or more functional domains (e.g., a sequencing primer binding site, an amplification domain).

In some forms, two or more of steps (a), (b), (c), (d), and/or (c), are carried out following a single active step, for example, where a single reaction mixture including reagents necessary for two or more of steps (a), (b), (c), (d), and/or (e) is used. In an exemplary form, a single reaction mixture including a first primer (e.g., strong target site binding oligonucleotide primer), a second primer (e.g., oligonucleotide primer), a strand displacing polymerase and an RNase enzyme is used, such that each of steps (a), (b), (c), (d), and/or (c), are carried out following a single active step of contacting the target nucleic acid with the reaction mixture under suitable conditions for a strand displacement reaction (e.g., an isothermal strand displacement) reaction to occur.

Each of these steps is described in greater detail, below, and shown in FIG. 14.

(a) Hybridizing a First Primer to the Target dsDNA

The methods require a step of hybridizing a strong strand displacement first primer (e.g., oligonucleotide primer 1) to the target dsDNA, to bind at the target site for primer 1 and displace a strand of double stranded DNA at the first primer target binding site.

The methods hybridize the first primer which further includes a selectively removable domain to provide (such as an RNA domain) an amplified second strand complementary to the target DNA molecule including the removable domain at the 5′ end, such that removal of the removable domain (such as by RNAse treatment) provides a partially double-stranded DNA molecule having a 5′ “sticky end” that serves as a priming site for a second primer, which in turn includes a selectively removable nucleic acid domain and which is extended by a strand-displacing polymerase.

Optimal binding kinetics for the first primer include the ability to strongly bind the target site of a dsDNA molecule by displacing a strand at the target site during normal dsDNA “breathing”, and permit for the strong binding first primer to still be displaced by strand displacing polymerases. Such optimal binding kinetics may be achieved, as understood by the skilled artisan, by including particular naturally occurring and/or non-naturally occurring (e.g., modified) nucleosides, such as LNAs, super G nucleosides, or the like.

Optimal conditions for hybridizing a first primer to a target nucleic acid can also be determined by one skilled in the art according to the size, composition and quantity of the target nucleic acid and the first primer. Typically, hybridization is carried out under conditions that do not denature or otherwise impact the conformation of the target nucleic acid.

### 1. First Oligonucleotide Primer Design

The strong target binding first oligonucleotide primers for use in the described methods are provided. As used herein, the terms “first primer” and “first oligonucleotide primer” are used interchangeably.

The first primer typically includes at least two functional nucleic acid “domains”, including a first “hybridization” domain and a second “hybrid” (removable) domain. The hybridization domain comprises appropriate natural and modified nucleosides to provide appropriate strong binding kinetics for displacing dsDNA strands at the primer target binding site such primer being itself displaceable by strand displacing polymerases.

The first primer typically includes from about 8 to about 100 nucleotides, inclusive, or any integer (or range of integers) of nucleotides in between the indicated values, for example, between about 16 to about 64 nucleotides, inclusive, or between about 12 to about 24 nucleotides, inclusive. In some forms, the first primer includes about 16 nucleotides. In some embodiments, each of the functional nucleic acid “domains”, includes about 4, 5, 6, 7, 8, 9, 10, 11, 12, 13, 14, 15 or 16, or more nucleotides.

In some forms, the first primer includes more than a single type of nucleotide. For example, in some forms, the first primer includes both DNA and RNA, or DNA and inosine, or DNA and methylated DNA. In other forms, the first primer includes only DNA nucleotides, for example, where a first region of DNA includes a nucleotide sequence that functions to hybridize to the target nucleic acid, and where a second hybrid region includes a DNA nucleotide sequence that functions as an enzyme recognition sequence, such as a restriction endonuclease recognition sequence. Typically, the first primer includes in 5′ to 3′ orientation, the second hybrid region and the first region. When the hybridization domain is located at the 3′ region of the first primer, the second hybrid domain forms a 5′ overhang when the first primer hybridizes to the target nucleic acid (depicted as Strong Strand Displacement Primer 1 in FIG. 14).

In an exemplary form, the size of the first primer is between about 8 and about 100 nucleotides, inclusive, whereby the first region includes between about four and about thirty DNA nucleotides, inclusive, and the second hybrid region includes between about four and about thirty RNA nucleotides, inclusive.

a. First (Hybridization) Domain

The first oligonucleotide primer includes at least one first “hybridization” domain including a nucleotide sequence that is substantially complementary to a nucleotide sequence within a target dsDNA nucleic acid. The first primer hybridization domain provides target binding with strong binding kinetics that invade and displace double stranded DNA during normal dsDNA “breathing” (or “fraying”, i.e., spontaneous local conformational fluctuations within dsDNA which allows strong primer invasion). Additionally, the strong binding primer 1 hybridization domain is still removable during amplification by strand displacing polymerases used in amplification cycles.

The first domain of the first primer is typically formed entirely or partially from DNA and includes a sufficient number of nucleotides to impart specificity for hybridizing to the target nucleic acid. In some forms the first domain of the first primer include natural and modified nucleosides to provide appropriate binding kinetics. In some forms, the sequence that is to be amplified is targeted by the first domain of the first primer. In some embodiments, the first domain includes about 4, 5, 6, 7, 8, 9, 10, 11, 12, 13, 14, 15 or 16, or more than 16 contiguous nucleotides. In an exemplary form, the first domain includes 8 nucleotides. The design of the first domain should include determination of a suitably unique sequence within 3′ end of the target nucleic acid that is of sufficient length to ensure selective hybridization.

Generally, the first hybridization region is or includes any sequence that hybridizes to the 3′ end of the target dsDNA nucleic acid, as required by the described methods. Generally, the target nucleic acid molecule can be any desirable target. In some forms, e.g., where the methods are implemented within one or more single cell analysis methods, the target nucleic acid molecule is the extended capture probe on a substrate such as a bead or particle. For example, in some forms, when the target nucleic acid molecule is a capture probe extended using RNA Templated Ligation (RTL), the first region of the first primer is the same sequence as a first priming site present within a left-hand RTL probe. In an exemplary form, the 3′ end of a target nucleic acid includes a priming site for a first sequencing primer, such as a Read 2S domain. In an exemplary form, where the target nucleic acid includes an extended hybridized capture probe, 3′ end of the target nucleic acid includes a sequence that is complementary to all or a part of a sequencing primer sequence (Read 2S) region of the Left-hand portion of a RTL probe. Therefore, in an exemplary form, the first hybridization domain of the first primer includes a nucleotide sequence of a sequencing primer attachment sequence/binding site. Typically, the first domain is located at the 3′ region of the first primer.

b. Second Hybrid (Removable) Domain

The first oligonucleotide primer includes a second “hybrid” or “removable” domain/region. In some forms, the second hybrid region includes a removable or digestible nucleotide sequence that is or includes a substrate for an enzyme or reaction that digests and/or cleaves all or part of the domain when present in a double-stranded nucleic acid. In some forms, the second hybrid domain of the first primer is formed entirely from RNA and includes a sufficient number of RNA nucleotides that, when removed, provide a region of single-stranded nucleic acid overhang at 3′ end of a ds target nucleic acid, wherein the single-stranded overhang at 3′ end is sufficient for selective hybridization of a second oligonucleotide primer to the single-stranded overhang. In some forms, the second hybrid domain includes about 4, 5, 6, 7, 8, 9, 10, 11, 12, 13, 14, 15 or 16, or more than 16 contiguous nucleotides. In an exemplary form, the second hybrid domain includes 8 nucleotides. The design of the second hybrid domain should include determination of a suitably large sequence to ensure effective removal of the domain by a corresponding enzyme or reagent. Typically, the second hybrid domain is located at 5′ region of the first primer (See primer 2 in FIG. 14).

### 2. Exemplary First Oligonucleotide Primers

Compositions of first oligonucleotide primers and use thereof according to the described methods are provided.

Exemplary first oligonucleotide primers typically include two functional nucleic acid regions, or “domains”, including a first “hybridization” domain and a second “hybrid” (removable) domain. The terms “region” and “domain” are used interchangeably herein to refer to a functional or structural sub-component of a larger nucleic acid. Thus, the term “first domain” of the first primer as used herein is used interchangeably with the term “first region” of the first primer, and the term “second hybrid domain” of the first primer is used interchangeably with the terms “hybrid domain”, “hybrid region”, “second region”, “removable region”, “removable domain”, “second hybrid region”, “second removable region” or “second hybrid region” of the first primer.

Exemplary first oligonucleotide primers include, in 5′ to 3′ orientation, the second hybrid region and the first region (strong primer binding site binder). When the first hybridization domain is located at the 3′ region of the first primer, the second hybrid domain initially forms a 5′ overhang when the first primer hybridizes to the target nucleic acid. The second hybrid domain includes a removable nucleotide sequence that is or includes a substrate for an enzyme or reaction that digests and/or cleaves all or part of the second hybrid domain from within a double-stranded nucleic acid. An exemplary second hybrid domain is formed entirely from RNA and includes a sufficient number of RNA nucleotides that can be removed or digested to provide a region of single-stranded nucleic acids sufficient for selective hybridization of a second oligonucleotide primer to the single-stranded overhang. In some forms, the second hybrid domain includes about 4, 5, 6, 7, 8, 9, 10, 11, 12, 13, 14, 15 or 16, or more than 16 contiguous nucleotides. In an exemplary form, the second hybrid domain includes 8 nucleotides. The design of the second hybrid domain should include determination of a suitably large sequence to ensure effective removal of the domain by a corresponding enzyme or reagent. Typically, the second hybrid domain is located at 5′ region of the first primer.

In an exemplary form, the first oligonucleotide primer includes from about 8 to about 100 nucleotides, inclusive, whereby the first region includes between about four and about thirty DNA nucleotides, inclusive, and the second hybrid region includes between about 4 and about 30 RNA nucleotides, inclusive.

### (b) Extending the First Primer

Following hybridization of the first oligonucleotide primer in step (a), the methods include one or more steps of polymerase-based extension of the primer, using the target nucleic acid as a template (single strand of a dsDNA target), to provide a complementary second nucleic acid strand having a nucleotide sequence complementary to that of the target nucleic acid. In some embodiments, strand displacing polymerases may be used in conjunction with the strand displacing first primers.

Typically, the extension includes use of a polymerase enzyme that is capable of strand displacement (strand displacing polymerase; SDP enzyme). Suitable SDP enzymes for use in the methods include SDP enzymes isolated or derived from Bacillus stearothermophilus such as, Bst2.0 or Bst3.0. The extension initiated by 3′ first hybridization domain/region of the first primer typically yields a second strand that includes the entire first oligonucleotide primer. The extension can also fill in 3′ gap formed by 5′ sticky ended overhang that is created by the hybrid domain of the first primer, to form an extended hybrid double-stranded (ds) DNA molecule. For example, the SDP enzyme can extend the target nucleic acid using the first oligonucleotide primer as a template, thereby generating an extended hybrid double-stranded (ds) DNA molecule. This can occur separately from or concurrently with the extension of the first primer using the target nucleic acid as a template.

Optimal conditions for extension of a first primer using a polymerase, such as an SDP, can be determined by one skilled in the art according to the size, composition and quantity of the target nucleic acid and the first primer. Typically, the extension is carried out under conditions that do not denature or otherwise impact the conformation of the target nucleic acid.

### (c) Removal of the Hybrid Domain

Following extension of the first oligonucleotide primer in step (b), the methods include one or more steps to selectively remove the hybrid domain of the extended hybrid double-stranded (ds) DNA molecule.

Typically, the methods remove the second hybrid region from the second strand amplified in step (b) by contacting the extended hybrid double-stranded (ds) DNA molecule with a suitable reagent to selectively remove the second hybrid region. For example, if the second hybrid region of the first oligonucleotide primer includes RNA, the step of removing the second hybrid region includes contacting the target nucleic acid with an endoribonuclease, e.g., an RNase enzyme, such as an RNase H. In some forms, the RNase H is RNase H1 or RNase H2. An exemplary RNase, (e.g., RNaseH) mediated removal of the hybrid region of an extended hybrid double-stranded (ds) DNA molecule is shown in FIG. 14. Successful removal of the hybrid region provides a partially single stranded, truncated dsDNA molecule that includes a region of ssDNA at the 5′ end of the second (top) strand.

Optimal conditions for removal of a second hybrid region of a first oligonucleotide primer, such as RNA, using a suitable reagent, such as RNaseH, can be determined by one skilled in the art according to the size, composition and quantity of the hybrid region. Typically, the removal is carried out under conditions that do not denature or otherwise impact the conformation of the target nucleic acid.

### (d) Hybridizing a Second Primer to the Target DNA

Following removal of the hybrid region of the extended hybrid double-stranded (ds) DNA molecule in step (c), the methods include one or more steps to hybridize a second oligonucleotide primer to the region of ss nucleic acid of the truncated ds nucleic acid molecule. The second oligonucleotide primer typically is or includes all or part of the second hybrid region of the first oligonucleotide primer e.g., including removable components. In a preferred form, the second primer includes only RNA nucleotides.

Typically, the methods hybridize a second primer, formed entirely of selectively removable nucleotides, to the region of ss nucleic acid of the truncated ds nucleic acid molecule to re-constitute a ds nucleic acid that is amenable to amplification by a strand-displacing polymerase. The second primer hybridizes to the region of ssDNA of the truncated dsDNA molecule. In some forms, this hybridization leaves a nick (e.g., a single-stranded break) at 3′ end of the primer (see, e.g., FIG. 14).

Optimal conditions for hybridizing a second primer to a target nucleic acid can be determined by one skilled in the art according to the size, composition and quantity of the target nucleic acid and the second primer. Typically, hybridization is carried out under conditions that do not denature or otherwise impact the conformation of the target nucleic acid.

### 1. Exemplary Second Oligonucleotide Primers

Compositions of second oligonucleotide primers and uses thereof according to the described methods are also described.

Exemplary second oligonucleotide primers typically include a single removable (“hybrid”) nucleic acid region, or “domain”. The hybrid domain of the second oligonucleotide primer molecule typically includes a removable nucleotide sequence that is or includes a substrate for an enzyme or reaction that digests and/or cleaves all or part of the domain from within a double-stranded nucleic acid.

In some forms, an exemplary second oligonucleotide primer is formed entirely from RNA and includes a sufficient number of RNA nucleotides that, when removed, provide a region of single-stranded (“ss”) nucleic acid sufficient for selective hybridization of a further second oligonucleotide primer to the same single-stranded nucleic acid region.

In some forms, an exemplary second oligonucleotide primer molecule is formed entirely from DNA and inosine nucleotides and includes a sufficient number of inosine nucleotides that, when removed/digested, provide a region of ss nucleic acid sufficient for selective hybridization of a further second oligonucleotide primer to the same single-stranded nucleic acid region.

In some forms, an exemplary second oligonucleotide primer molecule is formed entirely from DNA and includes a sufficient number and sequence of DNA nucleotides that provide a unique recognition site for a restriction endonuclease that, when cleaved, provide a region of ss nucleic acid sufficient for selective hybridization of a further second oligonucleotide primer to the same single-stranded nucleic acid region.

In some forms, an exemplary second oligonucleotide primer is formed entirely from or includes methylated GATC nucleotides that provide a unique recognition site for a methylation-specific enzyme that, upon cleavage of the recognition site, provide a region of ss nucleic acid sufficient for selective hybridization of a further second oligonucleotide primer to the same single-stranded nucleic acid region.

In some forms, the second oligonucleotide primer includes about 4, 5, 6, 7, 8, 9, 10, 11, 12, 13, 14, 15 or 16, or more than 16 contiguous nucleotides. In an exemplary form, the hybrid domain includes 8 nucleotides. The design of the hybrid domain should include determination of a suitably large sequence to ensure effective removal of the domain by a corresponding enzyme or reagent. Typically, the hybrid domain is located at 5′ region of the first primer.

### (e) Extending the Hybridized Second Primer

Following hybridization of a second oligonucleotide primer to the region of ssDNA of the truncated dsDNA molecule in step (d), the methods include one or more steps to extend the hybridized second primer using a strand-displacing DNA polymerase enzyme, to provide an extended hybrid double-stranded (ds) DNA molecule and a displaced single-stranded (ss) DNA molecule that is complementary to the sequence of the target nucleic acid.

The extension reaction in step (e) preferably uses a strand displacing polymerase; SDP enzyme. In some forms, the extension is initiated from 3′ end of the second primer to yield a new (i.e., “third”) nucleic acid strand that displaces the entire second strand that was previously generated in step (b). The extension thereby provides a complete copy of the ds nucleic acid formed in step (b), as well as a single stranded nucleic acid that is complementary to the target nucleic acid (see, e.g., FIG. 14).

Optimal conditions for extension of the second primer using a SDP enzyme can be determined by one skilled in the art according to the size, composition and quantity of the target nucleic acid and the second primer. Typically, the extension is carried out under conditions that do not denature or otherwise impact the conformation of the target nucleic acid.

### Repeating Each of Steps (b), (c), (d), and (e)

In some forms, following one or more steps to extend the hybridized second primer using a strand-displacing DNA polymerase enzyme, to provide an extended hybrid double-stranded (ds) DNA molecule and a displaced single-stranded (ss) DNA molecule that is complementary to the sequence of the target nucleic acid in step (e), the methods include repeating steps (c), (d) and (c) to provide a multiplicity of copies of a displaced single-stranded (ss) DNA molecule that is complementary to the target nucleic acid.

The steps of removing the hybrid domain from the ds nucleic acid by contacting the nucleic acid with a suitable removing reagent to create a single-stranded region, hybridizing a second oligonucleotide primer to the single-stranded region, and then extending the second primer using a suitable SDP enzyme can be repeated once or more, to provide a multiplicity of copies of a displaced single-stranded (ss) DNA molecule that is complementary to the sequence of the target nucleic acid.

In some forms, in the absence of an inhibitory factor, the sequence of steps (a), (d) and (c) are repeated until all available substrates are exhausted. Therefore, in some forms, the amount of a reagent, such as a first primer, a second primer, an endonuclease such as an RNase (e.g., an RNase H), and/or a SDP enzyme that is contacted with a target nucleic acid is configured to provide a desired amount of a displaced single-stranded (ss) DNA molecule that is complementary to the sequence of the target nucleic acid. In other forms, an inhibitor, such as an inhibitor of an RNase H enzyme, or an inhibitor of a SDP enzyme, are applied to stop the further repetition of steps (c), (d) and (c).

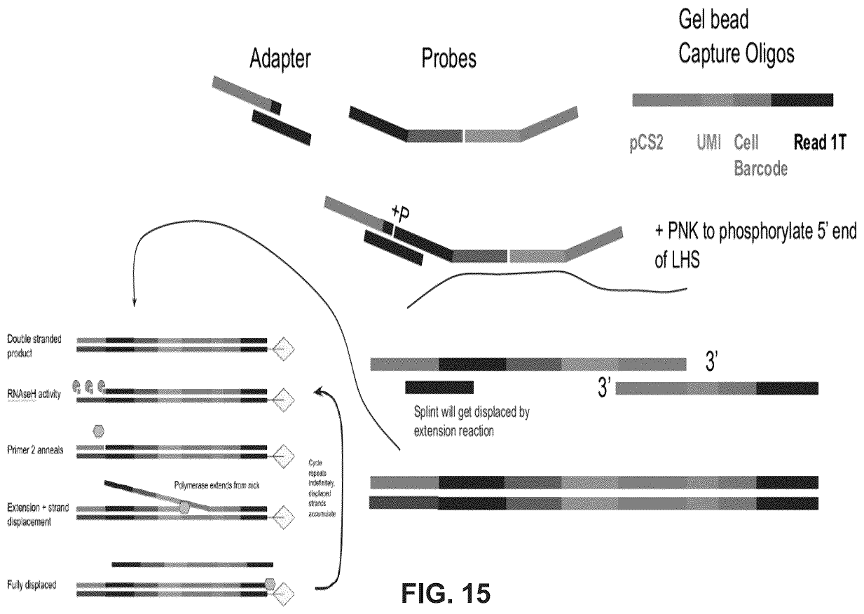

### B. Amplification of a Target DNA Molecule Using a Partially Double Stranded Hybrid DNA/RNA Adapter Oligonucleotide Comprising a Splint Sequence (FIG. 15)

Some methods provided herein utilize a partially double stranded nucleic acid adapter oligonucleotide to form a combined first molecule from a target DNA molecule. The target DNA molecule can be double stranded, partially double stranded or single stranded. In some embodiments, a target DNA molecule may be a single stranded DNA molecule that is modified to a partially double stranded DNA molecule or a double stranded DNA molecule during the executed molecular biological steps utilized to amplify the target DNA. For example an analyte of interest, such as an mRNA transcript of interest, may be initially captured by a capture oligonucleotide or bound by RTL probes. The mRNA transcript may be reverse transcribed into single stranded cDNA, single stranded cDNA can then be used to generate partially double stranded or double stranded DNA molecules with primer extension and/or polymerase reactions to replicate the single stranded DNA and form partially or fully double stranded DNA.

In some embodiments, an RNA Templated Ligation (RTL) approach to target analysis is used as shown in FIG. 15. For example, an mRNA transcript target analyte may be first selected by two primers (right and left) that hybridize to different regions of the mRNA of interest. The primers are extended and ligated to provide ligated RTL probes (see “probes” in FIG. 15). In some embodiments, the RTL probes can be captured by a capture sequence of a capture oligonucleotide. In some embodiments, the capture oligonucleotide may be associated with a substrate such as a bead or particle (see gel bead oligos in FIG. 15). In some embodiments, a partially double stranded DNA/RNA hybrid nucleic acid adapter oligonucleotide comprising a splint sequence is linked to a 5′ end of a strand of partially double stranded target DNA, such as to 5′ end of a ligated RTL probe which in turn may also be hybridized to capture oligonucleotides that may be attached to a substrate such as a bead or a particle. In some embodiments, after hybridization of all of the various portions of nucleic acids (i.e., adapters, probes, and capture oligonucleotides, etc.) extension reactions may be performed to fill in any single stranded portions and provide a double stranded product for amplification by methods described herein (FIG. 15).

In some embodiments, a partially double stranded DNA/RNA hybrid adapter comprising a splint sequence is linked to the 5′ end of a target DNA, such as a ligated RTL probe, as shown in FIG. 15. In such embodiments, the splint sequence hybridizes to the ligated RTL probe. In some embodiments, the splint is then displaced during extension reactions wherein said extension reaction also transforms the combined adapter, RTL probe and capture oligo molecule into a double stranded DNA molecule as shown in FIG. 15 that can be amplified by the strand displacement methodologies provided herein. In such embodiments, the double dsDNA molecule also includes a hybrid region that can be removed to form a truncated DNA molecule comprising a region of ssDNA. For example, the hybrid region may comprise one strand of RNA nucleotides that can be removed with an RNase to provide the truncated DNA molecule comprising a region of ssDNA. A second primer can be hybridized to the ssDNA region in order to amplify the DNA. In some embodiments, the second primer comprises comprising RNA nucleotides, DNA nucleotides comprising a unique target sequence, or inosine nucleotides and can be removed as needed to provide additional regions of ssDNA that may be used for further priming, extension and amplification. In some embodiments, extending the hybridized second primer is performed with a strand-displacing DNA polymerase to provide an extended hybrid dsDNA molecule and a displaced DNA molecule (FIG. 15).

For example, some embodiments include methods that comprise amplification of a target DNA molecule, by forming a combined first molecule from a target partially double stranded (ds) DNA molecule comprising a first DNA strand and a second DNA strand, the forming comprising:
(i) linking to a 5′ end of the second DNA strand of the target dsDNA molecule a partially double stranded nucleic acid adapter, the nucleic acid adapter comprising:
   (I) a first adapter strand, comprising:
      (i) a first region, comprising DNA nucleotides; and
      (ii) a second hybrid 5′ overhang region, comprising RNA nucleotides, or DNA nucleotides comprising a unique target sequence, or inosine nucleotides; and
   (II) a second adapter strand comprising a 3′ overhang DNA splint sequence, wherein the splint sequence is complementary to all or part of the first adapter strand and 5′ end of the second DNA strand of the target partially dsDNA molecule; wherein the first adapter strand is hybridized to the second adapter strand; and
      (i) extending 3′end of the second DNA strand of the target dsDNA molecule using the first adapter strand as a template, wherein extending the 3′end of the second DNA strand of the target dsDNA molecule displaces the DNA splint sequence.

In some forms, the second hybrid 5′ overhang region of the partially double stranded nucleic acid adapter exclusively includes RNA nucleotides. In some forms, about 75%, about 80%, about 85%, about 90%, about 95%, about 96%, about 97%, about 98%, about 99% or about 100% of the nucleotides of the second region of the first primer includes RNA nucleotides.

In some embodiments, methods also include, (i) extending 3′end of the second DNA strand of the target dsDNA molecule using the first adapter strand as a template, wherein extending 3′end of the second DNA strand of the target dsDNA molecule displaces the DNA splint sequence.

In some embodiments, the methods also include:
(b) removing the second hybrid region from the combined first molecule to form a truncated DNA molecule comprising a region of ssDNA;
(c) hybridizing a second primer to the region of ssDNA of the truncated DNA molecule to form a hybridized second molecule comprising a hybridized second primer, wherein the second primer comprises the second hybrid region comprising RNA nucleotides, DNA nucleotides comprising a unique target sequence, or inosine nucleotides; and
(d) extending the hybridized second primer with a strand-displacing DNA polymerase to provide an extended hybrid dsDNA molecule and a displaced DNA molecule.

A target nucleic acid can be any suitable nucleic acid, e.g., preferably DNA (e.g., cDNA, gDNA). The target nucleic acid can be double stranded, partially double stranded or single stranded. In some forms, a target nucleic acid includes a barcode, a capture domain, and one or more one or more functional domains (e.g., a sequencing primer binding site, an amplification domain). In some forms, a target nucleic acid includes a barcode, a unique molecular identifier, a capture domain, and one or more one or more functional domains (e.g., a sequencing primer binding site, an amplification domain). In some forms, a target nucleic acid includes a barcode, a unique molecular identifier, a capture domain, a sequence corresponding to a target analyte (e.g., cDNA, ligated RTL probes, an intermediate agent), and one or more functional domains (e.g., a sequencing primer binding site, an amplification domain).

In some forms, two or more of steps (a), (b), (c), and/or (d) are carried out following a single active step, for example, where a single reaction mixture including reagents necessary for two or more of steps (a), (b), (c), and/or (d) is used. In an exemplary form, a single reaction mixture including a partially double stranded nucleic acid adapter oligonucleotide (e.g., RNA/DNA hybrid oligonucleotide with), a second primer (e.g., partially double stranded oligonucleotide primer comprising a splint sequence), a strand displacing polymerase and an RNAse enzyme is used, such that each of steps (a), (b), (c), and/or (d), are carried out following a single active step of contacting the target nucleic acid with the reaction mixture under suitable conditions for a strand displacement reaction (e.g., an isothermal strand displacement) reaction to occur.

Each of these steps is described in greater detail, below, and shown in FIG. 15.

(a) Forming a Combined First Molecule from a Target Partially Double Stranded (Ds) DNA Molecule Comprising a First DNA Strand and a Second DNA Strand

A partially double stranded DNA/RNA hybrid adapter comprising a splint sequence is provided and linked to the 5′ end of a target DNA, such as a ligated RTL probe, as shown in FIG. 15. In such embodiments, the splint sequence hybridizes to the ligated RTL probe. In some embodiments, the splint is then displaced during extension reactions wherein said extension reaction also transforms the combined adapter, RTL probe and capture oligo molecule into a double stranded DNA molecule as shown in FIG. 15 that can be amplified by the strand displacement methodologies provided herein. The methods hybridize the adapter which further includes a selectively removable domain to provide (such as an RNA domain) an amplified second strand complementary to the target DNA molecule including the removable domain at the 5′ end, such that removal of the removable domain (such as by RNAse treatment) provides a partially double-stranded DNA molecule having a 5′ “sticky end” that serves as a priming site for a second primer, which in turn includes a selectively removable nucleic acid domain and which is extended by a strand-displacing polymerase.

In some embodiments, the adapter includes two strands hybridized to each other, wherein a (I) first adapter strand comprises
(i) a first region, comprising DNA nucleotides
(ii) a second hybrid 5′ overhang region, comprising RNA nucleotides, or DNA nucleotides comprising a unique target sequence, or inosine nucleotides; and
(II) a second adapter strand comprising a 3′ overhang DNA splint sequence, wherein the splint sequence is complementary to all or part of the first adapter strand and 5′ end of the second DNA strand of the target partially dsDNA. Thereby the splint sequence provides compatibility for hybridization of the second adapter strand to the first adapter strand and also provides compatibility for hybridization to the target DNA in order to form the partially double stranded molecule comprising the adapter, the target (such as ligated RTL probes in FIG. 15) and the capture oligonucleotide, which in some embodiments is attached to a substrate such as a bead (including gel beads) or particle.

Optimal binding kinetics for the adapter include the ability of the splint region to bind the target DNA. Such optimal binding kinetics may be achieved, as understood by the skilled artisan.

Optimal conditions for hybridizing the partially double stranded adapter with splint sequence to a target nucleic acid can also be determined by one skilled in the art according to the size, composition and quantity of the target nucleic acid and the adapter. Typically, hybridization is carried out under conditions that do not denature or otherwise impact the conformation of the target nucleic acid.

### 1. Adapter Design

The partially double stranded adapter with splint sequence typically includes at least two functional nucleic acid “domains”, including a splint hybridization domain and a second “hybrid” (removable) domain. The splint hybridization domain comprises appropriate natural and modified nucleosides to provide appropriate binding kinetics for the two strands of the adapter to interact with each other.

The adapter typically includes from about 8 to about 100 nucleotides, inclusive, or any integer (or range of integers) of nucleotides in between the indicated values, for example, between about 16 to about 64 nucleotides, inclusive, or between about 12 to about 24 nucleotides, inclusive. In some forms, the first primer includes about 16 nucleotides. In some embodiments, each of the functional nucleic acid “domains”, includes about 4, 5, 6, 7, 8, 9, 10, 11, 12, 13, 14, 15 or 16, or more nucleotides.

In some forms, the partially double stranded adapter with splint sequence includes more than a single type of nucleotide. For example, in some forms, the partially double stranded adapter with splint sequence includes both DNA and RNA, or DNA and inosine, or DNA and methylated DNA. In other forms, the partially double stranded adapter with splint sequence includes only DNA nucleotides, for example, where a first region of DNA includes a nucleotide sequence that functions to hybridize to the target nucleic acid, and where a second hybrid region includes a DNA nucleotide sequence that functions as an enzyme recognition sequence, such as a restriction endonuclease recognition sequence.

In an exemplary form, the size of the partially double stranded adapter with splint sequence is between about 8 and about 100 nucleotides, inclusive, whereby the first stand includes between about four and about thirty DNA nucleotides, inclusive, and the second region includes about four and about thirty RNA nucleotides, inclusive. In embodiments, the second strand comprises a splint sequence for hybridizing to the target DNA and the first stand of the adapter.

a. Adapter First Strand

In some embodiments, the partially double stranded adapter with splint sequence has a first strand that includes two domains wherein the first domain includes a nucleotide sequence that is substantially complementary to a splint sequence present in the second strand of the adapter. The splint hybridization domain provides sequences for hybridization with the second strand comprising a splint sequence. In some embodiments, the first adapter strand comprises (i) a first region, comprising DNA nucleotides (ii) a second hybrid 5′ overhang region, comprising RNA nucleotides, or DNA nucleotides comprising a unique target sequence, or inosine nucleotides.

The first region of the partially double stranded adapter first strand is typically formed entirely or partially from DNA and includes a sufficient number of nucleotides to impart specificity for hybridizing to the second adapter strand. In some forms the first region of the partially double stranded adapter first strand includes natural and modified nucleosides to provide appropriate binding kinetics. In some embodiments, the first region includes about 4, 5, 6, 7, 8, 9, 10, 11, 12, 13, 14, 15 or 16, or more than 16 contiguous nucleotides. In an exemplary form, the first domain includes 8 nucleotides. The design of the first region should include determination of a suitably unique sequence within 3′ end of the target nucleic acid that is of sufficient length to ensure selective hybridization with the second strand of the adapter. Generally, the first hybridization region is or includes any sequence that hybridizes to the second adapter strand and/or splint sequence, as required by the described methods.

The first strand of the partially double stranded adapter with splint sequence also includes a second “hybrid” or “removable” region. In some forms, the second hybrid region includes a removable or digestible nucleotide sequence that is or includes a substrate for an enzyme or reaction that digests and/or cleaves all or part of the domain when present in a double-stranded nucleic acid. In some forms, the second hybrid region of the partially double stranded adapter with splint sequence is formed entirely from RNA and includes a sufficient number of RNA nucleotides that, when removed, provide a region of single-stranded nucleic acid overhang at 3′ end of a ds target nucleic acid, wherein the single-stranded overhang at 3′ end is sufficient for selective hybridization of a second oligonucleotide primer to the single-stranded overhang. In some forms, the second hybrid region includes about 4, 5, 6, 7, 8, 9, 10, 11, 12, 13, 14, 15 or 16, or more than 16 contiguous nucleotides. In an exemplary form, the second hybrid region includes 8 nucleotides. The design of the second hybrid region should include determination of a suitably large sequence to ensure effective removal of the domain by a corresponding enzyme or reagent. Typically, the second hybrid region is located at 5′ region of the first strand of the adapter (See Adapter in FIG. 15).

b. Adapter Second Strand

In some embodiments, the partially double stranded adapter with splint sequence has a second strand that includes a splint sequence comprising sequences for hybridization to the first adapter strand and the target, such as a ligated RTL probe target. In some embodiments, the second adapter strand comprises a 3′ overhang DNA splint sequence, wherein the splint sequence is complementary to all or part of the first adapter strand and 5′ end of the target, such as a target nucleic acid, including second DNA strands of partially target dsDNA.

In some embodiments, the second strand of the partially double stranded adapter comprises a splint sequence that is typically formed entirely or partially from DNA and includes a sufficient number of nucleotides to impart specificity for hybridizing to the first adapter strand and the target nucleic acid (see FIG. 15). In some forms the second strand of the partially double stranded adapter includes splint sequences comprising natural and modified nucleosides to provide appropriate binding kinetics to bind both the first adapter stand and the target nucleic acid in a manner that links or “splints” the first strand and the target through the second strand splint sequence. In some embodiments, the second strand splint sequence includes about 4, 5, 6, 7, 8, 9, 10, 11, 12, 13, 14, 15 or 16, or more than 16 contiguous nucleotides. In an exemplary form, the second adapter strand includes 8 nucleotides. The design of the second strand splint sequence should include determination of a suitably unique sequence within 3′ end of the target nucleic acid that is of sufficient length to ensure selective hybridization with the second strand of the adapter. Generally, the first hybridization region is or includes any sequence that hybridizes to the first adapter strand and/or target nucleic acid sequence, as required by the described methods.

(a)(i) Extending 3′end of the Second DNA Strand of the Target dsDNA Molecule Using the First Adapter Strand as a Template, Wherein Extending the 3′End of the Second DNA Strand of the Target dsDNA Molecule Displaces the DNA Splint Sequence.

Following forming a combined first molecule from a target nucleic acid, such as a target partially double stranded (ds) DNA molecule, and the adapter in step (a), the methods include one or more steps of polymerase-based extension of the target dsDNA molecule using the first adapter strand as a template, wherein extending 3′end of the second DNA strand of the target dsDNA molecule displaces the DNA splint sequence. In some embodiments, strand displacing polymerases may be used.

Typically, the extension includes use of a polymerase enzyme, including standard polymerases and/or a polymerase that is capable of strand displacement (strand displacing polymerase; SDP enzyme). Suitable SDP enzymes for use in the methods include SDP enzymes isolated or derived from Bacillus stearothermophilus such as, Bst2.0 or Bst3.0. The extension typically yields a fully double stranded nucleic acid target, such as dsDNA, that can be further amplified by methods provided herein (see FIG. 15.)

Optimal conditions for extension of a first primer using a polymerase, such as an SDP, can be determined by one skilled in the art according to the size, composition and quantity of the target nucleic acid and the first primer. Typically, the extension is carried out under conditions that do not denature or otherwise impact the conformation of the target nucleic acid.

### (b) Removal of the Hybrid Region

Following extension of the adapter in step (a)(i), the methods include one or more steps to selectively remove the hybrid domain of the extended hybrid double-stranded (ds) DNA molecule.

Typically, the methods remove the hybrid region from the second strand amplified in step (a) by contacting the extended hybrid double-stranded (ds) DNA molecule with a suitable reagent to selectively remove the second hybrid region. For example, if the hybrid region of the first oligonucleotide primer includes RNA, the step of removing the hybrid region includes contacting the target nucleic acid with an endoribonuclease, e.g., an RNase enzyme, such as an RNase H. In some forms, the RNase H is RNase H1 or RNase H2. An exemplary RNase, (e.g., RNaseH) mediated removal of the hybrid region of an extended hybrid double-stranded (ds) DNA molecule is shown in FIG. 15. Successful removal of the hybrid region provides a partially single stranded, truncated dsDNA molecule that includes a region of ssDNA at the 5′ end of the second (top) strand.

Optimal conditions for removal of a hybrid region of a first oligonucleotide primer, such as RNA, using a suitable reagent, such as RNaseH, can be determined by one skilled in the art according to the size, composition and quantity of the hybrid region. Typically, the removal is carried out under conditions that do not denature or otherwise impact the conformation of the target nucleic acid.

### (c) Hybridizing a Second Primer to the Ss Region of Target DNA

Following removal of the hybrid region of the extended hybrid double-stranded (ds) DNA molecule in step (b), the methods include one or more steps to hybridize a second oligonucleotide primer to the region of ss nucleic acid of the truncated ds nucleic acid molecule. The second oligonucleotide primer typically is or includes all or part of the second hybrid region of the first oligonucleotide primer e.g., including removable components. In a preferred form, the second primer includes only RNA nucleotides.

Typically, the methods hybridize a second primer, formed entirely of selectively removable nucleotides, to the region of ss nucleic acid of the truncated ds nucleic acid molecule to re-constitute a ds nucleic acid that is amenable to amplification by a strand-displacing polymerase. The second primer hybridizes to the region of ssDNA of the truncated dsDNA molecule. In some forms, this hybridization leaves a nick (i.e., a single-stranded break) at 3′ end of the primer (see, e.g., FIG. 15).

Optimal conditions for hybridizing a second primer to a target nucleic acid can be determined by one skilled in the art according to the size, composition and quantity of the target nucleic acid and the second primer. Typically, hybridization is carried out under conditions that do not denature or otherwise impact the conformation of the target nucleic acid.

### 1. Exemplary Second Oligonucleotide Primers

Compositions of second oligonucleotide primers and uses thereof according to the described methods are also described.

Exemplary second oligonucleotide primers typically include a single removable (“hybrid”) nucleic acid region, or “domain”. The hybrid domain of the second oligonucleotide primer molecule typically includes a removable nucleotide sequence that is or includes a substrate for an enzyme or reaction that digests and/or cleaves all or part of the domain from within a double-stranded nucleic acid.

In some forms, an exemplary second oligonucleotide primer is formed entirely from RNA and includes a sufficient number of RNA nucleotides that, when removed, provide a region of single-stranded (“ss”) nucleic acid sufficient for selective hybridization of a further second oligonucleotide primer to the same single-stranded nucleic acid region.

In some forms, an exemplary second oligonucleotide primer molecule is formed entirely from DNA and inosine nucleotides and includes a sufficient number of inosine nucleotides that, when removed/digested, provide a region of ss nucleic acid sufficient for selective hybridization of a further second oligonucleotide primer to the same single-stranded nucleic acid region.

In some forms, an exemplary second oligonucleotide primer molecule is formed entirely from DNA and includes a sufficient number and sequence of DNA nucleotides that provide a unique recognition site for a restriction endonuclease that, when cleaved, provide a region of ss nucleic acid sufficient for selective hybridization of a further second oligonucleotide primer to the same single-stranded nucleic acid region.

In some forms, an exemplary second oligonucleotide primer is formed entirely from or includes methylated GATC nucleotides that provide a unique recognition site for a methylation-specific enzyme that, upon cleavage of the recognition site, provide a region of ss nucleic acid sufficient for selective hybridization of a further second oligonucleotide primer to the same single-stranded nucleic acid region.

In some forms, the second oligonucleotide primer includes about 4, 5, 6, 7, 8, 9, 10, 11, 12, 13, 14, 15 or 16, or more than 16 contiguous nucleotides. In an exemplary form, the hybrid domain includes 8 nucleotides. The design of the hybrid domain should include determination of a suitably large sequence to ensure effective removal of the domain by a corresponding enzyme or reagent. Typically, the hybrid domain is located at 5′ region of the first primer.

### (d) Extending the Hybridized Second Primer

Following hybridization of a second oligonucleotide primer to the region of ssDNA of the truncated dsDNA molecule in step (c), the methods include one or more steps to extend the hybridized second primer using a strand-displacing DNA polymerase enzyme, to provide an extended hybrid double-stranded (ds) DNA molecule and a displaced DNA molecule that is complementary to the sequence of the target nucleic acid.

The extension reaction in step (d) preferably uses a strand displacing polymerase; SDP enzyme. In some forms, the extension is initiated from 3′ end of the second primer to yield a new (i.e., “third”) nucleic acid strand that displaces the entire second strand that was previously generated in step (b). The extension thereby provides a complete copy of the ds nucleic acid formed in step (b), as well as a single stranded nucleic acid that is complementary to the target nucleic acid (see, e.g., FIG. 15).

Optimal conditions for extension of the second primer using a SDP enzyme can be determined by one skilled in the art according to the size, composition and quantity of the target nucleic acid and the second primer. Typically, the extension is carried out under conditions that do not denature or otherwise impact the conformation of the target nucleic acid.

Repeating Each of Steps (a)(i), (b), (c), and (d)

In some forms, following one or more steps to extend the hybridized second primer using a strand-displacing DNA polymerase enzyme, to provide an extended hybrid double-stranded (ds) DNA molecule and a displaced single-stranded (ss) DNA molecule that is complementary to the sequence of the target nucleic acid in step (d), the methods include repeating steps (b), (c) and (d) to provide a multiplicity of copies of a displaced single-stranded (ss) DNA molecule that is complementary to the target nucleic acid.

The steps of removing the hybrid domain from the ds nucleic acid by contacting the nucleic acid with a suitable removing reagent to create a single-stranded region, hybridizing a second oligonucleotide primer to the single-stranded region, and then extending the second primer using a suitable SDP enzyme can be repeated once or more, to provide a multiplicity of copies of a displaced single-stranded (ss) DNA molecule that is complementary to the sequence of the target nucleic acid.

In some forms, in the absence of an inhibitory factor, the sequence of steps (a), (c) and (d) are repeated until all available substrates are exhausted. Therefore, in some forms, the amount of a reagent, such as an adapter, a second primer, an endonuclease such as an RNase (e.g., an RNase H), and/or a SDP enzyme that is contacted with a target nucleic acid is configured to provide a desired amount of a displaced single-stranded (ss) DNA molecule that is complementary to the sequence of the target nucleic acid. In other forms, an inhibitor, such as an inhibitor of an RNase H enzyme, or an inhibitor of a SDP enzyme, are applied to stop the further repetition of steps (b), (c) and (d).

### V. Kits, Systems and Compositions for Strand-Displacement Amplification of Target Nucleic Acids

Provided herein are kits and systems that include one or more compositions and/or reagents for performing the described methods of amplifying a target nucleic acid, for example, within or following a single cell analysis workflow. In embodiments, components, hardware, software and the like for performing the disclosed methods are also provided.

In some aspects, provided herein are compositions comprising any of the reagents for performing extension by a strand displacing polymerase (SDP) using a ssDNA region as template to amplify a target nucleic acid, and oligonucleotide primers and/or adapter molecules (e.g., primers, double stranded adapter molecules or partially double stranded adapter molecules), and/or intermediate molecules described herein.

Also provided herein are kits, systems or compositions for amplifying a target nucleic acid in a biological sample according to any of the methods described herein. In some embodiments, provided herein is a kit, system or composition may include any of the nucleic acid oligonucleotides described herein (e.g., first and/or second primers for extension using ssDNA as template, partially double stranded DNA/RNA hybrid primers with splint sequences, etc.). In some embodiments, the kit, system or composition further includes any of the reagents, oligonucleotides, arrays, enzymes or nucleic acids disclosed herein. In some embodiments, the kit includes a removable region-specific enzyme, e.g., an endonuclease such as RNaseH.

The various components of the kit, system or composition may be present in separate containers or certain compatible components may be pre-combined into a single container. In some embodiments, the kits further contain instructions for using the components of the kit to practice the provided methods.

A primer and/or adapter set for use with the described methods is provided. Typically, the primer set includes: (a) a first primer or adapter, including (i) a first region including DNA bases complementary to a first primer binding site of a target nucleic acid or a splint sequence wherein the splint sequence binds the target; and (ii) a second hybrid region, wherein the second region includes one or more of RNA bases, DNA bases including a unique target sequence, inosine bases, or a methylated GATC sequence; and (b) a second primer, including the second hybrid region of the first primer.

In some forms, the second primer is formed entirely of the second hybrid region of the first primer. An exemplary first primer is between about 8 and about 100 bases, inclusive. In some forms, the first primer includes a first region including between about four and about thirty bases, inclusive. In other forms, the first primer includes a second region including between about four and about thirty bases, inclusive. In some forms, the first region of the first primer includes one or more locked nucleic acids (LNA). In some forms, the second region of the first primer includes RNA nucleotides. In other forms, the second region of the first primer includes inosine nucleotides. In certain forms, the second region of the first primer includes a unique target sequence, for example, a target sequence including a nickase recognition sequence. In other forms, the second region of the first primer includes a methylated GATC sequence.

Specific kits, systems and/or compositions including specific reagents for performing the described methods are also provided. In some forms, a kit includes one or more of: (a) a first primer or adapter, including (i) a first region including DNA bases complementary to a first primer binding site of a target nucleic acid or to a splint sequence wherein the splint sequence is further complementary to a target sequence; and (ii) a second hybrid region, whereby the second region includes one or more of RNA bases, DNA bases including a unique target sequence, inosine bases, or a methylated GATC sequence; and (b) instructions for performing the described methods for amplifying a target nucleic acid.

In some forms, the second region of the first primer includes RNA bases.

In some forms, the kits, systems or compositions further include a second primer including the second region of the first primer. In some forms, the kit further includes an RNA-specific nuclease enzyme, such as an RNase enzyme, such as RNaseH.

In some forms, the second region of the first primer includes inosine nucleotides. In some forms, the kits, systems or compositions include a second primer including the inosine nucleotides of the second region of the first primer. In some forms, the kits, systems or compositions further include an endonuclease enzyme that degrades the inosine nucleotides. An exemplary endonuclease enzyme includes Endonuclease V.

In some forms, the second region of the first primer or a region of an adapter includes one or more unique target sequences. In some forms, the kit further includes a second primer including the unique target sequences region of the first primer. In some forms, the kit further includes a nickase enzyme that selectively cleaves or removes the unique target sequences.

In some forms, the second region of the first primer or a region of an adapter includes one or more methylated GATC sequences. In some forms, the kits, systems or compositions further include a second primer including the one or more methylated GATC sequences of the first primer. In some forms, the kit further includes a methylation-specific nuclease enzyme. An exemplary methylation-specific nuclease enzyme includes DpnI.

In other forms, the kits, systems or compositions include a single stranded adapter, a partially double stranded adapter, or a double stranded adapter.

An exemplary adapter includes a first strand with nucleotides complementary to the second region of the first primer and/or a hybrid region. In some forms, the adapter comprises a second strand comprising a splint sequence that hybridizes to a target nucleic acid and to the first strand of the adapter. In some forms, the first adapter includes a 5′ phosphate on the second strand.

In some forms, the kits, systems or compositions further include a ligase enzyme. An exemplary ligase enzyme includes a T4 ligase.

In some forms, the kits, systems or compositions further include a strand displacing DNA polymerase, such as a Bst2.0, Bst3.0, Bsu or Phil29 strand displacing DNA polymerase.

In some forms, the kits, systems or compositions further include one or more of (a) a solvent suitable to solubilize unbound first primer(s) and/or unbound second primer(s); (b) one or more DNA-specific Polymerase enzymes; (c) one or more nuclease enzymes; and/or (d) buffers and/or reagents suitable for enzyme reactions according to the described methods for amplifying a nucleic acid.

The disclosed compositions, kits, systems, and methods can be further understood through the following numbered paragraphs.

1. A method for amplification of a target DNA molecule, comprising:
(a) hybridizing a first primer to a first primer binding site in a target double stranded (ds) DNA molecule, the first primer comprising:
   (i) a first region, comprising a DNA sequence that is substantially complementary to the first primer binding site of the target DNA molecule, wherein the first region of the first primer displaces dsDNA at the first primer binding site;
   (ii) a second hybrid region comprising one or more of RNA nucleotides, DNA nucleotides comprising a unique target sequence, or inosine nucleotides;
(b) extending 3′ end of the target DNA molecule using the first primer as a template, to form an extended hybrid double-stranded (ds) DNA molecule;
(c) removing the second hybrid region from the extended hybrid dsDNA molecule to form a truncated dsDNA molecule comprising a region of ssDNA;
(d) hybridizing a second primer to the region of ssDNA of the truncated dsDNA molecule to form a hybridized second molecule comprising a hybridized second primer,
   wherein the second primer comprises the second hybrid region comprising one or more of RNA nucleotides, DNA nucleotides comprising a unique target sequence, or inosine nucleotides; and
(e) extending the hybridized second primer of the hybridized second molecule using a strand-displacing DNA polymerase to provide an extended hybrid double-stranded (ds) DNA molecule and a displaced single-stranded (ss) DNA molecule.
   2 The method of paragraph 1, wherein the first region comprises one or more modified nucleic acids.
   3. The method of paragraph 1 or 2, wherein the first region comprises one or more locked nucleic acids (LNAs).
   4. The method of any one of the preceding paragraphs, wherein the first region comprises one or more super G (8-aza-7-deazaguanosine) nucleic acids.
   5. The method of any one of the preceding paragraphs, wherein the first region is located at 3′ end of the first primer.
   6. The method of any one of the preceding paragraphs, wherein the second hybrid region of the first primer forms a single stranded 5′ overhang when the first primer hybridizes to the target DNA molecule.
   7. The method of any one of the preceding paragraphs, wherein the first primer comprises in 5′ to 3′ orientation: the second hybrid region and the first region.
   8. A method for amplification of a target DNA molecule, comprising:
(a) forming a combined first molecule from a target partially double stranded (ds) DNA molecule comprising a first DNA strand and a second DNA strand, the forming comprising:
   (i) linking to a 5′ end of the second DNA strand of the target dsDNA molecule a partially double stranded nucleic acid adapter, the nucleic acid adapter comprising:
      (I) a first adapter strand, comprising:
         (i) a first region, comprising DNA nucleotides; and
         (ii) a second hybrid 5′ overhang region, comprising RNA nucleotides, or DNA nucleotides comprising a unique target sequence, or inosine nucleotides;
      and
      (II) a second adapter strand comprising a 3′ overhang DNA splint sequence, wherein the splint sequence is complementary to all or part of the first adapter strand and 5′ end of the second DNA strand of the target partially dsDNA molecule;
      wherein the first adapter strand is hybridized to the second adapter strand; and
   (i) extending 3′end of the second DNA strand of the target dsDNA molecule using the first adapter strand as a template, wherein extending the 3′end of the second DNA strand of the target dsDNA molecule displaces the DNA splint sequence;
(b) removing the second hybrid region from the combined first molecule to form a truncated DNA molecule comprising a region of ssDNA;
(c) hybridizing a second primer to the region of ssDNA of the truncated DNA molecule to form a hybridized second molecule comprising a hybridized second primer, wherein the second primer comprises the second hybrid region comprising RNA nucleotides, DNA nucleotides comprising a unique target sequence, or inosine nucleotides;
(d) extending the hybridized second primer with a strand-displacing DNA polymerase to provide an extended hybrid dsDNA molecule and a displaced DNA molecule.
   9. The method of any one of the preceding paragraphs, wherein the target DNA molecule is directly or indirectly conjugated to a particle.
   10. The method of paragraph 9, where the particle is a bead.
   11. The method of paragraph 9 or 10, wherein the particle is a gel bead.
   12. The method of any one of paragraphs 9-10, wherein the particle comprises a barcode sequence.
   13. The method of any one of the preceding paragraphs, wherein the displaced DNA molecule comprises a sequence of, or complementary to that of the target DNA molecule.
   14. The method of any one of the preceding paragraphs, wherein the strand-displacing DNA polymerase is selected from the group consisting of Bst2.0, Bst3.0, Bsu and Phil29.
   15. The method of any one of the preceding paragraphs, wherein removing the second hybrid region comprises contacting the second hybrid region with an enzyme that selectively degrades the second hybrid region.
   16. The method of paragraph 15, wherein the second hybrid region comprises RNA nucleotides, and wherein removing the second hybrid region comprises contacting the second hybrid region with an RNase enzyme.
   17. The method of paragraph 16, wherein the RNase enzyme comprises RNase H.
   18. The method of paragraph 15, wherein the second hybrid region comprises one or more inosine nucleotides, and wherein removing the second hybrid region comprises contacting the second hybrid region with an endonuclease enzyme that cleaves the one or more inosine nucleotides.
   19. The method of paragraph 18, wherein the endonuclease comprises Endonuclease V.
   20. The method of paragraph 15, wherein the second hybrid region comprises one or more unique target sequences, wherein the one or more unique target sequences are not present within the target DNA molecule, and wherein removing the second hybrid region comprises contacting the second hybrid region with a nickase enzyme that selectively cleaves or removes the one or more unique target sequences.
   21. The method of paragraph 15, wherein the second hybrid region comprises one or more methylated GATC sequences, and wherein removing the second hybrid region comprises contacting the second region with a methylation-specific nuclease.
   22. The method of paragraph 21, wherein the methylation-specific nuclease comprises DpnI.
   23. The method of any one of the preceding paragraphs, wherein forming in step (a) comprises contacting the target DNA molecule with a reaction mixture comprising the first primer, or partially double stranded adapter, and the strand displacing DNA polymerase.
   24. The method of paragraph 23, wherein the reaction mixture further comprises one or more of:
(i) an enzyme specific for the second hybrid region;
(ii) and the second primer.
   25. The method of any one of the preceding paragraphs, further comprising repeating each of the steps of removing, hybridizing and extending the second primer with a strand-displacing DNA polymerase once or more than once to provide a multiplicity of copies of the target DNA molecule.
   26. The method of any one of the preceding paragraphs, wherein each of the steps of removing, hybridizing and extending the second primer with a strand-displacing DNA polymerase is carried out at a constant temperature.
   27. The method of any one of the preceding paragraphs, wherein the target DNA molecule comprises a barcode and/or a DNA sequence of at least a portion of a target nucleic acid analyte from a sample, or a complement thereof.
   28. The method of paragraph 27, wherein the target nucleic acid analyte comprises a nucleic acid, optionally wherein the analyte is selected from genomic DNA, RNA, synthetic DNA, or synthetic RNA.
   29. The method of paragraph 28, wherein the target nucleic acid analyte is derived from a biological sample, optionally wherein the biological sample is derived from a human subject.
   30. The method of paragraph 29, wherein the target nucleic acid analyte comprises an RNA selected from the group consisting of small interfering RNA (siRNA), microRNA (miRNA), P-element-induced wimpy testis (PIWI)-interacting RNA (piRNA), small nucleolar RNA (snoRNA), small nuclear RNA (snRNA), messenger RNA (mRNA), ribosomal RNA (rRNA), long non-coding RNAs (lncRNA), and transfer RNA (tRNA).
   31. The method of paragraph 30, wherein the RNA comprises mRNA.
   32. The method of any one of the preceding paragraphs, wherein the target DNA molecule comprises a barcode.
   33. The method of any one of the preceding paragraphs, wherein the target DNA molecule comprises one or more of
(i) a unique molecular identifier (UMI);
(ii) a barcode;
(iii) a sequence of at least a portion of a target analyte, or a complement thereof; and
(iv) a capture sequence.
   34. The method of any one of the preceding paragraphs, further comprising determining the nucleic acid sequence of the displaced DNA molecule.
   35. The method of paragraph 34, wherein determining the nucleic acid sequence comprises determining the sequence of one or more of
(i) a barcode or a complement thereof;
(ii) all or a portion of a target analyte or a complement thereof; and/or
(iii) a molecular identifier (UMI).
   36. The method of paragraph 35, further comprising using the determined sequences of (i), (ii) and optionally (iii) to identify the abundance of the target nucleic acid analyte in a biological sample.
   37. The method of any one of the preceding paragraphs, wherein the size of the first primer or second strand of the double stranded adapter is between about 8 and about 100 nucleotides, inclusive.
   38. The method of any one of the preceding paragraphs, wherein the second hybrid region comprises between about four and about 30 RNA nucleotides, inclusive.
   39. The method of paragraph 38, wherein the first region comprises between about four and about 30 DNA nucleotides, inclusive.
   40. The method of paragraph 8, further comprising, after step (a), substantially removing unbound adapter from the target nucleic acid or first combined molecule, optionally wherein the removing comprises contacting the target nucleic acid or first combined molecule with a wash buffer.
   41. The method of any one of the preceding paragraphs, wherein the target DNA molecule comprises a region complementary to a poly(A) nucleotide sequence, or a complement thereof.
   42. The method of any one of the preceding paragraphs, wherein the target DNA molecule comprises a nucleotide sequence of a coding region of an mRNA, or a complement thereof.
   43. The method of any one of the preceding paragraphs, wherein the target DNA molecule comprises one or more functional domains.
   44. The method of paragraph 43, wherein the functional domain comprises an amplification domain or a primer-binding site.
   45. The method of any one of the preceding paragraphs, wherein the target DNA molecule comprises one or more priming sites for a sequencing primer.
   46. The method of any one of the preceding paragraphs, wherein the target DNA molecule is derived from a tissue sample.
   47. The method of paragraph 46, wherein the tissue sample is derived from a mammalian subject.
   48. The method of paragraph 47, wherein the subject is a human.
   49. The method of any one of the preceding paragraphs, wherein target DNA molecule is from a fixed sample.
   50. The method of paragraph 49, wherein the fixed sample comprises a Formalin-Fixed Paraffin-Embedded (FFPE) sample.
   51. The method of any one of the preceding paragraphs, wherein the method further comprises, prior to step (a) one or more steps of de-crosslinking the sample.
   52. The method of paragraph 51, wherein the sample comprises a FFPE sample, and wherein the method further comprises, prior to step (a), deparaffinizing the tissue sample.
   53. The method of any one of paragraphs 71-79, further comprising, prior to step (a), one or more steps of staining and/or labelling the biological sample.
   54. A primer set comprising:
(a) a first primer comprising:
   (i) a first region, comprising a dsDNA strand displacing DNA sequence that is substantially complementary to a first primer binding site of a target nucleic acid, and
   (ii) a second hybrid region comprising one or more of RNA nucleotides, DNA nucleotides comprising a unique target sequence, or inosine nucleotides; and
(b) a second primer comprising the second hybrid region of the first primer.
   55. The primer set of paragraph 54, wherein the second primer consists of the second hybrid region of the first primer.
   56. The primer set of paragraph 54 or 55, wherein the size of the first primer is between about 8 and about 100 nucleotides, inclusive.
   57. The primer set of any one of paragraphs 54 to 56, wherein the first primer comprises a first region comprising between about four and about 30 nucleotides, inclusive.
   58. The primer set of any one of paragraphs 54 to 57, wherein the first primer comprises a second region comprising between about four and about 30 nucleotides, inclusive.
   59. The primer set of any one of paragraphs 54-58, wherein the first region comprises one or more modified nucleic acids.
   60. The method of paragraph 59, wherein the first region comprises one or more locked nucleic acids (LNAs).
   61. The method of paragraph 59 or 60, wherein the first region comprises one or more super G (8-aza-7-deazaguanosine) nucleic acids.
   62. The primer set of any one of paragraphs 54-61, wherein the second region of the first primer comprises RNA nucleotides.
   63. The primer set of any one of paragraphs 54-62, wherein the second region of the first primer comprises inosine nucleotides.
   64. The primer set of any one of paragraphs 54-63, wherein the second region of the first primer comprises a unique target sequence, and wherein the target sequence comprises a nickase recognition sequence.
   65. The primer set of any one of paragraphs 54-64, wherein the second region of the first primer comprises a methylated GATC nucleotide sequence.
   66. A kit comprising
(a) a first primer comprising:
   (i) a first region, comprising a dsDNA strand displacing DNA sequence that is substantially complementary to a first primer binding site of a target nucleic acid, and
   (ii) a second hybrid region comprising one or more of RNA nucleotides, DNA nucleotides comprising a unique target sequence, or inosine nucleotides; and
(b) instructions for performing the method of any one of paragraphs 1-53.
   67. The kit of paragraph 66, wherein the first region comprises one or more modified nucleic acids.
   68. The kit of paragraph 66 or 67, wherein the first region comprises one or more locked nucleic acids (LNAs).
   69. The kit of any one of paragraphs 66-68, wherein the first region comprises one or more super G (8-aza-7-deazaguanosine) nucleic acids.
   70. The kit of any one of paragraphs 66-69, wherein the second region of the first primer comprises RNA nucleotides.
   71. The kit any one of paragraphs 66-70, further comprising a second primer comprising the second region of the first primer.
   72. The kit of any one of paragraphs 66-71, further comprising an RNA-specific nuclease enzyme.
   73. The kit of paragraph 72, wherein the RNA-specific nuclease enzyme comprises RNaseH.
   74. The kit of paragraph 66, wherein the second region of the first primer comprises inosine nucleotides.
   75. The kit of paragraph 74, further comprising an endonuclease enzyme that degrades the inosine nucleotides.
   76. The kit of paragraph 75, wherein the enzyme comprises Endonuclease V.
   77. The kit of paragraph 66, wherein the second region of the first primer comprises one or more unique target sequences.
   78. The kit of paragraph 77, further comprising a nickase enzyme that selectively cleaves or removes the unique target sequences.
   79. The kit of paragraph 66, wherein the second region of the first primer comprises one or more methylated GATC nucleotide sequences.
   80. The kit of paragraph 79, further comprising a methylation-specific nuclease enzyme.
   81. The kit of paragraph 80, wherein the methylation-specific nuclease enzyme comprises DpnI.
   82. The kit of any one of paragraphs 66-81, further comprising a single stranded adapter or a double stranded adapter.
   83. The kit of paragraph 82, wherein the adapter comprises bases complementary to the second region of the first primer.
   84. The kit of paragraph 83, wherein the first adapter comprises a 5′phosphate.
   85. The kit of paragraph 84, further comprising a ligase enzyme.
   86. The kit of paragraph 85, wherein the ligase enzyme comprises a T4 ligase.
   87. The kit of any one of paragraphs 66-86, further comprising a strand displacing DNA polymerase.
   88. The kit of paragraph 87, wherein the strand-displacing polymerase is selected from the group consisting of Bst2.0, Bst3.0, Bsu and Phil29.
   89. The kit of any one of paragraphs 66-88, further comprising one or more of
(a) a solvent suitable to solubilize unbound first primer(s) and/or unbound second primer(s);
(b) one or more DNA-specific Polymerase enzymes;
(c) one or more nuclease enzymes;
(d) buffers and/or reagents suitable for enzyme reactions according to the methods of any one of paragraphs 1-55.
   90. A partially double stranded nucleic acid adapter, comprising:
(I) a first adapter strand, comprising:
   (i) a first region, comprising DNA nucleotides; and
   (ii) a second hybrid 5′ overhang region, comprising RNA nucleotides, or DNA nucleotides comprising a unique target sequence, or inosine nucleotides;
      and
(II) a second adapter strand comprising a 3′ overhang DNA splint sequence, wherein the splint sequence is complementary to all or part of the first adapter strand and complementary to 5′ end of a target dsDNA molecule.
   91. The adapter of paragraph 90, wherein the first adapter strand comprises between about 8 and about 100 nucleotides, inclusive.
   92. The adapter of paragraph 90 or 91, wherein the first region of the first adapter strand comprises between about four and about 30 nucleotides, inclusive.
   93. The adapter of any one of paragraphs 90 to 92, wherein the first region of the second adapter strand comprises between about four and about 30 nucleotides, inclusive.
   94. The adapter of any one of paragraphs 90 to 93, wherein the second hybrid 5′ overhang region comprises between about four and about 30 nucleotides, inclusive.
   96. The adapter of any one of paragraphs 90 to 94, wherein the second hybrid 5′ overhang region comprises RNA nucleotides.
   97. The adapter of any one of paragraphs 90 to 94, wherein the second hybrid 5′ overhang region comprises inosine nucleotides.
   98. The adapter of any one of paragraphs 90 to 94, wherein the second hybrid 5′ overhang region comprises a unique target sequence, and wherein the target sequence comprises a nickase recognition sequence.
   99. The adapter of any one of paragraphs 90 to 94, wherein the second hybrid region comprises a methylated GATC nucleotide sequence.
   100. A method for amplification of a target DNA molecule from a template-switching workflow, comprising:
(a) forming a target ssDNA molecule from a template-switching workflow, comprising attaching a template switching oligonucleotide to a captured probe to form the target nucleic acid,
   wherein the template switching oligonucleotide further comprises a second primer binding sequence, and
   wherein the generated target ssDNA molecule further comprises the second primer binding sequence or a complement thereof;
(b) hybridizing a second primer to the second primer binding sequence of the target ssDNA molecule to form a hybridized second molecule comprising a hybridized second primer,
   wherein the second primer comprises a second hybrid region comprising one or more of RNA nucleotides, DNA nucleotides comprising a unique target sequence, or inosine nucleotides; and
(c) extending the hybridized second primer of the hybridized second molecule using a strand-displacing DNA polymerase to provide an extended hybrid double-stranded (ds) DNA molecule;
(d) removing the second primer from the extended hybrid double-stranded (ds) DNA molecule to form a truncated dsDNA molecule;
(e) hybridizing a second primer to the second primer binding sequence of the truncated dsDNA molecule to form a hybridized second molecule comprising a hybridized second primer; and
(f) extending the hybridized second primer of the hybridized second molecule using a strand-displacing DNA polymerase to provide an extended hybrid double-stranded (ds) DNA molecule and a displaced single-stranded (ss) DNA molecule.
   101. A method for amplification of a target DNA molecule, comprising:
(a) forming a combined first molecule from a target double-stranded (ds) DNA molecule and a nucleic acid adapter,
   wherein the target dsDNA molecule comprises a first DNA strand and a second DNA strand, the forming comprising:
   (i) linking to a 3′ end of the second DNA strand of the target dsDNA molecule a 5′ end of a second strand of the nucleic acid adapter, to form a linked second adapter strand, and
   (ii) hybridizing a first strand of the nucleic acid adapter to the linked second adapter strand to form the combined first molecule,
      wherein the first strand of the nucleic acid adapter comprises
      (I) a first region comprising DNA nucleotides; and
      (II) a second hybrid region, comprising RNA nucleotides, DNA nucleotides comprising a unique target nucleotide sequence, or inosine nucleotides, or a methylated GATC nucleotide sequence,
      wherein the first strand of the nucleic acid adapter comprises a nucleotide sequence complementary to all or part of the second strand of the nucleic acid adapter;
      optionally wherein a 5′ end of the second strand of the nucleic acid adapter is linked to a 3′ end of the second DNA strand of the dsDNA molecule via a phosphodiester linkage;
(b) removing the second hybrid region from the combined first molecule to form a truncated DNA molecule comprising a region of ssDNA;
(c) hybridizing a second primer to the region of ssDNA of the truncated DNA molecule to form a hybridized second molecule comprising a hybridized second primer,
   wherein the second primer comprises the second hybrid region comprising RNA nucleotides, DNA nucleotides comprising a unique target sequence, or inosine nucleotides;
(d) extending the hybridized second primer with a strand-displacing DNA polymerase to provide an extended hybrid dsDNA molecule and a displaced DNA molecule.
   102. A method for amplification of a target DNA molecule, the method comprising:
(a) extending a capture probe using a target nucleic acid analyte hybridized thereto as a template, thereby generating an extended capture probe comprising all or a portion of a complement of the target nucleic acid analyte, optionally wherein the target nucleic acid analyte is RNA;
(b) performing a template switching reaction using (i) the extended capture probe and (ii) a template switching oligonucleotide comprising a first primer binding sequence at its 5′ end, thereby generating a target DNA molecule comprising the extended capture probe and a complement of the first primer binding sequence;
(c) hybridizing a second primer to the complement of the first primer binding sequence of the target DNA molecule, wherein the second primer comprises a second hybrid region corresponding to the first primer binding sequence, and wherein the second hybrid region comprises RNA nucleotides, or DNA nucleotides comprising a unique target sequence, or inosine nucleotides, or a methylated GATC nucleotide sequence;
(d) extending the second primer using the target DNA molecule as a template, to form a double stranded (ds) hybrid DNA molecule;
(e) removing the second hybrid region of the second primer from the ds hybrid DNA molecule to form a truncated DNA molecule comprising a ssDNA region;
(f) hybridizing a second primer comprising the second hybrid region to the ssDNA region of the truncated DNA molecule to form a hybridized second primer; and
(g) extending the hybridized second primer with a strand-displacing DNA polymerase to provide an extended nucleic acid molecule and a displaced DNA molecule.
   103. A partially double stranded nucleic acid adapter molecule, comprising:
(i) a first adapter strand, consisting of a first region of DNA nucleotides; and
(ii) a second adapter strand, comprising a DNA sequence complementary to the first region of DNA nucleotides of the first strand and a second region of DNA nucleotides,
   wherein the second region comprises at least 5 nucleotides;
   wherein the second adapter strand is hybridized to the first region of DNA nucleotides of the first adapter strand to form the partially ds nucleic acid adapter comprising a 3′ overhang, and
   wherein 3′ overhang comprises the second region of DNA nucleotides of the first adapter strand,
optionally wherein the second adapter strand is linked to the 3′ end of the second DNA strand via a 5′ phosphodiester linkage.

The entire contents of all references (including literature references, issued patents, published patent applications, and co pending patent applications) cited throughout this application are hereby expressly incorporated by reference.

Unless defined otherwise, all technical and scientific terms used herein have the same meanings as commonly understood by one of skill in the art to which the disclosed subject matter belongs. Publications cited herein and the materials for which they are cited are specifically incorporated by reference.

Those skilled in the art will recognize or be able to ascertain using no more than routine experimentation, many equivalents to the specific embodiments of the subject matter described herein.

We claim:

1. A method for amplification of a target DNA molecule, comprising:
(a) hybridizing a first primer to a first primer binding site in a target DNA molecule, the first primer comprising:
(i) a first region, comprising a DNA sequence that is substantially complementary to the first primer binding site of the target DNA molecule; and
(ii) a second hybrid region comprising one or more of RNA nucleotides, DNA nucleotides comprising a unique target sequence, or inosine nucleotides,
wherein the target DNA molecule comprises single stranded (ss) DNA comprising the first primer binding site;
(b) extending the first primer from its 3′ end using the target DNA molecule as a template and extending 3′ end of the target DNA molecule using the first primer as a template, to form an extended hybrid double-stranded (ds) DNA molecule;
(c) removing the second hybrid region from the extended hybrid dsDNA molecule to form a truncated dsDNA molecule comprising a region of ssDNA;
(d) hybridizing a second primer to the region of ssDNA of the truncated dsDNA molecule to form a hybridized second molecule comprising a hybridized second primer,
wherein the second primer comprises the second hybrid region comprising one or more of RNA nucleotides, DNA nucleotides comprising a unique target sequence, or inosine nucleotides; and
(e) extending the hybridized second primer of the hybridized second molecule using a strand-displacing DNA polymerase to provide an extended hybrid double-stranded (ds) DNA molecule and a displaced single-stranded (ss) DNA molecule.

2. The method of claim 1, wherein the first region is located at the 3′ end of the first primer, and/or wherein the first primer binding site is located at the 3′ end of the target DNA molecule.

3. The method of claim 1, wherein the second hybrid region of the first primer forms a single stranded 5′ overhang when the first primer hybridizes to the target DNA molecule.

4. The method of claim 1, wherein the first primer comprises in 5′ to 3′ orientation: the second hybrid region and the first region.

5. The method of claim 1, wherein a 5′ end of the target DNA molecule is conjugated to a substrate.

6. A method for amplification of a target DNA molecule, comprising:
(a) forming a combined first molecule from a target double stranded (ds) DNA molecule comprising a first DNA strand and a second DNA strand, the forming comprising:
(i) linking to a 3′ end of the second DNA strand of the target dsDNA molecule a nucleic acid adapter, the nucleic acid adapter comprising:
(I) a first adapter strand, comprising:
(i) a first region, comprising DNA nucleotides; and
(ii) a second hybrid region, comprising RNA nucleotides, or DNA nucleotides comprising a unique target sequence, or inosine nucleotides;
and
(II) a second adapter strand comprising a DNA sequence complementary to all or part of the first strand;
wherein the first adapter strand is hybridized to the second adapter strand;
optionally wherein the second adapter strand is linked to the 3′ end of the second DNA strand via a 5′ phosphodiester linkage;
(b) removing the second hybrid region from the combined first molecule to form a truncated DNA molecule comprising a region of ssDNA;
(c) hybridizing a second primer to the region of ssDNA of the truncated DNA molecule to form a hybridized second molecule comprising a hybridized second primer,
wherein the second primer comprises the second hybrid region comprising RNA nucleotides, DNA nucleotides comprising a unique target sequence, or inosine nucleotides;
(d) extending the hybridized second primer with a strand-displacing DNA polymerase to provide an extended hybrid dsDNA molecule and a displaced DNA molecule.

7. A method for amplification of a target DNA molecule, comprising:
(a) forming a combined first molecule having a 3′overhang from a target double stranded (ds) DNA molecule having a first DNA strand and a second DNA strand, the forming comprising: linking to a 5′ end of the first DNA strand of the target dsDNA molecule a 3′ end of a first adapter strand of a partially ds nucleic acid adapter, the partially ds nucleic acid adapter comprising:
(i) a first adapter strand, consisting of a first region of DNA nucleotides; and
(ii) a second adapter strand, comprising a DNA sequence complementary to the first region of DNA nucleotides of the first strand and a second region of DNA nucleotides,
wherein the second region comprises at least 5 nucleotides;
wherein the second adapter strand is hybridized to the first region of DNA nucleotides of the first adapter strand to form the partially ds nucleic acid adapter comprising a 3′ overhang, and
wherein 3′ overhang comprises the second region of DNA nucleotides of the first adapter strand;
optionally wherein the second adapter strand is linked to the 3′ end of the second DNA strand via a 5′ phosphodiester linkage;
(b) hybridizing a second primer to 3′ overhang of the combined first molecule to form a hybridized second molecule comprising a hybridized second primer,
wherein the second primer comprises a sequence complementary to the second region of DNA nucleotides of the first adapter strand, and
wherein the second primer comprises RNA nucleotides, DNA nucleotides comprising a unique target sequence, or inosine nucleotides;
(c) extending the hybridized second primer with a strand-displacing DNA polymerase to provide an extended hybrid dsDNA molecule and a displaced ssDNA molecule.

8. The method claim 7, wherein forming in step (a) comprises blunt end ligating the second adapter strand to the second DNA strand of the target dsDNA molecule.

9. The method of claim 7, wherein the linking to a 3′ end of the first DNA strand of the dsDNA molecule a nucleic acid adapter comprises ligation,
optionally wherein the ligation comprises enzymatic ligation.

10. The method of claim 1, wherein the second hybrid region comprises RNA nucleotides, and
wherein removing the second hybrid region comprises contacting the second hybrid region with an RNase enzyme, optionally RNase H.

11. The method of claim 1, wherein the second hybrid region comprises one or more inosine nucleotides, and
wherein removing the second hybrid region comprises contacting the second hybrid region with an endonuclease enzyme that cleaves the one or more inosine nucleotides, optionally wherein the endonuclease comprises Endonuclease V.

12. The method of claim 1, wherein the second hybrid region comprises one or more unique target sequences,
wherein the one or more unique target sequences are not present within the target DNA molecule, and
wherein removing the second hybrid region comprises contacting the second hybrid region with a nickase enzyme that selectively cleaves or removes the one or more unique target sequences.

13. The method of claim 1, wherein the second hybrid region comprises one or more methylated GATC sequences, and wherein removing the second hybrid region comprises contacting the second region with a methylation-specific nuclease, optionally wherein the methylation-specific nuclease comprises DpnI.

14. The method of claim 1, wherein forming in step (a) comprises contacting the target DNA molecule with a reaction mixture comprising the first primer, or partially double stranded adapter, or double stranded adapter and the strand displacing DNA polymerase, optionally wherein the reaction mixture further comprises one or more of:
(i) an enzyme specific for the second hybrid region;
(ii) and the second primer;
(iii) the second primer;
(iv) the enzyme specific for the second hybrid region; and
(v) the strand displacing DNA polymerase.

15. The method of claim 1, further comprising repeating each of the steps of removing, hybridizing and extending the second primer with a strand-displacing DNA polymerase once or more than once to provide a multiplicity of copies of the target DNA molecule,
optionally wherein each of the steps of removing, hybridizing and extending the second primer with a strand-displacing DNA polymerase is carried out at a constant temperature.

16. The method of claim 1, wherein the target DNA molecule comprises a spatial barcode and/or a DNA sequence of at least a portion of a target nucleic acid analyte from a sample, or a complement thereof, wherein the target nucleic acid analyte comprises a nucleic acid,
optionally wherein the analyte is selected from genomic DNA, RNA, synthetic DNA, or synthetic RNA.

17. The method of claim 1, wherein the target DNA molecule is directly or indirectly conjugated to a substrate or matrix,
optionally wherein the substrate or matrix comprises a solid support or a gel, and
wherein the substrate comprises an array comprising a multiplicity of capture probes.

18. The method of claim 1, wherein the target DNA molecule comprises a spatial barcode and/or at least a portion of a target nucleic acid analyte or a complement thereof, and wherein
(i) a substrate is conjugated to 5′end of the target DNA molecule; and
(ii) the first primer binding site is located at the 3′ end of the target DNA molecule.

19. The method of claim 7, wherein the target DNA molecule comprises a spatial barcode and/or at least a portion of a target nucleic acid analyte or a complement thereof, and
wherein
(i) a substrate is conjugated to 5′end of a first strand of the target DNA molecule; and
(ii) the second strand of the double stranded nucleic acid adapter is linked to 3′ end of the first strand of the target dsDNA molecule.

20. The method of claim 1, further comprising determining the nucleic acid sequence of the displaced DNA molecule, optionally wherein determining the nucleic acid sequence comprises determining the sequence of one or more of
(i.) a spatial barcode or a complement thereof;
(ii.) all or a portion of a target analyte or a complement thereof; and/or
(iii.) a molecular identifier (UMI).
